(19) United States
(12) Patent Application Publication
(10) Pub. No.: US 2025/0388915 A1
(43) Pub. Date: Dec. 25, 2025

(54) METHODS AND COMPOSITIONS FOR INTRON MEDIATED- EXPRESSION OF REGULATORY ELEMENTS FOR TRAIT DEVELOPMENT

(71) Applicant: InEdita Bio, Inc., Dover, DE (US)

(72) Inventors: Viviane Cristina HEINZEN DE SILVA, Florianópolis, SC (BR); Paulo ARRUDA, Campinas, São Paulo (BR)

(21) Appl. No.: 18/844,880

(22) PCT Filed: Mar. 6, 2023
(86) PCT No.: PCT/US2023/063791

§ 371 Date: Sep. 6, 2024

Related U.S. Application Data
(60) Provisional application No. 63/317,425, filed on Mar. 7, 2022.
(60) Provisional application No. 63/377,701, filed on Sep. 29, 2022.

(51) Int. Cl. C12N 15/82; C12N 9/22; C12N 15/11

(52) U.S. Cl. CPC C12N 15/8213; C12N 9/226; C12N 15/11; C12N 15/8205; C12N 15/8218; C12N 2310/14; C12N 2310/20

(57) ABSTRACT

Disclosed are compositions and methods for a non-coding nucleic acid gene editing platform for the delivery of regulatory nucleic acid sequences and small peptides in a cell. In a particular aspect, provided herein is a non-coding nucleic acid gene editing platform to down regulate endogenous genes and genes from pests and pathogens causing diseases. In another aspect, the non-coding nucleic acid gene editing platform described herein is useful to deliver small regulatory peptides encoded from nucleic acid sequences embedded in a non-coding nucleic acid of a gene. More specifically, the non-coding nucleic acid gene editing platform provided herein allows using non-coding nucleic acid from any gene to deliver regulatory nucleic acids and small peptides in a cell. In another aspect, such regulatory nucleic acids and small peptides are useful to develop traits to enhance crop quality and yield.

### CROSS-REFERENCE TO RELATED APPLICATION

This application is a U.S. National Application of International Application No. PCT/US2023/063791, filed Mar. 6, 2023, which claims priority to U.S. provisional application, 63/317,425 filed Mar. 7, 2022, and U.S. provisional application, 63/377,701 filed Sep. 29, 2022, the entirety of which is incorporated by reference herein.

### SEQUENCE LISTING

The instant application contains a Sequence Listing which has been submitted electronically in XML format and is hereby incorporated by reference in its entirety. Said XML copy, created on Apr. 22, 2025, is named 65864-701_831_SL.xml and is 3,412,529 bytes in size.

### BACKGROUND

Plants with high crop quality and yield are desired by both farmers and consumers. As the global population continues to grow, food must be produced in a sustainable and increased manner in order to satisfy the demand of the growing population. Therefore, there is a need for the development of genetically edited plants with improved biotechnological traits, such as enhanced crop quality, yield, pest resistance, disease resistance, chemical resistance, photosynthetic efficiency, and the like.

### SUMMARY

In various aspects, provided herein are plants harboring such improved biotechnological traits. For instance, plants having cells with modified non-coding regions such as introns comprising an endogenous or exogenous nucleic acid that, when expressed, confers one or more desired traits to the plant. In certain instances, the nucleic acid is exogenous to the non-coding region, such as an intron. In certain instances, the modified non-coding regions are genetically edited. As a non-limiting example, the non-coding region is or has been genetically edited using a CRISPR-Cas based method. As such, modified non-coding regions include non-coding regions that are or have been genetically edited.

In one aspect, provided herein is a system comprising a first nucleic acid sequence comprising a nucleic acid encoding a ribonucleic acid or a peptide, a second nucleic acid sequence comprising a sequence encoding a DNA nuclease, and a third nucleic acid sequence comprising a sequence encoding a guide RNA, wherein the guide RNA is complementary to a non-coding region of the genome of a cell. In some embodiments, the nucleic acid encodes the ribonucleic acid, and the ribonucleic acid specifically binds to (i) a target nucleic acid of Table 6, (ii) a target nucleic acid present in a pest of Table 6, (iii) a target nucleic acid of an organism of Table 6, (iv) a target nucleic acid exogenous or endogenous to the cell, (v) a target nucleic acid responsible for water acquisition, nutrient acquisition, disease control, or pest control, or any combination of two or more thereof, in the cell, (v) a target nucleic acid comprises a regulatory element involved in: plant growth and development, yield, biotic stress, abiotic stress, or herbicide resistance, or any combination of two or more thereof, (vi) a target nucleic acid of an insect, bacteria, fungi, or worm, or a combination of two or more thereof, that is harmful to the cell, (vii) a target nucleic acid of an organism that causes a disease to the cell, or (viii) a combination of two or more of (i) to (vii). In some embodiments, the nucleic acid encodes the ribonucleic acid, and the nucleic acid comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of any one of the target gene sequences of Table 6; and/or comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to at least 10 contiguous bases of any one of the target gene sequences of Table 6. In some embodiments, the nucleic acid encodes the peptide, and the peptide is (i) a peptide selected from Table 7, (ii) a peptide encoded by an mRNA sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8, (iii) a peptide that affects hormonal regulation, protection against a pathogen, protection against an insect, nitrogen fixation, nutrient acquisition, immunity induction, biotic stress, or abiotic stress, or a combination of two or more thereof, in the cell, or (iv) a combination of two or more of (i) to (iii). In some embodiments, the nucleic acid encodes the peptide, and the nucleic acid comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8. In some embodiments, the non-coding region is positioned within, or adjacent to, a gene of the cell. In some embodiments, the gene is actin, ubiquitin, ribosomal gene, gene encoding a heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, or a gene selected from Table 1. In some embodiments, the non-coding region is selected from Table 2. In some embodiments, the non-coding region comprises a site recognized by the DNA nuclease (nuclease recognition site). In some embodiments, the nuclease recognition site comprises a protospacer adjacent motif (PAM). In some embodiments, the nuclease recognition site is selected from Table 3. In some embodiments, the gRNA is complementary to about 17 to about 22 nucleotides of the non-coding region. In some embodiments, the gRNA comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 4. In some embodiments, the system comprises a plasmid, wherein the second nucleic acid and the third nucleic acid are present in the plasmid. In some embodiments, (i) the first nucleic acid comprises a first nuclease cleavage site and a second nucleic cleavage site, and the nucleic acid encoding the ribonucleic acid or the peptide is positioned between the first nuclease cleavage site and the second nuclease cleavage site, optionally wherein the first nuclease cleavage site and the second nuclease cleavage site are recognized by the DNA nuclease; (ii) the first nucleic acid is a blunt linear double-stranded oligodeoxynucleotide (dsODN) encoding the ribonucleic acid or the peptide; (iii) the first nucleic acid is a chemically modified dsODN encoding the ribonucleic acid or a peptide, optionally comprising a phosphorothioate linkage and/or 5′ phosphorylation; or (iv) the first nucleic acid is a blunt single-stranded oligodeoxynucleotide (ssODN) encoding the ribonucleic acid or the peptide. In some embodiments, the DNA nuclease is a CRISPR-Cas nuclease.

In one aspect, provided herein is a method of inserting the nucleic acid encoding the ribonucleic acid or the peptide into the non-coding region of the cell, the method comprising introducing the system as described herein into the cell. In some embodiments, the cell comprises the nucleic acid encoding the ribonucleic acid or the peptide as described herein positioned within the non-coding region of the genome of the cell. In some embodiments, the non-coding region is adjacent to a gene encoding a mRNA, and after transcription of the gene and mRNA splicing, the mRNA is translated into a protein endogenous to the cell.

In one aspect, provided herein is a cell comprising a recombinant nucleic acid comprising a coding region and a non-coding region, wherein the non-coding region comprises a nucleic acid exogenous to the non-coding region, and wherein the coding region is the coding region of a gene, and the gene (i) is actin, ubiquitin, ribosomal gene, gene encoding a heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, or a gene selected from Table 1; (ii) accounts for about 1% to about 20% of gene expression in the cell; (iii) is transcribed from a constitutive promoter, optionally wherein the promoter is specific or a plant organ or tissue, further optionally wherein the organ or tissue comprises a root, stem, fruit, seed, leaf, ground tissue, vascular tissue, or dermal tissue, or a combination of two or more thereof; or (iv) a combination of two or more of (i) to (iii). In some embodiments, the non-coding region comprises (i) an intron positioned between a first exon region of the coding region and a second exon region of the coding region, (ii) a 5′ non-coding region positioned adjacent to the coding region, or (iii) a 3′ non-coding region positioned adjacent to the coding region. In some embodiments, the gene encodes mRNA endogenous to the cell, and after transcription of the gene and mRNA splicing, the mRNA is translated into a protein endogenous to the cell. In some embodiments, the gene is constitutively expressed in the cell. In some embodiments, the nucleic acid exogenous to the non-coding region encodes a ribonucleic acid or a peptide. In some embodiments, the nucleic acid encodes the ribonucleic acid, and the ribonucleic acid specifically binds to (i) a target nucleic acid of Table 6, (ii) a target nucleic acid present in a pest of Table 6, (iii) a target nucleic acid of an organism of Table 6, (iv) a target nucleic acid exogenous or endogenous to the cell, (v) a target nucleic acid responsible for water acquisition, nutrient acquisition, disease control, or pest control, or any combination of two or more thereof, in the cell, (vi) a target nucleic acid comprises a regulatory element involved in: plant growth and development, yield, biotic stress, abiotic stress, or herbicide resistance, or any combination of two or more thereof, (vii) a target nucleic acid of an insect, bacteria, fungi, or worm, or a combination of two or more thereof, that is harmful to the cell, (viii) a target nucleic acid of an organism that causes a disease to the cell, or (ix) a combination of two or more of (i) to (viii). In some embodiments, the nucleic acid encodes the ribonucleic acid, and the nucleic acid comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of any one of the target gene sequences of Table 6; and/or comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to at least 10 contiguous bases of any one of the target gene sequences of Table 6. In some embodiments, the nucleic acid encodes the peptide, and the peptide is (i) a peptide selected from Table 7, (ii) a peptide encoded by an mRNA sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8, (iii) a peptide that affects hormonal regulation, protection against a pathogen, protection against an insect, nitrogen fixation, nutrient acquisition, immunity induction, biotic stress, or abiotic stress, or a combination of two or more thereof, in the cell, or (iv) a combination of two or more of (i) to (iii). In some embodiments, the nucleic acid encodes the peptide, and the nucleic acid comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8. In some embodiments, the non-coding region comprises a nuclease recognition site, optionally wherein the nucleic recognition site comprises a protospacer adjacent motif (PAM). In some embodiments, the nucleic acid exogenous to the non-coding region is endogenous or exogenous to the cell. In some embodiments, the genome of the cell comprises the recombinant nucleic acid. In some embodiments, the nucleic acid exogenous to the non-coding region is about 10 to about 700 bases in length, or about less than 200 bases in length.

In one aspect, provided herein is a cell comprising a recombinant nucleic acid comprising a coding region and a non-coding region, wherein the non-coding region comprises a nucleic acid exogenous to the non-coding region, and wherein the nucleic acid exogenous to the non-coding region encodes a ribonucleic acid that specifically binds to (i) a target nucleic acid of Table 6, (ii) a target nucleic acid present in pest of Table 6, (iii) a target nucleic acid of an organism of Table 6, (iv) a target nucleic acid exogenous or endogenous to the cell, (v) a target nucleic acid responsible for water acquisition, nutrient acquisition, disease control, or pest control, or any combination of two or more thereof, in the cell, (vi) a target nucleic acid comprises a regulatory element involved in: plant growth and development, yield, biotic stress, abiotic stress, or herbicide resistance, or any combination of two or more thereof, (vii) a target nucleic acid of an insect, bacteria, fungi, or worm (e.g., larva of the insect, and nematode), or a combination of two or more thereof, that is harmful to the cell, (viii) a target nucleic acid of an organism that causes a disease to the cell, or (ix) a combination of two or more of (i) to (viii).

In one aspect, provided herein is a cell comprising a recombinant nucleic acid comprising a coding region and a non-coding region, wherein the non-coding region comprises a nucleic acid exogenous to the non-coding region, and wherein the nucleic acid exogenous to the non-coding region comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of any one of the target gene sequences of Table 6; of comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to at least 10 contiguous bases of any one of the target gene sequences of Table 6.

In one aspect, provided herein is a cell comprising a recombinant nucleic acid comprising a coding region and a non-coding region, wherein the non-coding region comprises a nucleic acid exogenous to the non-coding region, and wherein the nucleic acid exogenous to the non-coding region encodes a peptide, and the peptide is (i) a peptide selected from Table 7, (ii) a peptide encoded by an mRNA sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8, (iii) a peptide that affects hormonal regulation, protection against a pathogen, protection against an insect, nitrogen fixation, nutrient acquisition, immunity induction, biotic stress, or abiotic stress, or a combination of two or more thereof, in the cell, or (iv) a combination of two or more of (i) to (iii).

In one aspect, provided herein is a cell comprising a recombinant nucleic acid comprising a coding region and a non-coding region, wherein the non-coding region comprises a nucleic acid exogenous to the non-coding region, and wherein the nucleic acid exogenous to the non-coding region comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8. In some embodiments, the non-coding region is positioned within, or adjacent to, a gene of the cell. In some embodiments, the gene is actin, ubiquitin, ribosomal gene, gene encoding a heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, or a gene selected from Table 1. In some embodiments, the non-coding region is selected from Table 2. In some embodiments, the recombinant nucleic acid is positioned within the genome of the cell. In some embodiments, the cell is a plant cell, and optionally the plant is a plant of Table 9, and further optionally the plant cell is a ground tissue cell, a vascular tissue cell, or a dermal tissue cell. In some embodiments, the cell is not transgenic.

In one aspect, provided herein is a plant comprising the cell as described herein, optionally wherein the plant is a plant of Table 9. In some embodiments, the plant is resistant or more resistant to a pest, disease, or chemical, or a combination of two or more thereof, as compared to a plant that does comprise the cell with the recombinant nucleic acid. In some embodiments, the plant has an improved nutritional quality, increased crop yield, more efficient nutrient acquisition, or more efficient photosynthetic efficiency, or a combination of two or more thereof, as compared to a plant that does not comprise the cell with the recombinant nucleic acid.

In one aspect, provided herein is a seed of the plant as described herein.

In one aspect, provided herein is a method of reducing or eliminating expression of a target gene in the cell as described herein, the method comprising introducing into the non-coding region of the cell the nucleic acid exogenous to the non-coding region, wherein nucleic acid exogenous to the non-coding region encodes for a sequence that binds to mRNA of the target gene, thereby reducing or eliminating expression of the target gene.

In one aspect, provided herein is a method of regulating a target gene or peptide in the cell as described herein, the method comprising introducing into the non-coding region of the cell the nucleic acid exogenous to the non-coding region, wherein the nucleic acid exogenous to the non-coding region encodes for an amino acid sequence that is capable of regulating the target gene or peptide in the cell, thereby regulating the target gene or peptide in the cell.

In one aspect, provided herein is a method of introducing, increasing, or reducing a trait in the plant as described herein, the method comprising introducing into the non-coding region of the cell of the plant the nucleic acid exogenous to the non-coding region, wherein the nucleic acid exogenous to the non-coding region encodes for a sequence that binds to mRNA of a target gene, thereby introducing, increasing, or reducing the trait in the plant.

In one aspect, provided herein is a method of introducing, increasing, or reducing a trait in the plant as described herein, the method comprising introducing into the non-coding region of the cell of the plant the nucleic acid exogenous to the non-coding region, wherein the nucleic acid exogenous to the non-coding region encodes an amino acid sequence that regulates a target gene or peptide in the cell, thereby introducing, increasing or reducing the trait in the plant.

In one aspect, provided herein is a cell comprising a non-coding region, wherein the non-coding region comprises an endogenous or exogenous nucleic acid, optionally, wherein the non-coding region comprises (i) a modified (e.g., genetically edited) intron region positioned between a first exon region and a second exon region, (ii) a 5′ non-coding region, or (iii) a 3′ non-coding region, or (iv) at least two of (i)-(iii). In some embodiments, the nucleic acid is exogenous to the non-coding region and endogenous to the cell. In some embodiments, the nucleic acid is exogenous to the non-coding region and exogenous to the cell. In some embodiments, the non-coding region comprises the modified (e.g., genetically edited) intron region positioned between the first exon region and the second exon region, and wherein the first exon region and the second exon region are regions of a gene. In some embodiments, the modified (e.g., genetically edited) non-coding region comprises the 5′ non-coding region, and the 5′ non-coding region is upstream of a gene. In some embodiments, the modified (e.g., genetically edited) non-coding region comprises the 3′ non-coding region, and the 3′ non-coding region is downstream of a gene. In some embodiments, the modified (e.g., genetically edited) non-coding region is modified from an intron of a gene. In some embodiments, the gene is endogenous or exogenous to the cell. In some embodiments, the endogenous or exogenous nucleic acid is positioned within the non-coding region of the gene, or within a portion of the non-coding region of the gene. In some embodiments, the endogenous or exogenous nucleic acid does not replace any nucleobases of the non-coding region of the gene. In some embodiments, the endogenous or exogenous nucleic acid replaces 1-10, 1-20, 10-30, or 10-40 nucleobases of the non-coding region of the gene. In some embodiments, the first modified (e.g., genetically edited) intron region comprises a first portion of the intron of the gene, the endogenous or exogenous nucleic acid, and a second portion of the intron of the gene. In some embodiments, the intron of the gene is selected from Table 2. In some embodiments, the gene is selected from the examples shown in Table 1. In some embodiments, the gene comprises a plurality of introns. In some embodiments, the plurality of introns is about 2, 3, 4, 5, 6, 7, 8, 9, 10, 11, 12, 13, 14, 15, 16, 17, 18, 19, 20 introns (e.g., as exemplified by genes from Table 1). In some embodiments, the non-coding region is present in the first, second, third, fourth, fifth, sixth, seventh, eighth, nineth, tenth, eleventh, twelfth, thirteenth, fourteenth, fifteenth, sixteenth, seventeenth, eighteenth, nineteenth, or twentieth intron of the gene, as applicable. In some embodiments, the first exon region and the second exon region are regions of the gene. In some embodiments, the non-coding region comprises the modified (e.g., genetically edited) intron region positioned between the first exon region and the second exon region, and wherein the first exon region and the second exon region are regions of a gene. In some embodiments, the non-coding region comprises the 5′ non-coding region, and the 5′ non-coding region is upstream of a gene. In some embodiments, the non-coding region comprises the 3′ non-coding region, and the 3′ non-coding region is downstream of a gene. In some embodiments, the first exon region and the second exon region are regions of a gene. In some embodiments, the gene is endogenous or exogenous to the cell. In some embodiments, the gene is constitutively expressed. In some embodiments, the gene is expressed in a specific tissue or organ.

In some embodiments, the cell is a plant cell, and the tissue or organ comprises a root, stem, fruit, seed, leaf, ground tissue, vascular tissue, or dermal tissue, or a combination of two or more thereof. In some embodiments, the gene is expressed at a range of 1-5%, 1-10%, 5-15%, or 5-20% of the total expressed genes in the cell (e.g., as determined by mRNA expression profiling of the said cell). In some embodiments, upon transcription and mRNA splicing, the native mRNA of the gene is translated into the native protein of the gene. In some embodiments, the gene encodes a native protein. In some embodiments, the native protein is actin, ubiquitin, ribosomal protein, heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, RB7, or any other protein expressed from a gene of Table 1. In some embodiments, the gene is selected from Table 1.

In some embodiments, the endogenous or exogenous nucleic acid is transcribed from a promoter. In some embodiments, the promoter is a promoter native to the gene. In some embodiments, the endogenous or exogenous nucleic acid is transcribed from a promoter. In some embodiments, the promoter is a constitutive promoter. In some embodiments, the promoter is specific for a plant organ. In some embodiments, the plant organ is a root, stem, fruit, seed, or leaf. In some embodiments, the promoter is specific to a plant tissue. In some embodiments, the plant tissue is a ground tissue, vascular tissue, or dermal tissue. In some embodiments, the promoter is an endogenous promoter of the cell. In some embodiments, the endogenous promoters of the cell drive the expression of one or more genes selected from Table 1.

In some embodiments, the non-coding region comprises one or more nucleases recognition sites. In some embodiments, at least one of the one or more nuclease recognition sites is selected from Table 3.

In some embodiments, the endogenous or exogenous nucleic acid is about 10 to about 700 bases in length, about 10 to about 600 bases, about 10 to about 500 bases, about 10 to about 400 bases, about 10 to about 300 bases, about 10 to about 200 bases in length, about 10 to about 180 bases, about 10 to about 160 bases, about 10 to about 140 bases, about 10 to about 120 bases, about 10 to about 110 bases, or about 10 to about 100 bases in length. In some embodiments, the endogenous or exogenous nucleic acid is less than 200 bases in length. In some embodiments, the endogenous or exogenous nucleic acid is positioned within the genome of the cell. In some embodiments, the endogenous or exogenous nucleic acid is not present on a plasmid.

In some embodiments, the endogenous or exogenous nucleic acid encodes a micro RNA (miRNA). In some embodiments, the miRNA is expressed as a short tandem target mimic (STTM) comprising two copies of partially complementary RNA linked by a spacer. In some embodiments, the spacer has a length of about 6 to about 60 nucleobases. In some embodiments, each of the two copies of partially complementary RNA have a length of about 10 to about 30 nucleobases. In some embodiments, the miRNA specifically binds to a target nucleic acid. In some embodiments, the target nucleic acid is exogenous to the cell. In some embodiments, the target nucleic acid is endogenous to the cell. In some embodiments, the target nucleic acid is responsible for water acquisition, nutrient acquisition, disease control, or pest control, or any combination thereof. In some embodiments, the target nucleic acid comprises a regulatory element involved in: plant growth and development, yield, biotic stress, abiotic stress, or herbicide resistance, or any combination thereof. In some embodiments, the target nucleic acid is from an insect, bacteria, fungi, worm (e.g., larva of the insect, and nematode), or a combination thereof, that is harmful to the cell. In some embodiments, the target nucleic acid is present in a target pest selected from Table 6. In some embodiments, the target nucleic acid is selected from the target genes in Table 6. In some embodiments, the target nucleic acid is from an organism that causes a disease to the cell. In some embodiments, the organism is any one selected from Table 6. In some embodiments, the target nucleic acid is a target mRNA. In some embodiments, the target mRNA comprises a sequence at least 70% identical to a sequence of Table 6. In some embodiments, the target mRNA is encoded from a target gene. In some embodiments, the target gene is selected from a gene shown in Table 6. In some embodiments, the target gene comprises a sequence at least 70% identical to a sequence of Table 6. In some embodiments, the endogenous or exogenous nucleic acid comprises a sequence at least 70% identical to a sequence of any one of the target gene sequences of Table 6, or the endogenous or exogenous nucleic acid comprises a sequence at least 80% identical to at least 10 contiguous bases of any one of the target gene sequences of Table 6.

In some embodiments, the endogenous or exogenous nucleic acid encodes a peptide. In some embodiments, the coding region for the peptide is flanked by a 5′ ribosomal binding site (RBS). In some embodiments, the RBS is 4-80 bases in length. In some embodiments, the peptide affects one or more biological functions of the cell selected from: hormonal regulation, protection against a pathogen, protection against an insect, nitrogen fixation, nutrient acquisition, immunity induction, biotic stress, or abiotic stress, or a combination thereof. In some embodiments, the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. In some embodiments, the peptide is selected from Table 7. In some embodiments, the peptide is encoded by a sequence at least 80% identical to a sequence of Table 8. In some embodiments, the endogenous or exogenous nucleic acid comprises a sequence at least 80% identical to a sequence of Table 8.

In another aspect, provided herein is a cell comprising an endogenous or exogenous micro RNA (miRNA). In some embodiments, the miRNA is endogenous to the cell but exogenous to the location of the miRNA in the cell. In some embodiments, the miRNA is exogenous to the cell. In some embodiments, the endogenous or exogenous miRNA harbors an artificial micro RNA (amiRNA). In some embodiments, the endogenous or exogenous miRNA is expressed as a short tandem target mimic (STTM) comprising two copies of partially complementary RNA linked by a spacer. In some embodiments, the spacer has a length of about 6 to about 60 nucleobases. In some embodiments, each of the two copies of partially complementary RNA have a length of about 10 to about 30 nucleobases. In some embodiments, the endogenous or exogenous miRNA is a precursor miRNA. In some embodiments, the endogenous or exogenous miRNA is a mature miRNA. In some embodiments, the mature miRNA comprises about 21-22 nucleotides. In some embodiments, the miRNA specifically binds to a target nucleic acid. In some embodiments, the target nucleic acid is endogenous or exogenous to the cell. In some embodiments, the target nucleic acid is endogenous to the cell. In some embodiments, the target nucleic acid is exogenous to the cell. In some embodiments, the target nucleic acid is responsible for water acquisition, nutrient acquisition, disease control, or pest control, or any combination thereof. In some embodiments, the target nucleic acid comprises a regulatory element involved in: plant growth and development, yield, biotic stress, abiotic stress, or herbicide resistance, or any combination thereof. In some embodiments, the target nucleic acid is from an insect, bacteria, fungi, nematode or a worm, or a combination thereof, that is harmful to the cell. In some embodiments, the target nucleic acid is present in a target pest selected from Table 6. In some embodiments, the target nucleic acid is selected from the target genes in Table 6. In some embodiments, the target nucleic acid is from an organism that causes a disease to the cell. In some embodiments, the organism is any one selected from Table 6. In some embodiments, the target nucleic acid is a target mRNA. In some embodiments, the target mRNA comprises a sequence at least 70% identical to a sequence of Table 6. In some embodiments, the target mRNA is encoded from a target gene. In some embodiments, the target gene is selected from a gene of Table 6. In some embodiments, the target gene comprises a sequence at least 70% identical to a sequence of Table 6.

In another aspect, provided herein is a cell comprising an endogenous or exogenous mRNA encoding a peptide. In some embodiments, the mRNA is endogenous to the cell but exogenous to the location of the mRNA in the cell. In some embodiments, the mRNA is exogenous to the cell. In some embodiments, the endogenous or exogenous mRNA is flanked by a 5′ribosomal binding site (RBS). In some embodiments, the RBS is 4-80 base pair in length. In some embodiments, the peptide affects one or more properties of the cell selected from: hormonal regulation, protection against a pathogen, protection against an insect, nitrogen fixation, nutrient acquisition, immunity induction, biotic stress, or abiotic stress, or a combination thereof. In some embodiments, the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. In some embodiments, the peptide is selected from Table 7. In some embodiments, the peptide is encoded by a sequence at least 80% identical to a sequence of Table 8. In some embodiments, the mRNA comprises a sequence at least 80% identical to a sequence of Table 8.

In another aspect, provided herein is a cell comprising an endogenous or exogenous peptide. In some embodiments, the peptide is endogenous to the cell. In some embodiments, the peptide is exogenous to the cell. In some embodiments, the peptide affects one or more properties of the cell, such as: hormonal regulation, protection against a pathogen, protection against an insect, nitrogen fixation, nutrient acquisition, immunity induction, biotic stress, or abiotic stress, and a combination of two or more thereof. In some embodiments, the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. In some embodiments, the peptide is selected from Table 7. In some embodiments, the peptide is encoded by a sequence at least 80% identical to a sequence of Table 8. In some embodiments, the cell is a plant cell. In some embodiments, the plant is a dicotyledonous plant. In some embodiments, the dicotyledonous plant is selected from Table 9. In some embodiments, the plant is a monocotyledonous plant. In some embodiments, the monocotyledonous plant is selected from Table 9. In some embodiments, the plant cell is a ground tissue cell. In some embodiments, the tissue cell is a parenchyma, collenchyma, or sclerenchyma cell. In some embodiments, the plant cell is a vascular tissue cell. In some embodiments, the tissue cell is a tracheid, vessel element, sieve tube cell, or companion cell. In some embodiments, the plant cell is a dermal tissue cell. In some embodiments, the tissue cell is an epidermal, guard cell, or trichome. In some embodiments, the cell is not transgenic. In some embodiments, the endogenous or exogenous nucleic acid is introduced into the cell via non-homologous recombination. In some embodiments, the endogenous or exogenous nucleic acid is introduced into the cell via non-homologous end-joining. In some embodiments, the endogenous or exogenous nucleic acid is introduced into the cell via homology-independent targeted integration (HITI). In some embodiments, the endogenous or exogenous nucleic acid is introduced into the cell via nuclease gene editing. In some embodiments, the nuclease gene editing comprises CRISPR-Cas gene editing.

In another aspect, provided herein is a host comprising any cell described herein. In some embodiments, the host is a plant. In some embodiments, the plant is a dicotyledonous plant. In some embodiments, the dicotyledonous plant is selected from Table 9. In some embodiments, the plant is a monocotyledonous plant. In some embodiments, the monocotyledonous plant is selected from Table 9. In some embodiments, the plant is not transgenic.

In another aspect, provided herein is a seed from any plant described herein.

In another aspect, provided herein is a plant obtained from any seed described herein.

In some embodiments, a plant described herein has one or more traits. In some embodiments, the one or more traits comprise hormonal regulation, protection against a pathogen, protection against an insect, nitrogen fixation, nutrient acquisition, immunity induction, biotic stress, or abiotic stress, or a combination thereof. In some embodiments, the trait is conferred by an endogenous or exogenous nucleic acid and/or peptide. In some embodiments, the endogenous or exogenous nucleic acid and/or peptide provides hormonal regulation, protection against a pathogen, protection against an insect, nitrogen fixation, nutrient acquisition, immunity induction, biotic stress, or abiotic stress, or a combination thereof. In some embodiments, the trait comprises resistance to a pest. In some embodiments, the pest is an insect, bacteria, fungi, worm (e.g., larva of the insect, and nematode), or a combination thereof. In some embodiments, the pest is selected from Table 6. In some embodiments, the resistance is due to antibiosis (growth and multiplication of the pest is inhibited), antixenosis (the pest is repelled by the plant), or tolerance (the plant is able to withstand or recover from damage by the pest). In some embodiments, the resistant plant has a superior yield as compared to a plant that does not comprise the cell with an endogenous or exogenous nucleic acid and/or peptide, when the plants are both under attack by the pest. In some embodiments, the trait comprises resistance to a disease. In some embodiments, the disease is caused by a pest. In some embodiments, the pest is an insect, bacteria, fungi, worm (e.g., larva of the insect, and nematode), or a combination thereof. In some embodiments, the pest is selected from Table 6. In some embodiments, the resistance is due to antibiosis (growth and multiplication of the pest is inhibited), antixenosis (the pest is repelled by the plant), or tolerance (plant is able to withstand or recover from damage by the pest). In some embodiments, the resistant plant has a superior yield as compared to a plant that does not comprise the cell with an exogenous nucleic acid and/or peptide, when the plants are both exposed to the disease. In some embodiments, the trait comprises resistance to a chemical. In some embodiments, the chemical is a weed control chemical. In some embodiments, the weed control chemical is a growth inhibitor. In some embodiments, the chemical is a herbicide. In some embodiments, the herbicide is 2,4-D (2,4-dichlorophenoxy acetic acid), Aminopyralid, Atrazine, Clopyralid, Dicamba, Glufosinate ammonium, Fluazifop, Fluroxypyr, Glyphosate, Imazapyr, Imazapic, Imazamox, Linuron, MCPA (2-methyl-4-chlorophenoxyacetic acid), Metolachlor, Paraquat, Pendimethalin, Picloram, Sodium chlorate, Triclopyr, Sulfonylureas (e.g., Flazasulfuron and Metsulfuron-methyl), or a combination thereof. In some embodiments, the trait confers an improved nutritional and/or visual quality as compared to a plant that does not comprise the cell with an exogenous nucleic acid and/or peptide, (e.g., measurable using a spectrometric method). In some embodiments, the trait confers an increase in crop yield as compared to a plant that does not comprise the cell with an exogenous nucleic acid and/or peptide. In some embodiments, the trait confers an ability to acquire a nutrient (e.g., nitrogen, phosphorus, potassium and/or plant micronutrients) at least 10% more efficiently as compared to a plant that does not comprise the cell with an exogenous nucleic acid and/or peptide (e.g., measurable using a spectrophotometric method). In some embodiments, the trait confers an ability to acquire water at least 10% more efficiently as compared to a plant that does not comprise the cell with an exogenous nucleic acid and/or peptide (e.g., measurable using the plant fresh weight when they were subjected to, for example, drought stress). In some embodiments, the trait confers at least 10% improved photosynthetic efficiency as compared to a plant that does not comprise the cell with an exogenous nucleic acid and/or peptide (e.g., measurable using, for example, a gas-exchange analyzer).

In another aspect, provided herein is a donor nucleic acid sequence comprising an endogenous or exogenous nucleic acid. In some embodiments, the endogenous or exogenous nucleic acid is exogenous to the donor nucleic acid sequence. In some embodiments, the endogenous or exogenous nucleic acid is about 10 to about 700 bases in length, about 10 to about 600 bases in length, about 10 to about 500 bases in length, about 10 to about 400 bases in length, about 10 to about 300 bases in length, about 10 to about 200 bases in length, about 10 to about 180 bases, about 10 to about 160 bases, about 10 to about 140 bases, about 10 to about 120 bases, about 10 to about 110 bases, or about 10 to about 100 bases in length. In some embodiments, the endogenous or exogenous nucleic acid is less than 200 bases in length. In some embodiments, the endogenous or exogenous nucleic acid encodes a micro RNA (miRNA). In some embodiments, the miRNA is expressed as a short tandem target mimic (STTM) comprising two copies of partially complementary RNA linked by a spacer. In some embodiments, the spacer has a length of about 6 to about 60 nucleobases. In some embodiments, each of the two copies of partially complementary RNA have a length of about 10 to about 30 nucleobases. In some embodiments, the miRNA specifically binds to a target nucleic acid. In some embodiments, the target nucleic acid is responsible for water acquisition, nutrient acquisition, disease control, or pest control, or any combination thereof. In some embodiments, the target nucleic acid comprises a regulatory element involved in plant growth and development, yield, biotic stress, abiotic stress, or herbicide resistance, or any combination thereof. In some embodiments, the target nucleic acid is from an insect, bacteria, fungi, worm (e.g., larva of the insect, and nematode), or a combination thereof, that is harmful to a cell. In some embodiments, the target nucleic acid is present in a target pest selected from Table 6. In some embodiments, the target nucleic acid is selected from the target genes in Table 6. In some embodiments, the target nucleic acid is from an organism that causes a disease to a cell. In some embodiments, the organism is any one selected from Table 6. In some embodiments, the target nucleic acid is a target mRNA. In some embodiments, the target mRNA comprises a sequence at least 70% identical to a sequence of Table 6. In some embodiments, the target mRNA is encoded from a target gene. In some embodiments, the target gene is selected from a gene of Table 6. In some embodiments, the target gene comprises a sequence at least 70% identical to a sequence of Table 6. In some embodiments, the endogenous or exogenous nucleic acid comprises a sequence at least 70% identical to a sequence of any one of the target gene sequences of Table 6, or the endogenous or exogenous nucleic acid comprises a sequence at least 80% identical to at least 10 contiguous bases of any one of the target gene sequences of Table 6. In some embodiments, the endogenous or exogenous nucleic acid encodes a peptide. In some embodiments, the coding region for the peptide is flanked by a 5′ribosomal binding site (RBS). In some embodiments, the RBS is 4-20 bases in length. In some embodiments, the peptide affects one or more properties of a cell selected from: hormonal regulation, protection against a pathogen, protection against an insect, nitrogen fixation, nutrient acquisition, immunity induction, biotic stress, or abiotic stress, or a combination thereof. In some embodiments, the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. In some embodiments, the peptide is selected from Table 7. In some embodiments, the peptide is encoded by a sequence at least 80% identical to a sequence of Table 8. In some embodiments, the endogenous or exogenous nucleic acid comprises a sequence at least 80% identical to a sequence of Table 8. In some embodiments, the donor nucleic acid is a blunt linear double-stranded oligodeoxynucleotide (dsODN). In some embodiments, the donor nucleic acid is a single-stranded oligodeoxynucleotide (ssODN). In some embodiments, the donor nucleic acid is a plasmid donor. In some embodiments, the donor nucleic acid comprises one or two nuclease recognition sites. In some embodiments, the donor nucleic acid comprises 2 nucleotides of phosphorothioate linkages at the 5′- and 3′-ends of both DNA strands of the exogenous nucleic acid. In some embodiments, the donor nucleic acid is phosphorylated at the 5′ end of both strands of the exogenous nucleic acid. In some embodiments, the non-coding region comprises an intron and the intron comprises the endogenous or exogenous nucleic acid. In some embodiments, the endogenous or exogenous nucleic acid is exogenous to the intron. In some embodiments, the non-coding region comprises a 5′ non-coding region, and the 5′ non-coding region comprises the endogenous or exogenous nucleic acid. In some embodiments, the endogenous or exogenous nucleic acid is exogenous to the 5′ non-coding region. In some embodiments, the non-coding region comprises a 3′ non-coding region, and the 3′ non-coding region comprises the endogenous or exogeneous nucleic acid. In some embodiments, the endogenous or exogenous nucleic acid is exogenous to the 3′ non-coding region.

Further provided is a kit comprising any donor nucleic acid herein, and a nucleic acid sequence encoding a DNA nuclease. In some embodiments, the DNA nuclease is as exemplified in Example 1. In some embodiments, the DNA nuclease is a CRISPR-associated nuclease. In some embodiments, the CRISPR-associated nuclease comprises Cas9. In some embodiments, the nucleic acid sequence encoding the DNA nuclease further encodes one or more guide RNA (gRNA). In some embodiments, the one or more gRNA are selected from Table 4. In some embodiments, the DNA nuclease is a Transcription Activator-Like Effector Nuclease (TALEN). In some embodiments, the DNA nuclease is connected to a sequence encoding VirD2 (e.g., Table 5). In some embodiments, the non-coding region comprises an intron and the intron comprises the endogenous or exogenous nucleic acid. In some embodiments, the endogenous or exogenous nucleic acid is exogenous to the intron. In some embodiments, the non-coding region comprises a 5′ non-coding region, and the 5′ non-coding region comprises the endogenous or exogenous nucleic acid. In some embodiments, the endogenous or exogenous nucleic acid is exogenous to the 5′ non-coding region. In some embodiments, the non-coding region comprises a 3′ non-coding region, and the 3′ non-coding region comprises the endogenous or exogenous nucleic acid. In some embodiments, the endogenous or exogenous nucleic acid is exogenous to the 3′ non-coding region.

Further provided is a combination comprising any donor nucleic acid herein, or kit herein, and a cell comprising an acceptor non-coding region for insertion of the donor nucleic acid sequence. In some embodiments, the endogenous or exogenous nucleic acid of the donor nucleic acid sequence is exogenous to the cell. In some embodiments, the endogenous or exogenous nucleic acid of the donor nucleic acid sequence is endogenous to the cell. In some embodiments, the cell is a plant cell. In some embodiments, the plant is a dicotyledonous plant. In some embodiments, the dicotyledonous plant is selected from Table 9. In some embodiments, the plant is a monocotyledonous plant. In some embodiments, the monocotyledonous plant is selected from Table 9. In some embodiments, the plant cell is a ground tissue cell. In some embodiments, the tissue cell is a parenchyma, collenchyma, or sclerenchyma cell. In some embodiments, the plant cell is a vascular tissue cell. In some embodiments, the tissue cell is a tracheid, vessel element, sieve tube cell, or companion cell. In some embodiments, the plant cell is a dermal tissue cell. In some embodiments, the tissue cell is an epidermal, guard cell, or trichome. In some embodiments, the cell is not transgenic. In some embodiments, the endogenous or exogenous nucleic acid is introduced into the cell via non-homologous recombination. In some embodiments, the endogenous or exogenous nucleic acid is introduced into the cell via non-homologous end-joining. In some embodiments, the endogenous or exogenous nucleic acid is introduced into the cell via homology-independent targeted integration (HITI). In some embodiments, the endogenous or exogenous nucleic acid is introduced into the cell via nuclease gene editing. In some embodiments, the nuclease gene editing comprises CRISPR-Cas gene editing. In some embodiments, the non-coding region comprises an intron and the intron comprises the endogenous or exogenous nucleic acid. In some embodiments, the endogenous or exogenous nucleic acid is exogenous to the intron. In some embodiments, the non-coding region comprises a 5′ non-coding region, and the 5′ non-coding region comprises the endogenous or exogenous nucleic acid. In some embodiments, the endogenous or exogenous nucleic acid is exogenous to the 5′ non-coding region. In some embodiments, the non-coding region comprises a 3′ non-coding region, and the 3′ non-coding region comprises the endogenous or exogeneous nucleic acid. In some embodiments, the endogenous or exogenous nucleic acid is exogenous to the 3′ non-coding region.

Further provided is a method of generating a cell with a modified (e.g., genetically edited) non-coding region, the method comprising introducing into the cell any donor nucleic acid herein, or any kit herein. In some embodiments, the modified (e.g., genetically edited) non-coding region comprises the endogenous or exogenous nucleic acid. Further provided is a method of generating a cell comprising a modified (e.g., genetically edited) non-coding region, the method comprising introducing an endogenous or exogenous nucleic acid into a non-coding of a gene in the cell. In some embodiments, the cell is a plant cell. In some embodiments, the endogenous or exogenous nucleic acid is introduced via non-homologous recombination. In some embodiments, the endogenous or exogenous nucleic acid is introduced via non-homologous end-joining. In some embodiments, the endogenous or exogenous nucleic acid is introduced via homology-independent targeted integration (HITI). In some embodiments, the endogenous or exogenous nucleic acid is introduced via nuclease gene editing. In some embodiments, the nuclease gene editing comprises CRISPR-Cas gene editing. In some embodiments, the endogenous or exogenous nucleic acid is exogenous to the donor nucleic acid. In some embodiments, the endogenous or exogenous nucleic acid is endogenous to the donor nucleic acid. In some embodiments, the endogenous or exogenous nucleic acid is exogenous to the cell. In some embodiments, the endogenous or exogenous nucleic acid is endogenous to the cell.

In another aspect, provided herein is a method of reducing or eliminating expression of a target gene in a cell, the method comprising introducing into a non-coding region of the cell an endogenous or exogenous nucleic acid, wherein the endogenous or exogenous nucleic acid encodes for a sequence that is capable of binding to mRNA of the target gene, thereby reducing or eliminating expression of the target gene. In some embodiments, the endogenous or exogenous nucleic acid is exogenous to the cell. In some embodiments, the endogenous or exogenous nucleic acid is endogenous to the cell.

In another aspect, provided herein is a method of regulating a target gene or peptide in a cell, the method comprising introducing, e.g., by gene editing, into a non-coding region of the cell an endogenous or exogenous nucleic acid, wherein the endogenous or exogenous nucleic acid encodes for an amino acid sequence that is capable of regulating the target gene or peptide in the cell, thereby regulating the target gene or peptide in the cell. In some embodiments, the endogenous or exogenous nucleic acid is exogenous to the cell. In some embodiments, the endogenous or exogenous nucleic acid is endogenous to the cell.

In another aspect, provided herein is a method of introducing, increasing, or reducing a trait in a host, the method comprising introducing, e.g., by gene editing, into a non-coding region of a cell of the host an endogenous or exogenous nucleic acid, wherein the endogenous or exogenous nucleic acid encodes for a sequence that is capable of binding to mRNA of a target gene, thereby introducing, increasing, or reducing a trait in the host. In some embodiments, the endogenous or exogenous nucleic acid is exogenous to the cell. In some embodiments, the endogenous or exogenous nucleic acid is endogenous to the cell.

In another aspect, provided herein is a method of introducing, increasing, or reducing a trait in a host, the method comprising introducing, e.g., by gene editing, into a non-coding region of a cell of the host an endogenous or exogenous nucleic acid, wherein the endogenous or exogenous nucleic acid encodes for an amino acid sequence that is capable of regulating a target gene or peptide in the cell, thereby introducing, increasing or reducing a trait in the host. In some embodiments, the endogenous or exogenous nucleic acid is exogenous to the cell. In some embodiments, the endogenous or exogenous nucleic acid is endogenous to the cell.

In some embodiments, the host is a plant. In some embodiments, the plant is a dicotyledonous plant. In some embodiments, the dicotyledonous plant is selected from Table 9. In some embodiments, the plant is a monocotyledonous plant. In some embodiments, the monocotyledonous plant is selected from Table 9. In some embodiments, the plant is not transgenic. In some embodiments, the trait comprises hormonal regulation, protection against a pathogen, protection against an insect, nitrogen fixation, nutrient acquisition, immunity induction, biotic stress, or abiotic stress, or a combination thereof. In some embodiments, the trait comprises resistance to a pest. In some embodiments, the pest is an insect, bacteria, fungi, worm (e.g., larva of the insect, and nematode), or a combination thereof. In some embodiments, the pest is selected from Table 6. In some embodiments, the resistance is due to antibiosis (growth and multiplication of the pest is inhibited), antixenosis (the pest is repelled by the plant), or tolerance (plant is able to withstand or recover from damage by the pest). In some embodiments, the host has a superior yield as compared to a host that does not comprise the endogenous or exogenous nucleic acid, when the hosts are both under attack by the pest. In some embodiments, the trait comprises resistance to a disease. In some embodiments, the disease is caused by a pest. In some embodiments, the pest is an insect, bacteria, fungi, worm (e.g., larva of the insect, and nematode), or a combination thereof. In some embodiments, the pest is selected from Table 6. In some embodiments, the resistance is due to antibiosis (growth and multiplication of the pest is inhibited), antixenosis (the pest is repelled by the plant), or tolerance (plant is able to withstand or recover from damage by the pest). In some embodiments, the resistant host has a superior yield as compared to a host that does not comprise the cell of any previous embodiment, when the hosts are both exposed to the disease. In some embodiments, the trait comprises resistance to a chemical. In some embodiments, the chemical is a weed control chemical. In some embodiments, the weed control chemical is a growth inhibitor. In some embodiments, the chemical is an herbicide. In some embodiments, the herbicide is 2,4-D (2,4-dichlorophenoxy acetic acid), Aminopyralid, Atrazine, Clopyralid, Dicamba, Glufosinate ammonium, Fluazifop, Fluroxypyr, Glyphosate, Imazapyr, Imazapic, Imazamox, Linuron, MCPA (2-methyl-4-chlorophenoxyacetic acid), Metolachlor, Paraquat, Pendimethalin, Picloram, Sodium chlorate, Triclopyr, Sulfonylureas (e.g., Flazasulfuron and Metsulfuron-methyl), or a combination thereof. In some embodiments, the trait confers an improved nutritional and/or visual quality as compared to a host that does not comprise the exogenous nucleic acid (e.g., measurable using a spectrometric method). In some embodiments, the trait confers an increase in crop yield as compared to a plant that does not comprise the exogenous nucleic acid. In some embodiments, the trait confers an ability to acquire a nutrient (e.g., nitrogen, phosphorus, potassium and/or plant micronutrients) at least 10% more efficiently as compared to a host that does not comprise the endogenous or exogenous nucleic acid (e.g., measurable using a spectrophotometric or spectrometric method). In some embodiments, the trait confers an ability to acquire water at least 10% more efficiently as compared to a host that does not comprise the endogenous or exogenous nucleic acid (e.g., measurable using the host fresh weight when they were subjected to, for example, drought stress). In some embodiments, the trait confers at least 10% improved photosynthetic efficiency as compared to a host that does not comprise the exogenous nucleic acid (e.g., measurable using, for example, a gas-exchange analyzer). In some embodiments, the endogenous or exogenous nucleic acid is about 10 to about 700 bases, about 10 to about 600 bases in length, about 10 to about 500 bases in length, about 10 to about 400 bases in length, about 10 to about 300 bases in length, about 10 to about 200 bases in length, about 10 to about 180 bases, about 10 to about 160 bases, about 10 to about 140 bases, about 10 to about 120 bases, about 10 to about 110 bases, or about 10 to about 100 bases in length. In some embodiments, the endogenous or exogenous nucleic acid is less than 200 bases in length. In some embodiments, the endogenous or exogenous nucleic acid encodes a micro RNA (miRNA). In some embodiments, the miRNA is expressed as a short tandem target mimic (STTM) comprising two copies of partially complementary RNA linked by a spacer. In some embodiments, the spacer has a length of about 6 to about 60 nucleobases. In some embodiments, each of the two copies of partially complementary RNA have a length of about 10 to about 30 nucleobases. In some embodiments, the miRNA specifically binds to a target nucleic acid. In some embodiments, the target nucleic acid is responsible for water acquisition, nutrient acquisition, disease control, or pest control, or any combination thereof. In some embodiments, the target nucleic acid comprises a regulatory element involved in: plant growth and development, yield, biotic stress, abiotic stress, or herbicide resistance, or any combination thereof. In some embodiments, the target nucleic acid is from an insect, bacteria, fungi, worm (e.g., larva of the insect, and nematode), or a combination thereof, that is harmful to a cell. In some embodiments, the target nucleic acid is present in a target pest selected from Table 6. In some embodiments, the target nucleic acid is selected from the target genes in Table 6. In some embodiments, the target nucleic acid is from an organism that causes a disease to a cell. In some embodiments, the organism is any one selected from Table 6. In some embodiments, the target nucleic acid is a target mRNA. In some embodiments, the target mRNA comprises a sequence at least 70% identical to a sequence of Table 6. In some embodiments, the target mRNA is encoded from a target gene. In some embodiments, the target gene is selected from a gene of Table 6. In some embodiments, the target gene comprises a sequence at least 70% identical to a sequence of Table 6. In some embodiments, the endogenous or exogenous nucleic acid comprises a sequence at least 70% identical to a sequence of any one of the target gene sequences of Table 6, or the endogenous or exogenous nucleic acid comprises a sequence at least 80% identical to at least 10 contiguous bases of any one of the target gene sequences of Table 6. In some embodiments, the endogenous or exogenous nucleic acid encodes a peptide. In some embodiments, the endogenous or exogenous nucleic acid is flanked by a 5′ribosomal binding site (RBS). In some embodiments, the RBS is 4-20 bases in length. In some embodiments, the peptide affects one or more property of a cell selected from: hormonal regulation, protection against a pathogen, protection against an insect, nitrogen fixation, nutrient acquisition, immunity induction, biotic stress, or abiotic stress, or a combination thereof. In some embodiments, the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. In some embodiments, the peptide is selected from Table 7. In some embodiments, the peptide is encoded by a sequence at least 80% identical to a sequence of Table 8. In some embodiments, the endogenous or exogenous nucleic acid comprises a sequence at least 80% identical to a sequence of Table 8. In some embodiments, the cell is a plant cell. In some embodiments, the plant is a dicotyledonous plant. In some embodiments, the dicotyledonous plant is selected from Table 9. In some embodiments, the plant is a monocotyledonous plant. In some embodiments, the monocotyledonous plant is selected from Table 9. In some embodiments, the plant cell is a ground tissue cell. In some embodiments, the tissue cell is a parenchyma, collenchyma, or sclerenchyma cell. In some embodiments, the plant cell is a vascular tissue cell. In some embodiments, the tissue cell is a tracheid, vessel element, sieve tube cell, or companion cell. In some embodiments, the plant cell is a dermal tissue cell. In some embodiments, the tissue cell is a epidermal, guard cell, or trichome. In some embodiments, the cell is not transgenic. In some embodiments, the endogenous or exogenous nucleic acid is exogenous to the cell. In some embodiments, the endogenous or exogenous nucleic acid is endogenous to the cell.

In any of the embodiments herein, the non-coding region comprises an intron and the intron comprises the endogenous or exogenous nucleic acid. In some embodiments, the endogenous or exogenous nucleic acid is exogenous to the intron. In any of the embodiments herein, the non-coding region comprises a 5′ non-coding region, and the 5′ non-coding region comprises the endogenous or exogenous nucleic acid. In some embodiments, the endogenous or exogenous nucleic acid is exogenous to the 5′ non-coding region. In any of the embodiments herein, the non-coding region comprises a 3′ non-coding region, and the 3′ non-coding region comprises the endogenous or exogeneous nucleic acid. In some embodiments, the endogenous or exogenous nucleic acid is exogenous to the 3′ non-coding region.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 Schematic representation of a non-limiting example of an intron editing platform for an amiRNA described herein. An amiRNA is a natural miRNA that had its natural 22 natural nucleotides replaced by an artificially designed 22 nucleotides. A) Genomic region of a constitutive and/or tissue-specific, highly expressed gene is selected to receive an insertion of the endogenous or exogenous nucleic acid into an intronic region. B) The endogenous or exogenous nucleic acid is an amiRNA inserted via genome editing. C) After transcription, subsequent splicing and amiRNA processing, the mature native gene mRNA and the mature amiRNA are produced. D) The native protein encoded by the genome edited gene is not affected in the genetically edited cell. E) Schematic representation of the genomic region of a target gene. F) After the amiRNA processing, the mature amiRNA silence the mRNA of the target gene.

FIG. 2 Schematic representation of a non-limiting example of an intron editing platform for a nucleic acid sequence encoding a small peptide described herein. A) Genomic region of a constitutive and/or tissue-specific, highly expressed gene is selected to receive an insertion of the endogenous or exogenous nucleic acid into an intronic region. B) The endogenous or exogenous nucleic acid encoding a small peptide is inserted via genome editing. C) After transcription, subsequent splicing and processing, the mature native gene mRNA and the mature mRNA encoding the small peptide are produced. D) The native protein encoded by the edited gene in A, is not affected in the engineered cell. E) After translation the mature small peptide performs different activities in the cell such as hormonal regulation, activity against a pathogen, activity against an inset, activity against a nematode, nitrogen fixation, nutrient acquisition, immunity induction, biotic stress, or abiotic stress, or a combination thereof.

FIG. 3 Schematic representation of a map of an example of a donor plasmid comprised of components including an endogenous or exogenous nucleic acid sequence encoding an amiRNA. The target gene for intron or non-coding region editing of a gene, for example, may be one selected from Table 1. The donor plasmid is prepared to deliver the amiRNA. The exemplified amiRNA is the ath-MIR172b. The amiRNA exemplified is flanked by the guide sequence 29rev from Os03t0718100-01 intron 1 of Table 4, in both sites (5′ and 3′ ends). The two guide sequences and PAM motif enable donor DNA release from the plasmid and insertion on the intron1 of the Actin1 in the rice host plant.

FIG. 4 Schematic representation of a map of an example of a binary plasmid comprised of components for CRISPR-Cas9 genome editing. The CRISPR-Cas9 plasmid contains one guide sequence such as the guide sequence 29rev from Os03t0718100-01 intron 1 of the Table 1.

FIG. 5 Schematic representation of a non-limiting example of donor DNA components including endogenous or exogenous nucleic acid sequence. A) Blunt single-stranded oligodeoxynucleotide (ssODN) (SEQ ID NO: 1528; GAATTCCGGCTCTCTACCGTCT), B) Blunt linear double-stranded oligodeoxynucleotide (dsODN) (SEQ ID NO: 1528 (GAATTCCGGCTCTCTACCGTCT), SEQ ID NO: 1529; AGACGGTAGAGAGCCGGAATTC), C) Chemically modified dsODN (dsODN-CM) (SEQ ID NO: 1530, SEQ ID NO: 1531). D) The donor DNA delivered as a plasmid. E) Donor plasmid cleaved by nuclease at S1 sites, releasing the donor fragment of endogenous or exogenous nucleic acid.

FIG. 6 Schematic representation of a non-limiting example of a method for preparing an engineered cell. A) Plasmid comprising a DNA sequence encoding a nuclease and a guide RNA. B) Donor plasmid with two specific Cas9 nuclease cleavage sites flanking the donor DNA comprising an endogenous or exogenous nucleic acid. C) Blunt linear double-stranded oligodeoxynucleotide (dsODN). D) Chemically modified dsODN (dsODN-CM). E) Blunt single-stranded oligodeoxynucleotide (ssODN). F) Scheme of an example of a selected gene to receive a donor DNA into an intron region of the gene. G) Scheme of nuclease mediated insertion of endogenous or exogenous nucleic acid into an intronic region via non-homologous end-joining. After splicing, the native functions of the H) gene and I) protein are preserved, and the amiRNA or the small peptide are produced. J) The amiRNA precursor is processed into a mature amiRNA that silences a target mRNA for a desired trait. K) Intron comprising a small peptide coding region to deliver a desired trait.

FIG. 7 Schematic representation of an exemplary embodiment where an amiRNA inserted by a platform described herein silences a target reporter gene in host-plant cells. A) Plasmid comprising a construct to transiently express an amiRNA inserted into an intron of a gene selected from Table 1. B) Plasmid comprising a construct to express a reporter gene into plant cells. C) Plasmids from A) and B), are simultaneously Agroinfiltrated in Nicotiana benthamiana leaves. D) Transient co-expression from plasmids described in A) and B).

FIG. 8 Exemplary experiment of Nicotiana benthamiana leaves Agroinfected with an Agrobacterium strain harboring plasmids as for example the ones represented in FIG. 7A and FIG. 7B. A) The top right leaf quadrant shows the Agroinfection with a control reporter construct. The expression of the reporter gene was visually observed. The top left leaf quadrant shows the co-Agroinfection with both the control reporter construct and a construct comprising an amiRNA designed to silence the reporter gene (positive control). The expression of the reporter gene was, visually, completely abolished. The bottom left leaf quadrant shows the co-Agroinfection with both the control reporter construct and a construct comprising an amiRNA (osaACT amiRNA-Reporter, SEQ ID NO: 1532 (TGATCATCTGGTCGTTGGCGT) designed to silence the reporter gene inserted into the intron 2 of the rice ACTIN gene (SEQ ID NO: 278). The expression of the reporter gene was, visually, completely abolished. The bottom right leaf quadrant shows the co-Agroinfection with the control reporter construct and a construct comprising an amiRNA (gmaACT amiRNA-Reporter, SEQ ID NO: 1532) designed to silence the reporter gene inserted into the intron 2 of the soybean ACTIN gene (SEQ ID NO: 533). The expression of the reporter gene was, visually, completely abolished. B) The amiRNA designed to silence the reporter gene accumulated in the bottom left and bottom right leaf quadrants indicating that the amiRNA inserted into the intron 2 of the actin genes from rice and soybean was correctly processed. C) The mRNA transcribed from the reporter gene were targeted and degraded by the amiRNA inserted into the intron 2 of the actin genes from rice and soybean. D) After transcription, splicing, amiRNA processing a mature ACTIN mRNA was produced. After translation, the correct, native ACTIN protein was produced.

FIG. 9 Schematic representation of an exemplary embodiment for the expression of a small peptide from an intron of a gene. A) A nucleic acid sequence encoding a small peptide embedded into an intron of the rice ACTIN. B) After Agroinfiltration in Nicotiana benthamiana leaves, the gene is transcribed, the mRNA processed and translated producing the small peptide involved, for example, in the plant hormonal signaling pathway. C) Plasmids from A) are transiently expressed in Nicotiana benthamiana leaves.

FIG. 10 Schematic representation of an example embodiment where a genetically edited plant described herein has a desirable trait as compared to a non-engineered plant. A) Schematic representation of the genomic region of an endogenous gene. Grey boxes (exons). Lines (introns). B) Schematic representation of processed amiRNA-Reporter. C) Schematic representation of Reporter gene silencing by the amiRNA-Reporter.

### DETAILED DESCRIPTION

In one aspect, the present disclosure relates to compositions and methods for the development of biotechnological traits, for instance, traits that increase crop quality and yield by making plants resistant to pests and diseases, plants resistant to weed control chemicals, such as herbicides, plants able to acquire nutrients and water in a more efficient manner, plants with improved photosynthetic efficiency, and fruits and seeds with improved qualities. Currently some of these agronomic useful traits are produced by engineering transgenic plants overexpressing gene constructs harboring resistance genes driven by strong and constitutive promoters. For example, insecticidal proteins from Bacillus thuringiensis are placed under the transcriptional control of strong constitutive promoters such as the 35S promoter from cauliflower mosaic virus, the actin promoter from rice, and the ubiquitin promoter from maize, among others. Such gene constructs are used to produced insect resistant transgenic crops. Strong and constitutive promoters occur in all living organisms and constitute part of the housekeeping genes encoding proteins and nucleic acids essential for all living cells. Significant parts of those housekeeping genes comprise genes that are expressed at very high levels. Examples of highly expressed housekeeping genes in eukaryotic organisms are the ones encoding actin, ubiquitin, ribosomal genes, genes encoding heat shock proteins, among others. The present disclosure describes a platform that uses non-coding regions, e.g., introns, 5′non-coding region and 3′non-coding regions, of said housekeeping genes that have been edited to express regulatory nucleic acids or peptides that, when expressed in a plant cell results in one or more desirable traits, e.g., traits that increase crop quality and yield by making plants resistant to pests and diseases, plants resistant to weed control chemicals, such as herbicides, plants able to acquire nutrients and water in a more efficient manner, plants with improved photosynthetic efficiency, and fruits and seeds with improved qualities.

In another aspect, the present disclosure relates to compositions and methods for the development of biotechnological traits that require tissue/organ specific expression of regulatory nucleic acids and/or small peptides. For example, there are biotechnological traits that require the use of root specific promoters, from highly expressed genes. Such root specific, high expression driven promoters are used to engineer traits related to resistance to root diseases, for example nematodes, among others. Other biotechnological traits may require, leaf specific promoters, fruit specific promoters, seed specific promoters, among others. The present disclosure describes a platform that uses the non-coding regions, e.g., introns, 5′non-coding region, and/or 3′non-coding regions, of said tissue/organ specific expression genes that have been edited to express regulatory nucleic acids that when expressed in a plant results in traits, such as those that increase crop quality and yield by making plants resistant to pests and diseases, plants resistant to weed control chemicals, such as herbicides, plants able to acquire nutrients and water in a more efficient manner, plants with improved photosynthetic efficiency, and fruits and seeds with improved quality.

In certain aspects, provided herein are platforms based on the insertion of DNA sequences into non-coding regions, e.g., introns, 5′non-coding region, and/or 3′non-coding regions, of constitutive and/or tissue-specific, highly expressed genes so that the inserted sequences, when transcribed, give rise to regulatory RNAs or mRNAs that, upon translation, give rise to regulatory peptides. In some embodiments these regulatory elements, when expressed constitutively and/or in a tissue-specific manner, result in useful traits to enhance quality and crop productivity. The insertion of DNA sequences into non-coding regions, e.g., introns, 5′non-coding region and 3′non-coding regions can be achieved by precision gene editing based on non-homologous end joining or any other molecular method allowing insertion of DNA sequences into non-coding regions, e.g., introns, 5′non-coding region and 3′non-coding regions through non-homologous recombination. The present disclosure provides a platform to deliver regulatory RNA, such as miRNA, and RNA molecules encoding regulatory elements that can be used for traits development in eukaryotic organisms such as plants, animals, and fungi.

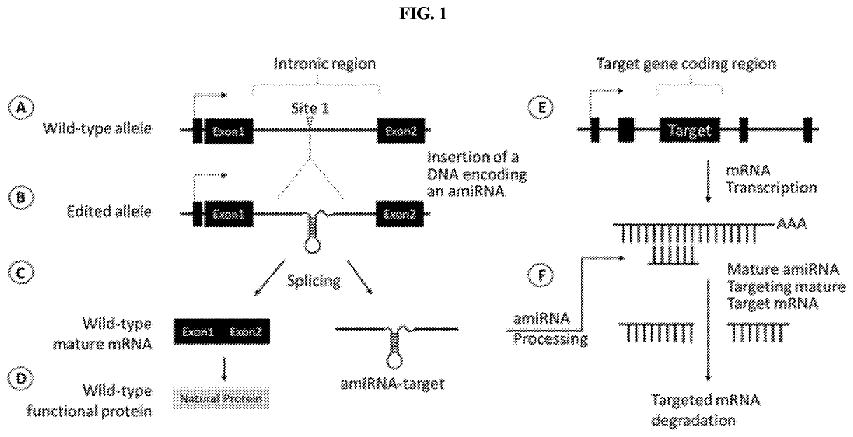

FIG. 1 shows a non-limiting example of a platform for amiRNA described herein. A) Scheme of genomic region of a host plant of the cell before splicing is shown. The natural allele (wild-type allele) of a constitutive and/or tissue-specific, highly expressed gene is designated to receive insertion of the endogenous or exogenous nucleic acid into a non-coding region exemplified as an intronic region. B) The endogenous or exogenous nucleic acid is an amiRNA inserted via genome editing using CRISPR-Cas9 technology and the endogenous DNA repair system non-homologous end joining. The insertion occurs in a single site of cleavage. C) After splicing, the post-splicing miRNA and the wild-type mature mRNA are present in the cell. D) The natural product of the genome edited gene in A is not affected in the engineered cell. E) Scheme of the genomic region of the target gene is shown. F) After the amiRNA processing, the mature amiRNA silences the target mRNA and the double stranded RNA are degraded by the cell machinery. The endogenous or exogenous nucleic acid may be exogenous to the cell. The endogenous or exogenous nucleic acid may be endogenous to the cell. The endogenous or exogenous nucleic acid may be exogenous to the non-coding region. The endogenous or exogenous nucleic acid may be endogenous to the non-coding region.

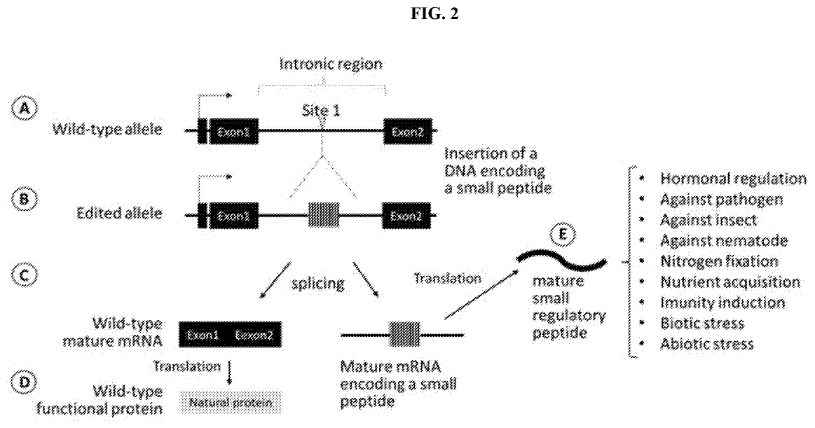

FIG. 2 shows a non-limiting example of a platform for small regulatory peptide described herein. A) Scheme of genomic region of a host plant of the cell before splicing is shown. The natural allele (wild-type allele) of a constitutive and/or tissue-specific, highly expressed gene is designated to receive an insertion of the endogenous or exogenous nucleic acid into a non-coding region exemplified as an intronic region. B) The endogenous or exogenous nucleic acid is a DNA encoding small peptide inserted via genome editing using CRISPR-Cas9 technology. The insertion is conducted by the endogenous DNA repair system non-homologous end joining. The insertion occurs in a single site of cleavage. C) After splicing, the post-splicing mature mRNA encoding a small peptide and the wild-type mature mRNA are present in the cell. D) The natural product of the genome edited gene in A, is not affected in the engineered cell. E) After processing (proteolyze and post-translational modifications), the mature small regulatory peptide regulates different processes in the cell. The endogenous or exogenous nucleic acid may be exogenous to the cell. The endogenous or exogenous nucleic acid may be endogenous to the cell. The endogenous or exogenous nucleic acid may be exogenous to the non-coding region. The endogenous or exogenous nucleic acid may be endogenous to the non-coding region.

### Cells

In one aspect, provided are cells comprising an endogenous or exogenous nucleic acid introduced into a non-coding region. In some examples, the non-coding region comprises an intron. In some examples, the non-coding region comprises a 5′ non-coding region (also referred to as a 5′ untranslated region or UTR). In some examples, the non-coding region comprises a 3′ non-coding region (also referred to as a 3′ UTR). Non-limiting components of such cells are described herein. The endogenous or exogenous nucleic acid may be exogenous to the cell. The endogenous or exogenous nucleic acid may be endogenous to the cell. The endogenous or exogenous nucleic acid may be exogenous to the non-coding region. The endogenous or exogenous nucleic acid may be endogenous to the non-coding region.

### Exons

Certain cells described herein comprise a first exon region and a second exon region. As used herein in certain embodiments, the first exon region and second exon region flank an intron that has been modified, and therefore the first exon region and second exon region are not limited to the first and second exons of a gene, and as shown in the examples herein, may represent the second and third exons of a gene, the third and fourth exons of a gene, and so on. In certain aspects, the first exon region and the second exon region are regions of a gene endogenous to the cell. Certain cells described herein comprise a 5′ non-coding region upstream of a gene endogenous to the cell. Certain cells described herein comprise a 3′ non-coding region downstream of a gene endogenous to the cell. In some embodiments, an exon region is adjacent to the 5′ non-coding region. In some embodiments, an exon region is adjacent to the 3′ non-coding region. In some embodiments, the gene endogenous to the cell is constitutively expressed. In one aspect, the gene endogenous to the cell is expressed in a specific tissue or organ. In some embodiments, the cell is a plant cell. Examples of the tissue or organ include, but not limited to, a root, stem, fruit, seed, leaf, ground tissue, vascular tissue, and dermal tissue.

In one aspect, the gene endogenous to the cell is highly expressed in the cell. In some embodiments, the expression of the gene endogenous to the cell corresponds to at least 1%, 2%, 3%, 4%, 5%, 6%, 7%, 8%, 9%, 10%, 11%, 12%, 13%, 14%, 15%, 16%, 17%, 18%, 19%, 20%, or more of the expression of all of the genes in the cell. In some embodiments, the expression of the gene endogenous to the cell is in the range of about 1-5%, 1-10%, 1-15%, 1-20%, 1-25%, 1-30%, 5-10%, 5-15%, 5-20%, 5-25%, 5-30%, 10-15%, 10-20%, 10-25%, 10-30%, 15-20%, 15-25%, 15-30%, 20-25%, 20-30%, 25-30% of the expression of all of the genes in the cell.

In one aspect, upon transcription and mRNA splicing, the native mRNA of the gene, e.g., a highly expressed gene, is translated into a native protein. In some embodiments, the gene encodes a native protein. Examples of the native protein include, but not limited to, actin, ubiquitin, ribosomal protein, heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, and the proteins encoded by the genes described in Table 1.

TABLE 1
Examples of genes encoding native proteins. The first column (SEQ ID NO) contains the sequence identifier of non-limiting
examples of genes encoding native proteins (SEQ ID NOS: 1-263). The second column (ORGANISM) describes the binomial scientific
name (genus and species) of non-limiting examples of organisms: Orysa sativa, rice; Glicine max, soybean; Hordeum vulgare,
barley; Solanum lycopersicum, tomato; Solanum tuberosum, potato; Sorghum bicolor, sorghum; Triticum aestivum,
wheat; Zea mays, maize. The third column (ENSEMBL IDENTIFIER) contains the code identifier of the gene deposited in
EnsemblPlants database (https://plants.ensembl.org/index.html). The fourth column (NCBI GENE ID) contains each code to
the NCBI gene identifier. A person of skill in the art would be able to search the NCBI database with such value and
retrieve information of the gene, including expression information. The fifth column (FASTA SEQUENCE) contains the NCBI
Reference Sequence Identifier. The FASTA sequence is available in the corresponding sequence listing filed with the present
application. The sixth column (GENE NAME) describes the name of the correspondent sequence.

| SEQ | | | | FASTA | |
|---|---|---|---|---|---|
| ID NO | ORGANISM | Ensembl identifier | NCBI GENE ID | SEQUENCE | GENE NAME |
| 1 | Oryza sativa | Os03g0718100 | LOC4333919 | NC_029258.1 | actin 1 |
| 2 | Oryza sativa | Os05g0106600 | LOC4337566 | NC_029260.1 | actin 97 |
| 3 | Oryza sativa | Os11g0163100 | LOC4349863 | NC_029266.1 | actin-7 |
| 4 | Oryza sativa | Os03g0836000 | LOC4334702 | NC_029258.1 | actin-3 |
| 5 | Oryza sativa | Os01g0964133 | LOC9269066 | NC_029256.1 | actin-97 |
| 6 | Oryza sativa | Os10g0510000 | LOC4349087 | NC_029265.1 | actin-2 |
| 7 | Oryza sativa | Os12g0163700 | LOC4351585 | NC_029267.1 | actin 7 |
| 8 | Oryza sativa | Os05g0438800 | LOC4338914 | NC_029260.1 | actin 1 |
| 9 | Oryza sativa | Os01g0866100 | LOC4325068 | NC_029256.1 | actin-7 |
| 10 | Oryza sativa | Os03g0783000 | LOC9266759 | NC_029258.1 | actin-7 |
| 11 | Oryza sativa | Os08g0137200 | LOC4344621 | NC_029263.1 | actin-4 |
| 12 | Oryza sativa | Os02g0596900 | LOC4329867 | NC_029257.1 | actin-3 |
| 13 | Oryza sativa | Os08g0369300 | LOC4345402 | NC_029263.1 | actin-2 |
| 14 | Oryza sativa | Os01g0269900 | LOC4326270 | NC_029256.1 | actin-6 |
| 15 | Oryza sativa | Os04g0177600 | LOC4335089 | NC_029259.1 | actin-9 |
| 16 | Oryza sativa | Os01g0144340 | LOC107275681 | NC_029256.1 | actin-5 |
| 17 | Oryza sativa | Os04g0667700 | LOC4337330 | NC_029259.1 | actin-8 |
| 18 | Glycine max | GLYMA_04G215900 | LOC100789000 | NC_016091.4 | actin-7 |
| 19 | Glycine max | GLYMA_07G118800 | LOC100795390 | NC_038243.2 | actin-4 |
| 20 | Glycine max | GLYMA_02G091900 | LOC100781831 | NC_016089.4 | actin |
| 21 | Glycine max | GLYMA_18G290800 | LOC100792119 | NC_038254.2 | actin-6 |
| 22 | Glycine max | GLYMA_05G000900 | LOC100798523 | NM_001253024.2 | actin-like |
| 23 | Glycine max | GLYMA_19G000900 | LOC100799890 | NC_038255.2 | actin-97 |
| 24 | Glycine max | GLYMA_15G038700 | LOC100815472 | NC_038251.2 | actin |
| 25 | Glycine max | GLYMA_11G139800 | LOC100811630 | NC_038247.2 | actin |
| 26 | Glycine max | GLYMA_13G335600 | LOC100787265 | NC_038249.2 | actin |
| 27 | Glycine max | GLYMA_19G147900 | LOC100807341 | NC_038255.2 | actin |
| 28 | Glycine max | GLYMA_09G111200 | LOC100777705 | NC_038245.2 | actin |
| 29 | Glycine max | GLYMA_03G144800 | LOC100781142 | NC_016090.4 | actin-3 |
| 30 | Glycine max | GLYMA_02G172800 | LOC100813210 | NC_016089.4 | actin |
| 31 | Glycine max | GLYMA_04G215900 | LOC100789000 | NC_016091.4 | actin-7 |
| 32 | Glycine max | GLYMA_15G050200 | LOC100778206 | NC_038251.2 | actin-101 |
| 33 | Glycine max | GLYMA_08G182200 | LOC100797704 | NC_038244.2 | actin-101 |
| 34 | Glycine max | GLYMA_12G63400 | LOC100813437 | NC_038248.2 | actin |
| 35 | Glycine max | GLYMA_05G067600 | LOC106798766 | NC_038241.2 | actin-46 |
| 36 | Glycine max | GLYMA_19G095900 | LOC100803980 | NC_038255.2 | actin-2 |
| 37 | Glycine max | GLYMA_16G053400 | LOC100796648 | NC_038252.2 | actin-2 |
| 38 | Glycine max | GLYMA_15G275500 | LOC100794153 | NC_038251.2 | actin-7 |
| 39 | Glycine max | GLYMA_09G229000 | LOC100814978 | NC_038245.2 | actin-7 |
| 40 | Glycine max | GLYMA_12G007600 | LOC100813266 | NC_038248.2 | actin-7 |
| 41 | Glycine max | GLYMA_10G089200 | LOC100803056 | NC_038246.2 | actin-7 |
| 42 | Glycine max | GLYMA_03G107300 | LOC100785066 | NC_016090.4 | actin-4 |
| 43 | Glycine max | GLYMA_07G118800 | LOC100795390 | NC_038243.2 | actin-4 |
| 44 | Glycine max | GLYMA_08G040300 | LOC100819549 | NC_038244.2 | actin-4 |
| 45 | Glycine max | GLYMA_05G172200 | LOC100778870 | NC_038241.2 | actin-3 |
| 46 | Glycine max | GLYMA_05G172600 | LOC100780462 | NC_038241.2 | actin-3 |
| 47 | Glycine max | GLYMA_04G071500 | LOC100819827 | NC_016091.4 | actin-6 |
| 48 | Glycine max | GLYMA_05G232900 | LOC100778866 | NC_038241.2 | actin-4 |
| 49 | Glycine max | GLYMA_16G096700 | LOC100804254 | NC_038252.2 | actin-8 |
| 50 | Glycine max | GLYMA_06G248100 | LOC100809724 | NC_038242.2 | actin-4a |
| 51 | Glycine max | GLYMA_14G067800 | LOC100790295 | NC_038250.2 | actin-5 |
| 52 | Glycine max | GLYMA_04G112600 | LOC100791973 | NC_016091.4 | actin-8 |
| 53 | Glycine max | GLYMA_11G219700 | LOC100813421 | NC_038247.2 | actin-5 |
| 54 | Glycine max | GLYMA_18G037700 | LOC100808396 | NC_038254.2 | actin-5 |
| 55 | Glycine max | GLYMA_02G248700 | LOC100804305 | NC_016089.4 | actin-5 |
| 56 | Glycine max | GLYMA_20G202700 | LOC100788484 | NC_038256.2 | actin-9 |
| 57 | Glycine max | GLYMA_10G188100 | LOC100797938 | NC_038246.2 | actin-9 |
| 58 | Hordeum vulgare | HORVU.MOREX.r3.4HG0337850 | LOC123447531 | NC_058521.1 | actin-1 |
| 59 | Hordeum vulgare | HORVU.MOREX.r3.5HG0419480 | LOC123399877 | NC_058522.1 | actin-1-like |
| 60 | Hordeum vulgare | HORVU.MOREX.r3.5HG053200 | LOC123394939 | NC_058522.1 | actin-3 |
| 61 | Hordeum vulgare | HORVU.MOREX.r3.5HG0457850 | LOC123398932 | NC_058522.1 | actin-3-like |
| 62 | Hordeum vulgare | HORVU.MOREX.r3.1HG0003140 | LOC123430406 | NC_058518.1 | actin-97-like |
| 63 | Hordeum vulgare | HORVU.MOREX.r3.1HG0049980 | LOC123434401 | NC_058518.1 | actin-2 |
| 64 | Hordeum vulgare | HORVU.MOREX.r3.1HG0075220 | LOC123446308 | NC_058518.1 | actin-7 |
| 65 | Hordeum vulgare | HORVU.MOREX.r3.3HG0299830 | LOC123444811 | NC_058520.1 | actin-7-like |
| 66 | Hordeum vulgare | HORVU.MOREX.r3.4HG0336970 | LOC123447433 | NC_058521.1 | actin-related protein 2 |
| 67 | Hordeum vulgare | HORVU.MOREX.r3.5HG0515940 | LOC123452545 | NC_058522.1 | actin-related protein 7 |
| 68 | Hordeum vulgare | HORVU.MOREX.r3.5HG0504450 | LOC123451923 | NC_058522.1 | actin-related protein 7 |
| 69 | Hordeum vulgare | HORVU.MOREX.r3.6HG0572330 | LOC123401347 | NC_058523.1 | actin-related protein 4 |
| 70 | Hordeum vulgare | HORVU.MOREX.r3.6HG0591310 | LOC123402112 | NC_058523.1 | actin-related protein 3 |
| 71 | Hordeum vulgare | HORVU.MOREX.r3.7HG0703200 | LOC123409811 | NC_058524.1 | actin-related protein 3-like |
| 72 | Hordeum vulgare | HORVU.MOREX.r3.2HG0213240 | LOC123428445 | NC_058519.1 | actin-related protein 6 |
| 73 | Hordeum vulgare | HORVU.MOREX.r3.2HG0208800 | LOC123428295 | NC_058519.1 | actin-related protein 8 |
| 74 | Hordeum vulgare | HORVU.MOREX.r3.3HG0229480 | LOC123442658 | NC_058520.1 | actin-related protein 5 |
| 75 | Hordeum vulgare | HORVU.MOREX.r3.2HG0099270 | LOC123424374 | NC_058519.1 | Actin-related protein 9 |
| 76 | Solanum lycoperscium | Solyc10g086460.2 | LOC101255046 | NC_015447.3 | actin-75 |
| 77 | Solanum lycoperscium | Solyc04g011500.3 | LOC101260631 | NC_015441.3 | actin |
| 78 | Solanum lycoperscium | Solyc10g080500.2 | LOC101263261 | NC_015447.3 | actin |
| 79 | Solanum lycoperscium | Solyc03g078400.3 | LOC101264601 | NC_015440.3 | actin-7 |
| 80 | Solanum lycopersicum | Solyc11g065990.2 | LOC101253966 | NC_015448.3 | actin-97 |
| 81 | Solanum lycopersicum | Solyc06g076090.3 | LOC101249734 | NC_015443.3 | actin-82 |
| 82 | Solanum lycopersicum | Solyc00g017210.2 | LOC101253675 | NW_020442571.1 | actin-1 |
| 83 | Solanum lycopersicum | Solyc01g104775.1 | LOC101250165 | NC_015438.3 | actin |
| 84 | Solanum lycopersicum | Solyc10g086460.2 | LOC101255046 | NC_015447.3 | actin-75 |
| 85 | Solanum lycopersicum | Solyc04g071260.3 | LOC101264618 | NC_015441.3 | actin-105 |
| 86 | Solanum lycopersicum | Solyc09g010750.2 | LOC101255728 | NC_015446.3 | actin-71 |
| 87 | Solanum lycopersicum | Solyc11g005330.2 | LOC101262163 | NC_015448.3 | actin-7 |
| 88 | Solanum lycopersicum | Solyc12g037980.2 | LOC101260909 | NC_015449.3 | actin-4 |
| 89 | Solanum lycopersicum | Solyc04g024530.3 | LOC101252768 | NC_015441.3 | actin-3 |
| 90 | Solanum lycopersicum | Solyc05g013940.3 | LOC101250599 | NC_015442.3 | actin-3 |
| 91 | Solanum lycopersicum | Solyc05g018600.3 | LOC101245946 | NC_015442.3 | actin-6 |
| 92 | Solanum lycopersicum | Solyc07g066120.3 | LOC101264034 | NC_015444.3 | actin-8 |
| 93 | Solanum lycopersicum | Solyc06g043175.1 | LOC101260649 | NC_015443.3 | actin-5 |
| 94 | Solanum lycopersicum | Solyc09g089660.3 | LOC101244230 | NC_015446.3 | actin-9 |
| 95 | Solanum tuberosum | PGSC0003DMG400023708 | LOC102597225 | NW_006239057.1 | actin-66 |
| 96 | Solanum tuberosum | PGSC0003DMG400000439 | LOC102590523 | NW_006239029.1 | actin-65 |
| 97 | Solanum tuberosum | PGSC0003DMG400030319 | LOC102593904 | NW_006239053.1 | actin-82 |
| 98 | Solanum tuberosum | PGSC0003DMG400023708 | LOC102597225 | NW_006239057.1 | actin-66 |
| 99 | Solanum tuberosum | PGSC0003DMG400023429 | LOC102582178 | NW_006238999.1 | actin-58 |
| 100 | Solanum tuberosum | PGSC0003DMG400027746 | LOC102577777 | NW_006239491.1 | actin-97 |
| 101 | Solanum tuberosum | PGSC0003DMG400003985 | LOC102599168 | NW_006239054.1 | actin-7 |
| 102 | Solanum tuberosum | PGSC0003DMG400018449 | LOC102584969 | NW_006239115.1 | actin-7 |
| 103 | Solanum tuberosum | PGSC0003DMG400029745 | LOC102593148 | NW_006239061.1 | actin-101 |
| 104 | Solanum tuberosum | PGSC0003DMG400019204 | LOC102601944 | NW_006239032.1 | actin-75 |
| 105 | Solanum tuberosum | PGSC0003DMG400008912 | LOC102606253 | NW_006238947.1 | actin-71 |
| 106 | Solanum tuberosum | PGSC0003DMG400029746 | LOC102593148 | NW_006239061.1 | actin-101 |
| 107 | Solanum tuberosum | PGSC0003DMG400008619 | LOC102592284 | NW_006239231.1 | actin-104 |
| 108 | Solanum tuberosum | PGSC0003DMG400008618 | LOC102592628 | NW_006239231.1 | actin-100 |
| 109 | Solanum tuberosum | PGSC0003DMG400029120 | LOC102594941 | NW_006239231.1 | actin-100 |
| 110 | Solanum tuberosum | PGSC0003DMG400029121 | LOC102594616 | NW_006239231.1 | actin-104 |
| 111 | Solanum tuberosum | PGSC0003DMG400020244 | LOC102583119 | NW_006238930.1 | actin-2 |
| 112 | Solanum tuberosum | PGSC0003DMG402007428 | LOC102598577 | NW_006239290.1 | actin-7 |
| 113 | Solanum tuberosum | PGSC0003DMG400021766 | LOC102600647 | NW_006239317.1 | actin-4 |
| 114 | Solanum tuberosum | PGSC0003DMG400010772 | LOC102600427 | NW_006238953.1 | actin-3 |
| 115 | Solanum tuberosum | PGSC0003DMG400014966 | LOC102596585 | NW_006238929.1 | actin-6 |
| 116 | Solanum tuberosum | PGSC0003DMG400022148 | LOC102593881 | NW_006239000.1 | actin-8 |
| 117 | Solanum tuberosum | PGSC0003DMG400017256 | LOC102601622 | NW_006239103.1 | actin-9 |
| 118 | Sorghum bicolor | SORBI_3005G047100 | LOC8083089 | NC_012874.2 | actin-7 |
| 119 | Sorghum bicolor | SORBI_3001G197400 | LOC8065178 | NC_012870.2 | actin-2 |
| 120 | Sorghum bicolor | SORBI_3009G006100 | LOC8068648 | NC_012878.2 | actin-97 |
| 121 | Sorghum bicolor | SORBI_3008G047000 | LOC110429756 | NC_012877.2 | actin-7 |
| 122 | Sorghum bicolor | SORBI_3001G022800 | LOC8080194 | NC_012870.2 | actin-3 |
| 123 | Sorghum bicolor | SORBI_3003G367300 | LOC8062375 | NC_012872.2 | actin-7 |
| 124 | Sorghum bicolor | SORBI_3009G153000 | LOC8058524 | NC_012878.2 | actin-1 |
| 125 | Sorghum bicolor | SORBI_3009G005900 | LOC110430012 | NC_012878.2 | actin-97 |
| 126 | Sorghum bicolor | SORBI_3002G426300 | LOC8077510 | NC_012871.2 | actin-2 |
| 127 | Sorghum bicolor | SORBI_3001G234200 | LOC8059297 | NC_012870.2 | actin-7 |
| 128 | Sorghum bicolor | SORBI_3001G536000 | LOC8078015 | NC_012870.2 | actin-4 |
| 129 | Sorghum bicolor | SORBI_3004G203500 | LOC110434588 | NC_012873.2 | actin-3 |
| 130 | Sorghum bicolor | SORBI_3007G026800 | LOC8066138 | NC_012876.2 | actin-3 |
| 131 | Sorghum bicolor | SORBI_3008G173100 | LOC8071478 | NC_012877.2 | actin-6 |
| 132 | Sorghum bicolor | SORBI_3006G257800 | LOC8070289 | NC_012875.2 | actin-8 |
| 133 | Sorghum bicolor | SORBI_3003G074200 | LOC8073310 | NC_012872.2 | actin-5 |
| 134 | Sorghum bicolor | SORBI_3006G029100 | LOC8067782 | NC_012875.2 | actin-9 |
| 135 | Zea mays | Zm00001eb222460 | LOC100193210 | NC_050100.1 | arp7 - actin related protein |
| 136 | Zea mays | Zm00001eb366720 | LOC100281811 | NC_050103.1 | actin 2 |
| 137 | Zea mays | Zm00001eb246220 | LOC100281189 | NC_050100.1 | no description |
| 138 | Zea mays | Zm00001eb216070 | LOC103625937 | NC_050100.1 | actin-1 |
| 139 | Zea mays | Zm00001eb202400 | LOC100283878 | NC_050099.1 | no description |
| 140 | Zea mays | Zm00001eb086290 | LOC103646627 | NC_050097.1 | actin |
| 141 | Zea mays | Zm00001eb06540 | LOC103644169 | NC_050096.1 | no description |
| 142 | Zea mays | Zm00001eb267280 | LOC103629276 | NC_050101.1 | act97 |
| 143 | Zea mays | Zm00001eb092070 | LOC100304239 | NC_050097.1 | no description |
| 144 | Zea mays | Zm00001eb267260 | LOC103629275 | NC_050101.1 | act-97 |
| 145 | Zea mays | Zm00001eb220480 | LOC100280540 | NC_050100.1 | act-2 |
| 146 | Zea mays | Zm00001eb043800 | LOC100381643 | NC_050096.1 | actin |
| 147 | Zea mays | Zm00001eb063720 | LOC100273404 | NC_050096.1 | no description |
| 148 | Zea mays | Zm00001eb366720 | LOC100281811 | NC_050103.1 | actin 2 |
| 149 | Zea mays | Zm00001eb079680 | LOC103646315 | NC_050097.1 | actin |
| 150 | Zea mays | Zm00001eb146780 | LOC100273396 | NC_050098.1 | actin-7 |
| 151 | Zea mays | Zm00001eb242040 | LOC103628660 | NC_050100.1 | actin-7 |
| 152 | Zea mays | Zm00001eb356050 | LOC103635981 | NC_050103.1 | actin-97 |
| 153 | Zea mays | Zm00001eb055330 | LOC100284092 | NC_050096.1 | no description |
| 154 | Zea mays | Zm00001eb348450 | LOC100282267 | NC_050103.1 | actin-1 |
| 155 | Zea mays | Zm00001eb331340 | LOC103633595 | NC_050102.1 | actin-2 |
| 156 | Zea mays | Zm00001eb000800 | LOC100279759 | NC_050096.1 | no description |
| 157 | Zea mays | Zm00001eb183860 | LOC100384280 | NC_050099.1 | actin-3 |
| 158 | Zea mays | Zm00001eb261670 | LOC100281262 | NC_050101.1 | no description |
| 159 | Zea mays | Zm00001eb173290 | LOC100192466 | NC_050099.1 | no description |
| 160 | Zea mays | Zm00001eb411820 | LOC100284728 | NC_050105.1 | no description |
| 161 | Zea mays | Zm00001eb335830 | LOC100383901 | NC_050103.1 | no description |
| 162 | Glycine max | GLYMA_19G253300 | LOC100786327 | NC_038255.2 | tubulin gamma-2 chain |
| 163 | Glycine max | GLYMA_19G127700 | LOC100798849 | NC_038255.2 | tubulin beta-4 |
| 164 | Glycine max | GLYMA_16G154000 | LOC100780531 | NC_038252.2 | tubulin alpha-3 |
| 165 | Glycine max | GLYMA_11G044200 | LOC100785622 | NC_038247.2 | tubulin alpha-2 |
| 166 | Glycine max | GLYMA_19G113000 | LOC100796371 | NC_038255.2 | tubulin alpha-6 |
| 167 | Glycine max | GLYMA_03G124400 | LOC547844 | NC_016090.4 | beta-tubulin |
| 168 | Glycine max | GLYMA_17G258300 | LOC100819408 | NC_038253.2 | tubulin beta |
| 169 | Glycine max | GLYMA_10G235100 | LOC100788253 | NC_038246.2 | tubulin beta-1 |
| 170 | Glycine max | GLYMA_09G026100 | LOC100818878 | NC_038245.2 | tubulin beta |
| 171 | Glycine max | GLYMA_01G109300 | LOC100816898 | NC_016088.4 | tubulin beta-2 |
| 172 | Glycine max | GLYMA_10G255500 | LOC100799688 | NC_038246.2 | tubulin alpha |
| 173 | Glycine max | GLYMA_03G255800 | LOC100775439 | NC_016090.4 | tubulin gamma-1 |
| 174 | Glycine max | GLYMA_06G090500 | LOC100807401 | NC_038242.2 | tubulin alpha-1 |
| 175 | Glycine max | GLYMA_05G110200 | LOC100787058 | NC_038241.2 | tubulin alpha-3 |
| 176 | Glycine max | GLYMA_04G088500 | LOC100779027 | NC_016091.4 | tubulin alpha-1 |
| 177 | Glycine max | GLYMA_05G126100 | LOC100784236 | NC_038241.2 | tubulin beta |
| 178 | Glycine max | GLYMA_08G081100 | LOC100801608 | NC_038244.2 | tubulin beta |
| 179 | Glycine max | GLYMA_01G197500 | LOC100786598 | NC_016088.4 | tubulin alpha-2 |
| 180 | Glycine max | GLYMA_16G040100 | LOC100784487 | NC_038252.2 | tubulin alpha-3 |
| 181 | Glycine max | GLYMA_20G136000 | LOC100781185 | NC_038256.2 | tubulin alpha-4 |
| 182 | Glycine max | GLYMA_05G207500 | LOC100797652 | NC_038241.2 | tubulin beta-1 |
| 183 | Glycine max | GLYMA_04G023900 | LOC100781525 | NC_016091.4 | tubulin beta |
| 184 | Glycine max | GLYMA_20G159200 | LOC100793406 | NC_038256.2 | tubulin beta-1 |
| 185 | Oryza sativa | Os03g0726100 | LOC4333966 | NC_029258.1 | alpha-1 tubulin |
| 186 | Oryza sativa | Os03g0105600 | LOC4331315 | NC_029258.1 | tubulin beta-2 |
| 187 | Oryza sativa | Os02g0167300 | LOC4328420 | NC_029257.1 | tubulin beta-5 |
| 188 | Oryza sativa | Os05g0156600 | LOC4337861 | NC_029260.1 | tubulin gamma-2 |
| 189 | Oryza sativa | Os01g0282800 | LOC4326917 | NC_029256.1 | tubulin beta-1 |
| 190 | Oryza sativa | Os03g0219300 | LOC4332083 | NC_029258.1 | alpha-2 tubulin |
| 191 | Oryza sativa | Os07g0574800 | LOC4343694 | NC_029262.1 | tubulin alpha-1 |
| 192 | Oryza sativa | Os06g0671900 | LOC4341810 | NC_029261.1 | tubulin beta-3 |
| 193 | Oryza sativa | Os03g0780600 | LOC4334309 | NC_029258.1 | tubulin beta-7 |
| 194 | Oryza sativa | Os05g0413200 | LOC4338790 | NC_029260.1 | tubulin beta-6 |
| 195 | Oryza sativa | Os03g0661300 | LOC4333632 | NC_029258.1 | tubulin beta-8 |
| 196 | Oryza sativa | Os11g0247300 | LOC4350197 | NC_029266.1 | tubulin alpha-2 |
| 197 | Sorghum bicolor | SORBI_3003G328800 | LOC8082412 | NC_012872.2 | tubulin beta-4 chain |
| 198 | Sorghum bicolor | SORBI_3009G052100 | LOC8071186 | NC_012878.2 | tubulin gamma-2 chain |
| 199 | Sorghum bicolor | SORBI_3004G053300 | LOC8076107 | NC_012873.2 | tubulin beta chain |
| 200 | Sorghum bicolor | SORBI_3001G540900 | LOC8059525 | NC_012870.2 | tubulin beta-1 chain |
| 201 | Sorghum bicolor | SORBI_3001G073700 | LOC8083952 | NC_012870.2 | tubulin alpha-3 chain |
| 202 | Sorghum bicolor | SORBI_3001G453700 | LOC8056877 | NC_012870.2 | tubulin alpha-2 chain |
| 203 | Sorghum bicolor | SORBI_3001G146000 | LOC8057201 | NC_012870.2 | tubulin beta-3 chain |
| 204 | Sorghum bicolor | SORBI_3001G069800 | LOC8084157 | NC_012870.2 | tubulin beta-7 chain |
| 205 | Sorghum bicolor | SORBI_3010G224900 | LOC8061601 | NC_012879.2 | tubulin beta-7 chain |
| 206 | Solanum lycoperscium | Solyc06g035970.3 | LOC101265829 | NC_015443.3 | tubulin beta-1 chain |
| 207 | Solanum lycoperscium | Solyc04g077020.3 | LOC101244864 | NC_015441.3 | tubulin alpha chain |
| 208 | Solanum lycoperscium | Solyc03g111380.3 | LOC101260712 | NC_015440.3 | tubulin gamma chain |
| 209 | Solanum lycoperscium | Solyc03g025730.3 | LOC101251552 | NC_015440.3 | tubulin beta chain |
| 210 | Solanum lycoperscium | Solyc10g086760.2 | LOC101252240 | NC_015447.3 | tubulin beta-2 chain |
| 211 | Solanum lycoperscium | Solyc08g006890.3 | LOC101254013 | NC_015445.3 | tubulin alpha-3 chain |
| 212 | Solanum lycoperscium | Solyc04g081490.3 | LOC778227 | NC_015441.3 | tubulin beta chain |
| 213 | Solanum lycoperscium | Solyc02g091870.3 | LOC101248155 | NC_015439.3 | tubulin alpha chain |
| 214 | Solanum lycoperscium | Solyc02g087880.3 | LOC101255154 | NC_015439.3 | tubulin alpha chain |
| 215 | Solanum lycoperscium | Solyc03g118760.3 | LOC101246411 | NC_015440.3 | tubulin beta chain |
| 216 | Solanum lycoperscium | Solyc12g089310.2 | LOC101248956 | NC_015449.3 | tubulin beta chain |
| 217 | Solanum tuberosum | PGSC0003DMG400001320 | LOC102587420 | NW_006238930.1 | alpha-tubulin |
| 218 | Solanum tuberosum | PGSC0003DMG400029337 | LOC102588315 | NW_006238962.1 | beta-tubulin |
| 219 | Solanum tuberosum | PGSC0003DMG400030627 | LOC102583337 | NW_006239181.1 | tubulin alpha-3 chain |
| 220 | Solanum tuberosum | PGSC0003DMG400015180 | LOC102582533 | NW_006239058.1 | gamma tubulin |
| 221 | Solanum tuberosum | PGSC0003DMG400011088 | LOC102585315 | NW_006238934.1 | tubulin beta chain |
| 222 | Solanum tuberosum | PGSC0003DMG400009938 | LOC102577624 | NW_006238958.1 | beta-tubulin |
| 223 | Solanum tuberosum | PGSC0003DMG400030431 | LOC102581203 | NW_006239053.1 | beta-tubulin 2 |
| 224 | Solanum tuberosum | PGSC0003DMG400020850 | LOC102594814 | NW_006239201.1 | beta-tubulin 16 |
| 225 | Solanum tuberosum | PGSC0003DMG400028193 | LOC102586422 | NW_006238934.1 | tubulin beta-1 chain |
| 226 | Solanum tuberosum | PGSC0003DMG400014296 | LOC102594131 | NW_006239079.1 | tubulin beta-1 chain |
| 227 | Zea mays | Zm00001eb218000 | LOC542417 | NC_050100.1 | beta tubulin 4 |
| 228 | Zea mays | Zm00001eb232910 | LOC100383576 | NC_050100.1 | tubulin beta-4 |
| 229 | Zea mays | Zm00001eb369310 | LOC100382290 | NC_050103.1 | beta tubulin6 |
| 230 | Zea mays | Zm00001eb000490 | LOC100273658 | NC_050096.1 | beta tubulin1 |
| 231 | Zea mays | Zm00001eb282650 | LOC542424 | NC_050101.1 | gamma tubulin |
| 232 | Zea mays | Zm00001eb215710 | LOC100381303 | NC_050100.1 | tubulin alpha-3 |
| 233 | Zea mays | Zm00001eb345620 | LOC542436 | NC_050103.1 | gamma-tubulin |
| 234 | Glycine max | GLYMA_10G251900 | LOC100799042 | NC_038256.2 | polyubiquitin |
| 235 | Glycine max | GLYMA_03G197600 | LOC100306626 | NC_016090.4 | uncharacterized |
| 236 | Glycine max | GLYMA_08G168200 | LOC100800163 | NC_038244.2 | ubiquitin-NEDD8 |
| 237 | Glycine max | GLYMA_07G199900 | LOC100817214 | NC_038243.2 | polyubiquitin |
| 238 | Oryza sativa | Os06g0650100 | LOC4341684 | NC_029261.1 | ubiquitin-NEDD8 |
| 239 | Oryza sativa | Os09g0420800 | LOC4347085 | NC_029264.1 | ubiquitin-NEDD8 |
| 240 | Oryza sativa | Os03g0808400 | LOC107276907 | NC_029258.1 | uncharacterized |
| 241 | Oryza sativa | Os10g0475900 | LOC4348886 | NC_029265.1 | polyubiquitin 12 |
| 242 | Oryza sativa | Os09g0452700 | LOC4347232 | NC_029264.1 | ubiquitin |
| 243 | Oryza sativa | Os04g0628100 | LOC4337080 | NC_029259.1 | polyubiquitin 3 |
| 244 | Sorghum bicolor | SORBI_3002G178800 | LOC8054608 | NC_012871.2 | ubiquitin |
| 245 | Sorghum bicolor | SORBI_3002G204200 | LOC8062113 | NC_012871.2 | ubiquitin-NEDD8 |
| 246 | Sorghum bicolor | SORBI_3002G308900 | LOC8080708 | NC_012871.2 | ubiquitin |
| 247 | Sorghum bicolor | SORBI_3002G309000 | LOC8077466 | NC_012871.2 | ubiquitin |
| 248 | Sorghum bicolor | SORBI_3002G292500 | LOC8077238 | NC_012871.2 | ubiquitin |
| 249 | Sorghum bicolor | SORBI_3010G210000 | LOC8069343 | NC_012879.2 | ubiquitin-NEDD8 |
| 250 | Sorghum bicolor | SORBI_3001G444800 | LOC8085240 | NC_012870.2 | ubiquitin-60S |
| 251 | Sorghum bicolor | SORBI_3004G049900 | LOC8076096 | NC_012873.2 | polyubiquitin |
| 252 | Solanum lycoperscium | Solyc07g064130.2 | LOC101258282 | NC_015444.3 | polyubiquitin |
| 253 | Solanum lycoperscium | Solyc11g005670.2 | LOC101267758 | NC_015448.3 | ubiquitin |
| 254 | Solanum lycoperscium | Solyc03g078630.3 | LOC101261102 | NC_015440.3 | polyubiquitin |
| 255 | Solanum lycoperscium | Solyc10g006480.2 | LOC101256039 | NC_015447.3 | polyubiquitin |
| 256 | Solanum lycoperscium | Solyc12g098940.2 | LOC101248559 | NC_015449.3 | ubiquitin-40S |
| 257 | Solanum tuberosum | PGSC0003DMG400011242 | LOC102587939 | NW_0062390720.1 | ubiquitin-NEDD8 |
| 258 | Solanum tuberosum | PGSC0003DMG400005862 | LOC102580286 | NW_0062396730.1 | ubiquitin-60S |
| 259 | Solanum tuberosum | PGSC0003DMG400003984 | LOC102587932 | NW_0062390540.1 | polyubiquitin- |
| 260 | Zea mays | Zm00001eb275020 | LOC103629697 | NC_050101.1 | ubiquitin-NEDD8 |
| 261 | Zea mays | Zm00001eb095960 | LOC103647262 | NC_050097.1 | ubiquitin-60S |
| 262 | Zea mays | Zm00001eb009920 | LOC103633261 | NC_050096.1 | ubiquitin-60S |
| 263 | Zea mays | Zm00001eb009900 | LOC103633247 | NC_050096.1 | ubiquitin-60S |

### Non-Coding Regions

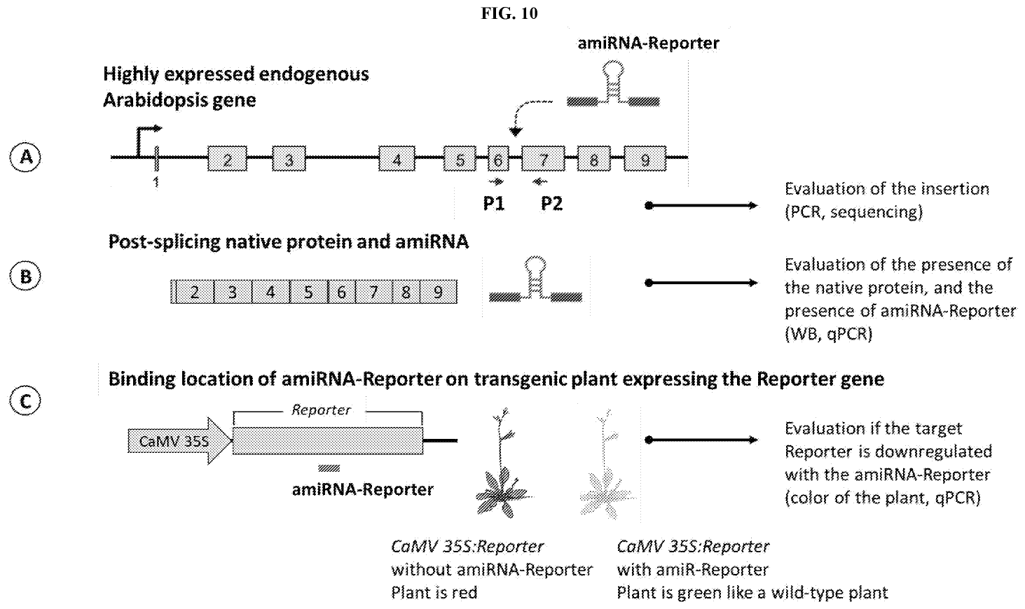

Provided herein, in certain embodiments, are cells comprising a non-coding region, wherein the non-coding region, such as an intron region or a 5′ non-coding region or a 3′ non-coding region, is modified (e.g., genetically edited) to comprise an endogenous or exogenous nucleic acid. As used herein, in some embodiments, a first modified non-coding or intron region refers to a non-coding, an intron, non-coding region, or intron region comprising an endogenous or exogenous nucleic acid. The endogenous or exogenous nucleic acid may be exogenous to the cell. The endogenous or exogenous nucleic acid may be exogenous to the non-coding region. The endogenous or exogenous nucleic acid may be endogenous to the cell. The endogenous or exogenous nucleic acid may be endogenous to the cell, and exogenous to the non-coding region. The endogenous or exogenous nucleic acid may be endogenous to the non-coding region. The first modified non-coding region may be present in any non-coding (e.g., intron) or non-coding (e.g., intron) region of a gene, e.g., the first modified intron region is present in the first, second, third, fourth, fifth, sixth, seventh, eighth, nineth, tenth, eleventh, twelfth, thirteenth, fourteenth, fifteenth, sixteenth, seventeenth, eighteenth, nineteenth, or twentieth intron of the gene, as applicable. For instance, in FIG. 10, the first modified intron region is present at intron 6 (between exon 6 and exon 7 of the gene). In some embodiments, the exogenous or endogenous nucleic acid is present in a 5′ non-coding region upstream of a gene. In some embodiments, the exogenous or endogenous nucleic acid is present in a 3′ non-coding region downstream of a gene. In some embodiments, the gene is selected from Table 1. In some embodiments, the intron is selected from Table 2. In some embodiments, the 5′ non-coding region or the 3′ non-coding region is a 5′ or 3′ non-coding region of a target gene from Table 1.

In some embodiments, the first modified non-coding region is modified from an intron of a gene. In some embodiments, the first modified non-coding region is modified from a 5′ non-coding region upstream of a gene. In some embodiments, the first modified non-coding region is modified from a 3′ noncoding region downstream of a gene. In some embodiments, the gene is endogenous to the cell. In some embodiments, the gene is selected from Table 1. In some embodiments, the gene comprises a plurality of introns. In some embodiments, the plurality of introns is about 2, 3, 4, 5, 6, 7, 8, 9, 10, 11, 12, 13, 14, 15, 16, 17, 18, 19, 20 introns (e.g., as exemplified by genes from Table 1). In some embodiments, the first modified intron region is present in the first, second, third, fourth, fifth, sixth, seventh, eighth, nineth, tenth, eleventh, twelfth, thirteenth, fourteenth, fifteenth, sixteenth, seventeenth, eighteenth, nineteenth, or twentieth intron of the gene, as applicable. In some embodiments, the gene is endogenous to the cell. In some embodiments, the gene is constitutively expressed. In some embodiments, the gene is expressed in a specific tissue or organ. In some embodiments, the cell is a plant cell, and the tissue or organ comprises a root, stem, fruit, seed, leaf, ground tissue, vascular tissue, or dermal tissue, or a combination of two or more thereof. In some embodiments, the gene is expressed at a range of 1-5%, 1-10%, 5-15%, or 5-20% of the total expressed genes in the cell (e.g., as determined by mRNA expression profiling of the said cell). In some embodiments, upon transcription and mRNA splicing, the native mRNA of the gene is translated into the native protein of the gene. In some embodiments, the gene encodes a native protein. A native protein may be a protein that has the same amino acid sequence as a protein endogenous to the cell. In some embodiments, the native protein is actin, ubiquitin, ribosomal protein, heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, RB7, or any other protein expressed from a gene of Table 1.

In some embodiments, the endogenous or exogenous nucleic acid is inserted in the non-coding region without nucleobase replacement. In other embodiments, the endogenous or exogenous nucleic acid is inserted in the non-coding region with replacement of one or more nucleobases of an endogenous non-coding region of the cell. In some cases, the endogenous or exogenous nucleic acid replaces at least 1, 5, 10, 15, 20, 25, 30, 35, 40, 45, 50 or more nucleobases of an endogenous non-coding region of the cell. In some cases, the exogenous nucleic acid replaces about 1-10, 1-15, 1-20, 1-25, 1-30, 1-35, 1-40, 1-45, 1-50, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, 45-50 nucleobases of an endogenous non-coding region of the cell. In example embodiments, editing non-coding region such as an intron, 5′ non-coding region or 3′ non-coding region, does not cause a mutation and/or frame shift to the native protein.

In some embodiments, a non-coding region is selected for modification based on the presence of an efficient and specific gRNA, an adequate distance from a splicing region, or the expression level of the non-coding region, or any combination of two or more thereof. In some embodiments, the non-coding region is an intron, 5′ non-coding region, or 3′ non-coding region.

In one aspect, the first non-coding region comprises a first portion of the endogenous non-coding region of the cell, the endogenous or exogenous nucleic acid, and a second portion of the endogenous non-coding region of the cell. In some embodiments, the non-coding region is an intron, 5′ non-coding region, or 3′ non-coding region. Non-limiting examples of the endogenous introns are described in Table 2.

TABLE 2
Examples of endogenous introns. The first column (SEQ ID NO)
contains the sequence identifier of non-limiting examples of
endogenous introns (SEQ ID NOS: 264-1274). The second column
(ENSEMBL IDENTIFIER) contains the code identifier of the gene
deposited in the EnsemblPlants database. The third column (INTRON
NUMBER) describes which intron on the gene of the second column
is, and its position between adjacent exons. A person of skill
in the art would be able to search the EnsemblPlants database
with the values of the second column and retrieve the information
of the intron and the corresponding FASTA sequence. The FASTA
sequence is available in the corresponding sequence listing
filed with the present application.

| SEQ | | |
|---|---|---|
| ID NO | ENSEMBL ID | INTRON |
| 264 | Os01g0269900 | Intron 1 |
| 265 | Os01g0282800 | Intron 1 |
| 266 | Os01g0964133 | Intron 1 |
| 267 | Os02g0167300 | Intron 1 |
| 268 | Os02g0167300 | Intron 2 |
| 269 | Os02g0596900 | Intron 1 |
| 270 | Os03g0105600 | Intron 1 |
| 271 | Os03g0219300 | Intron 1 |
| 272 | Os03g0219300 | Intron 2 |
| 273 | Os03g0219300 | Intron 3 |
| 274 | Os03g0219300 | Intron 4 |
| 275 | Os03g0661300 | Intron 1 |
| 276 | Os03g0661300 | Intron 2 |
| 277 | Os03g0718100 | Intron 1 |
| 278 | Os03g0718100 | Intron 2 |
| 279 | Os03g0718100 | Intron 3 |
| 280 | Os03g0726100 | Intron 1 |
| 281 | Os03g0780600 | Intron 1 |
| 282 | Os03g0783000 | Intron 1 |
| 283 | Os03g0783000 | Intron 2 |
| 284 | Os03g0783000 | Intron 3 |
| 285 | Os03g0783000 | Intron 4 |
| 286 | Os03g0783000 | Intron 5 |
| 287 | Os03g0783000 | Intron 6 |
| 288 | Os03g0783000 | Intron 7 |
| 289 | Os03g0808400 | Intron 1 |
| 290 | Os03g0808400 | Intron 2 |
| 291 | Os03g0836000 | Intron 1 |
| 292 | Os03g0836000 | Intron 2 |
| 293 | Os03g0836000 | Intron 3 |
| 294 | Os03g0836000 | Intron 4 |
| 295 | Os04g0177600 | Intron 10 |
| 296 | Os04g0177600 | Intron 11 |
| 297 | Os04g0177600 | Intron 12 |
| 298 | Os04g0177600 | Intron 13 |
| 299 | Os04g0177600 | Intron 14 |
| 300 | Os04g0177600 | Intron 15 |
| 301 | Os04g0177600 | Intron 16 |
| 302 | Os04g0177600 | Intron 17 |
| 303 | Os04g0177600 | Intron 18 |
| 304 | Os04g0177600 | Intron 19 |
| 305 | Os04g0177600 | Intron 1 |
| 306 | Os04g0177600 | Intron 20 |
| 307 | Os04g0177600 | Intron 2 |
| 308 | Os04g0177600 | Intron 3 |
| 309 | Os04g0177600 | Intron 4 |
| 310 | Os04g0177600 | Intron 5 |
| 311 | Os04g0177600 | Intron 6 |
| 312 | Os04g0177600 | Intron 7 |
| 313 | Os04g0177600 | Intron 8 |
| 314 | Os04g0177600 | Intron 9 |
| 315 | Os05g0106600 | Intron 1 |
| 316 | Os05g0413200 | Intron 1 |
| 317 | Os05g0413200 | Intron 2 |
| 318 | Os05g0413200 | Intron 3 |
| 319 | Os05g0438800 | Intron 1 |
| 320 | Os05g0438800 | Intron 2 |
| 321 | Os05g0438800 | Intron 3 |
| 322 | Os05g0438800 | Intron 4 |
| 323 | Os06g0650100 | Intron 1 |
| 324 | Os06g0671900 | Intron 1 |
| 325 | Os07g0574800 | Intron 1 |
| 326 | Os07g0574800 | Intron 2 |
| 327 | Os07g0574800 | Intron 3 |
| 328 | Os07g0574800 | Intron 4 |
| 329 | Os08g0137200 | Intron 1 |
| 330 | Os08g0369300 | Intron 3 |
| 331 | Os09g0452700 | Intron 1 |
| 332 | Os09g0452700 | Intron 2 |
| 333 | Os09g0452700 | Intron 3 |
| 334 | Os10g0475900 | Intron 1 |
| 335 | Os10g0510000 | Intron 1 |
| 336 | Os11g0163100 | Intron 1 |
| 337 | Os11g0163100 | Intron 2 |
| 338 | Os11g0163100 | Intron 3 |
| 339 | Os11g0163100 | Intron 4 |
| 340 | Os11g0247300 | Intron 1 |
| 341 | Os11g0247300 | Intron 2 |
| 342 | Os11g0247300 | Intron 3 |
| 343 | Os12g0163700 | Intron 1 |
| 344 | Os03t0718100 | Intron 1 |
| 345 | Os03t0718100 | Intron 2 |
| 346 | GLYMA_01G109300 | Intron 1 |
| 347 | GLYMA_01G109300 | Intron 2 |
| 348 | GLYMA_01G197500 | Intron 1 |
| 349 | GLYMA_01G197500 | Intron 2 |
| 350 | GLYMA_01G197500 | Intron 3 |
| 351 | GLYMA_02G091900 | Intron 1 |
| 352 | GLYMA_02G091900 | Intron 2 |
| 353 | GLYMA_02G091900 | Intron 3 |
| 354 | GLYMA_02G172800 | Intron 1 |
| 355 | GLYMA_02G172800 | Intron 2 |
| 356 | GLYMA_02G172800 | Intron 3 |
| 357 | GLYMA_02G248700 | Intron 1 |
| 358 | GLYMA_02G248700 | Intron 2 |
| 359 | GLYMA_02G248700 | Intron 3 |
| 360 | GLYMA_02G248700 | Intron 4 |
| 361 | GLYMA_02G248700 | Intron 5 |
| 362 | GLYMA_02G248700 | Intron 6 |
| 363 | GLYMA_02G248700 | Intron 7 |
| 364 | GLYMA_02G248700 | Intron 8 |
| 365 | GLYMA_02G248700 | Intron 9 |
| 366 | GLYMA_02G248700 | Intron 10 |
| 367 | GLYMA_02G248700 | Intron 11 |
| 368 | GLYMA_03G107300 | Intron 10 |
| 369 | GLYMA_03G107300 | Intron 11 |
| 370 | GLYMA_03G107300 | Intron 12 |
| 371 | GLYMA_03G107300 | Intron 13 |
| 372 | GLYMA_03G107300 | Intron 14 |
| 373 | GLYMA_03G107300 | Intron 15 |
| 374 | GLYMA_03G107300 | Intron 16 |
| 375 | GLYMA_03G107300 | Intron 17 |
| 376 | GLYMA_03G107300 | Intron 18 |
| 377 | GLYMA_03G107300 | Intron 19 |
| 378 | GLYMA_03G107300 | Intron 1 |
| 379 | GLYMA_03G107300 | Intron 2 |
| 380 | GLYMA_03G107300 | Intron 3 |
| 381 | GLYMA_03G107300 | Intron 4 |
| 382 | GLYMA_03G107300 | Intron 5 |
| 383 | GLYMA_03G107300 | Intron 6 |
| 384 | GLYMA_03G107300 | Intron 7 |
| 385 | GLYMA_03G107300 | Intron 8 |
| 386 | GLYMA_03G107300 | Intron 9 |
| 387 | GLYMA_03G124400 | Intron 1 |
| 388 | GLYMA_03G144800 | Intron 1 |
| 389 | GLYMA_03G144800 | Intron 2 |
| 390 | GLYMA_03G144800 | Intron 3 |
| 391 | GLYMA_03G197600 | Intron 1 |
| 392 | GLYMA_03G197600 | Intron 2 |
| 393 | GLYMA_03G197600 | Intron 3 |
| 394 | GLYMA_03G255800 | Intron 1 |
| 395 | GLYMA_03G255800 | Intron 2 |
| 396 | GLYMA_03G255800 | Intron 3 |
| 397 | GLYMA_03G255800 | Intron 4 |
| 398 | GLYMA_03G255800 | Intron 5 |
| 399 | GLYMA_03G255800 | Intron 6 |
| 400 | GLYMA_03G255800 | Intron 7 |
| 401 | GLYMA_03G255800 | Intron 8 |
| 402 | GLYMA_03G255800 | Intron 9 |
| 403 | GLYMA_03G255800 | Intron 10 |
| 404 | GLYMA_04G023900 | Intron 1 |
| 405 | GLYMA_04G023900 | Intron 2 |
| 406 | GLYMA_04G071500 | Intron 1 |
| 407 | GLYMA_04G071500 | Intron 2 |
| 408 | GLYMA_04G071500 | Intron 3 |
| 409 | GLYMA_04G071500 | Intron 4 |
| 410 | GLYMA_04G071500 | Intron 5 |
| 411 | GLYMA_04G071500 | Intron 6 |
| 412 | GLYMA_04G088500 | Intron 1 |
| 413 | GLYMA_04G088500 | Intron 2 |
| 414 | GLYMA_04G112600 | Intron 1 |
| 415 | GLYMA_04G112600 | Intron 2 |
| 416 | GLYMA_04G112600 | Intron 3 |
| 417 | GLYMA_04G112600 | Intron 4 |
| 418 | GLYMA_04G112600 | Intron 5 |
| 419 | GLYMA_04G112600 | Intron 6 |
| 420 | GLYMA_04G112600 | Intron 7 |
| 421 | GLYMA_04G112600 | Intron 8 |
| 422 | GLYMA_04G112600 | Intron 9 |
| 423 | GLYMA_04G112600 | Intron 10 |
| 424 | GLYMA_04G215900 | Intron 1 |
| 425 | GLYMA_04G215900 | Intron 2 |
| 426 | GLYMA_04G215900 | Intron 3 |
| 427 | GLYMA_05G000900 | Intron 1 |
| 428 | GLYMA_05G000900 | Intron 2 |
| 429 | GLYMA_05G000900 | Intron 3 |
| 430 | GLYMA_05G110200 | Intron 1 |
| 431 | GLYMA_05G110200 | Intron 2 |
| 432 | GLYMA_05G110200 | Intron 3 |
| 433 | GLYMA_05G126100 | Intron 1 |
| 434 | GLYMA_05G126100 | Intron 2 |
| 435 | GLYMA_05G172600 | Intron 1 |
| 436 | GLYMA_05G172600 | Intron 2 |
| 437 | GLYMA_05G172600 | Intron 3 |
| 438 | GLYMA_05G172600 | Intron 4 |
| 439 | GLYMA_05G172600 | Intron 5 |
| 440 | GLYMA_05G172600 | Intron 6 |
| 441 | GLYMA_05G172600 | Intron 7 |
| 442 | GLYMA_05G172600 | Intron 8 |
| 443 | GLYMA_05G207500 | Intron 1 |
| 444 | GLYMA_05G207500 | Intron 2 |
| 445 | GLYMA_06G090500 | Intron 1 |
| 446 | GLYMA_06G090500 | Intron 2 |
| 447 | GLYMA_07G118800 | Intron 1 |
| 448 | GLYMA_07G118800 | Intron 2 |
| 449 | GLYMA_07G118800 | Intron 3 |
| 450 | GLYMA_07G118800 | Intron 4 |
| 451 | GLYMA_07G118800 | Intron 5 |
| 452 | GLYMA_07G118800 | Intron 6 |
| 453 | GLYMA_07G118800 | Intron 7 |
| 454 | GLYMA_07G118800 | Intron 8 |
| 455 | GLYMA_07G118800 | Intron 9 |
| 456 | GLYMA_07G118800 | Intron 10 |
| 457 | GLYMA_07G118800 | Intron 11 |
| 458 | GLYMA_07G118800 | Intron 12 |
| 459 | GLYMA_07G118800 | Intron 13 |
| 460 | GLYMA_07G118800 | Intron 14 |
| 461 | GLYMA_07G118800 | Intron 15 |
| 462 | GLYMA_07G118800 | Intron 16 |
| 463 | GLYMA_07G118800 | Intron 17 |
| 464 | GLYMA_07G118800 | Intron 18 |
| 465 | GLYMA_07G118800 | Intron 19 |
| 466 | GLYMA_08G040300 | Intron 1 |
| 467 | GLYMA_08G040300 | Intron 2 |
| 468 | GLYMA_08G040300 | Intron 3 |
| 469 | GLYMA_08G040300 | Intron 4 |
| 470 | GLYMA_08G040300 | Intron 5 |
| 471 | GLYMA_08G040300 | Intron 6 |
| 472 | GLYMA_08G040300 | Intron 7 |
| 473 | GLYMA_08G040300 | Intron 8 |
| 474 | GLYMA_08G040300 | Intron 9 |
| 475 | GLYMA_08G040300 | Intron 10 |
| 476 | GLYMA_08G040300 | Intron 11 |
| 477 | GLYMA_08G040300 | Intron 12 |
| 478 | GLYMA_08G040300 | Intron 13 |
| 479 | GLYMA_08G040300 | Intron 14 |
| 480 | GLYMA_08G040300 | Intron 15 |
| 481 | GLYMA_08G081100 | Intron 1 |
| 482 | GLYMA_08G081100 | Intron 2 |
| 483 | GLYMA_08G168200 | Intron 1 |
| 484 | GLYMA_08G168200 | Intron 2 |
| 485 | GLYMA_08G182200 | Intron 1 |
| 486 | GLYMA_08G182200 | Intron 2 |
| 487 | GLYMA_08G182200 | Intron 3 |
| 488 | GLYMA_09G026100 | Intron 1 |
| 489 | GLYMA_09G026100 | Intron 2 |
| 490 | GLYMA_09G111200 | Intron 1 |
| 491 | GLYMA_09G111200 | Intron 2 |
| 492 | GLYMA_09G111200 | Intron 3 |
| 493 | GLYMA_09G229000 | Intron 1 |
| 494 | GLYMA_09G229000 | Intron 2 |
| 495 | GLYMA_09G229000 | Intron 3 |
| 496 | GLYMA_09G229000 | Intron 4 |
| 497 | GLYMA_09G229000 | Intron 5 |
| 498 | GLYMA_09G229000 | Intron 6 |
| 499 | GLYMA_10G089200 | Intron 1 |
| 500 | GLYMA_10G089200 | Intron 2 |
| 501 | GLYMA_10G089200 | Intron 3 |
| 502 | GLYMA_10G089200 | Intron 4 |
| 503 | GLYMA_10G089200 | Intron 5 |
| 504 | GLYMA_10G089200 | Intron 6 |
| 505 | GLYMA_10G188100 | Intron 1 |
| 506 | GLYMA_10G188100 | Intron 2 |
| 507 | GLYMA_10G188100 | Intron 3 |
| 508 | GLYMA_10G188100 | Intron 4 |
| 509 | GLYMA_10G188100 | Intron 5 |
| 510 | GLYMA_10G188100 | Intron 6 |
| 511 | GLYMA_10G188100 | Intron 7 |
| 512 | GLYMA_10G188100 | Intron 8 |
| 513 | GLYMA_10G188100 | Intron 9 |
| 514 | GLYMA_10G188100 | Intron 10 |
| 515 | GLYMA_10G188100 | Intron 11 |
| 516 | GLYMA_10G188100 | Intron 12 |
| 517 | GLYMA_10G188100 | Intron 13 |
| 518 | GLYMA_10G188100 | Intron 14 |
| 519 | GLYMA_10G188100 | Intron 15 |
| 520 | GLYMA_10G188100 | Intron 16 |
| 521 | GLYMA_10G188100 | Intron 17 |
| 522 | GLYMA_10G188100 | Intron 18 |
| 523 | GLYMA_10G188100 | Intron 19 |
| 524 | GLYMA_10G235100 | Intron 1 |
| 525 | GLYMA_10G235100 | Intron 2 |
| 526 | GLYMA_10G255500 | Intron 1 |
| 527 | GLYMA_10G255500 | Intron 2 |
| 528 | GLYMA_10G255500 | Intron 3 |
| 529 | GLYMA_11G044200 | Intron 1 |
| 530 | GLYMA_11G044200 | Intron 2 |
| 531 | GLYMA_11G044200 | Intron 3 |
| 532 | GLYMA_11G139800 | Intron 1 |
| 533 | GLYMA_11G139800 | Intron 2 |
| 534 | GLYMA_11G139800 | Intron 3 |
| 535 | GLYMA_11G219700 | Intron 1 |
| 536 | GLYMA_11G219700 | Intron 2 |
| 537 | GLYMA_11G219700 | Intron 3 |
| 538 | GLYMA_11G219700 | Intron 4 |
| 539 | GLYMA_11G219700 | Intron 5 |
| 540 | GLYMA_11G219700 | Intron 6 |
| 541 | GLYMA_11G219700 | Intron 7 |
| 542 | GLYMA_11G219700 | Intron 8 |
| 543 | GLYMA_11G219700 | Intron 9 |
| 544 | GLYMA_11G219700 | Intron 10 |
| 545 | GLYMA_11G219700 | Intron 11 |
| 546 | GLYMA_12G007600 | Intron 1 |
| 547 | GLYMA_12G007600 | Intron 2 |
| 548 | GLYMA_12G007600 | Intron 3 |
| 549 | GLYMA_12G007600 | Intron 4 |
| 550 | GLYMA_12G007600 | Intron 5 |
| 551 | GLYMA_12G007600 | Intron 6 |
| 552 | GLYMA_12G063400 | Intron 1 |
| 553 | GLYMA_12G063400 | Intron 2 |
| 554 | GLYMA_12G063400 | Intron 3 |
| 555 | GLYMA_13G335600 | Intron 1 |
| 556 | GLYMA_13G335600 | Intron 2 |
| 557 | GLYMA_13G335600 | Intron 3 |
| 558 | GLYMA_14G067800 | Intron 1 |
| 559 | GLYMA_14G067800 | Intron 2 |
| 560 | GLYMA_14G067800 | Intron 3 |
| 561 | GLYMA_14G067800 | Intron 4 |
| 562 | GLYMA_14G067800 | Intron 5 |
| 563 | GLYMA_14G067800 | Intron 6 |
| 564 | GLYMA_14G067800 | Intron 7 |
| 565 | GLYMA_14G067800 | Intron 8 |
| 566 | GLYMA_15G038700 | Intron 1 |
| 567 | GLYMA_15G038700 | Intron 2 |
| 568 | GLYMA_15G038700 | Intron 3 |
| 569 | GLYMA_15G050200 | Intron 1 |
| 570 | GLYMA_15G050200 | Intron 2 |
| 571 | GLYMA_15G050200 | Intron 3 |
| 572 | GLYMA_16G053400 | Intron 1 |
| 573 | GLYMA_16G053400 | Intron 2 |
| 574 | GLYMA_16G053400 | Intron 3 |
| 575 | GLYMA_16G053400 | Intron 4 |
| 576 | GLYMA_16G053400 | Intron 5 |
| 577 | GLYMA_16G053400 | Intron 6 |
| 578 | GLYMA_16G053400 | Intron 7 |
| 579 | GLYMA_16G053400 | Intron 8 |
| 580 | GLYMA_16G053400 | Intron 9 |
| 581 | GLYMA_16G053400 | Intron 10 |
| 582 | GLYMA_16G053400 | Intron 11 |
| 583 | GLYMA_16G053400 | Intron 12 |
| 584 | GLYMA_16G053400 | Intron 13 |
| 585 | GLYMA_16G053400 | Intron 14 |
| 586 | GLYMA_16G096700 | Intron 1 |
| 587 | GLYMA_16G096700 | Intron 2 |
| 588 | GLYMA_16G096700 | Intron 3 |
| 589 | GLYMA_16G096700 | Intron 4 |
| 590 | GLYMA_16G096700 | Intron 5 |
| 591 | GLYMA_16G096700 | Intron 6 |
| 592 | GLYMA_16G096700 | Intron 7 |
| 593 | GLYMA_16G096700 | Intron 8 |
| 594 | GLYMA_16G096700 | Intron 9 |
| 595 | GLYMA_16G096700 | Intron 10 |
| 596 | GLYMA_16G096700 | Intron 11 |
| 597 | GLYMA_16G154000 | Intron 1 |
| 598 | GLYMA_16G154000 | Intron 2 |
| 599 | GLYMA_16G154000 | Intron 3 |
| 600 | GLYMA_16G154000 | Intron 4 |
| 601 | GLYMA_17G258300 | Intron 1 |
| 602 | GLYMA_17G258300 | Intron 2 |
| 603 | GLYMA_18G037700 | Intron 1 |
| 604 | GLYMA_18G037700 | Intron 2 |
| 605 | GLYMA_18G037700 | Intron 3 |
| 606 | GLYMA_18G037700 | Intron 4 |
| 607 | GLYMA_18G037700 | Intron 5 |
| 608 | GLYMA_18G037700 | Intron 6 |
| 609 | GLYMA_18G037700 | Intron 7 |
| 610 | GLYMA_18G037700 | Intron 8 |
| 611 | GLYMA_18G037700 | Intron 9 |
| 612 | GLYMA_18G037700 | Intron 10 |
| 613 | GLYMA_18G037700 | Intron 11 |
| 614 | GLYMA_18G290800 | Intron 1 |
| 615 | GLYMA_18G290800 | Intron 2 |
| 616 | GLYMA_18G290800 | Intron 3 |
| 617 | GLYMA_19G000900 | Intron 1 |
| 618 | GLYMA_19G000900 | Intron 2 |
| 619 | GLYMA_19G000900 | Intron 3 |
| 620 | GLYMA_19G095900 | Intron 1 |
| 621 | GLYMA_19G095900 | Intron 2 |
| 622 | GLYMA_19G095900 | Intron 3 |
| 623 | GLYMA_19G095900 | Intron 4 |
| 624 | GLYMA_19G095900 | Intron 5 |
| 625 | GLYMA_19G095900 | Intron 6 |
| 626 | GLYMA_19G095900 | Intron 7 |
| 627 | GLYMA_19G095900 | Intron 8 |
| 628 | GLYMA_19G095900 | Intron 9 |
| 629 | GLYMA_19G095900 | Intron 10 |
| 630 | GLYMA_19G095900 | Intron 11 |
| 631 | GLYMA_19G095900 | Intron 12 |
| 632 | GLYMA_19G095900 | Intron 13 |
| 633 | GLYMA_19G095900 | Intron 14 |
| 634 | GLYMA_19G113000 | Intron 1 |
| 635 | GLYMA_19G113000 | Intron 2 |
| 636 | GLYMA_19G113000 | Intron 3 |
| 637 | GLYMA_19G113000 | Intron 4 |
| 638 | GLYMA_19G127700 | Intron 1 |
| 639 | GLYMA_19G147900 | Intron 1 |
| 640 | GLYMA_19G147900 | Intron 2 |
| 641 | GLYMA_19G147900 | Intron 3 |
| 642 | GLYMA_19G253300 | Intron 1 |
| 643 | GLYMA_19G253300 | Intron 2 |
| 644 | GLYMA_19G253300 | Intron 3 |
| 645 | GLYMA_19G253300 | Intron 4 |
| 646 | GLYMA_19G253300 | Intron 5 |
| 647 | GLYMA_19G253300 | Intron 6 |
| 648 | GLYMA_19G253300 | Intron 7 |
| 649 | GLYMA_19G253300 | Intron 8 |
| 650 | GLYMA_19G253300 | Intron 9 |
| 651 | GLYMA_19G253300 | Intron 10 |
| 652 | GLYMA_20G136000 | Intron 1 |
| 653 | GLYMA_20G136000 | Intron 2 |
| 654 | GLYMA_20G136000 | Intron 3 |
| 655 | GLYMA_20G159200 | Intron 1 |
| 656 | GLYMA_20G159200 | Intron 2 |
| 657 | GLYMA_20G202700 | Intron 1 |
| 658 | GLYMA_20G202700 | Intron 2 |
| 659 | GLYMA_20G202700 | Intron 3 |
| 660 | GLYMA_20G202700 | Intron 4 |
| 661 | GLYMA_20G202700 | Intron 5 |
| 662 | GLYMA_20G202700 | Intron 6 |
| 663 | GLYMA_20G202700 | Intron 7 |
| 664 | GLYMA_20G202700 | Intron 8 |
| 665 | GLYMA_20G202700 | Intron 9 |
| 666 | GLYMA_20G202700 | Intron 10 |
| 667 | GLYMA_20G202700 | Intron 11 |
| 668 | GLYMA_20G202700 | Intron 12 |
| 669 | GLYMA_20G202700 | Intron 13 |
| 670 | GLYMA_20G202700 | Intron 14 |
| 671 | GLYMA_20G202700 | Intron 15 |
| 672 | GLYMA_20G202700 | Intron 16 |
| 673 | GLYMA_20G202700 | Intron 17 |
| 674 | GLYMA_20G202700 | Intron 18 |
| 675 | GLYMA_20G202700 | Intron 19 |
| 676 | GLYMA_02G091900 | Intron 2 |
| 677 | Solyc00g017210.2 | Intron 1 |
| 678 | Solyc00g017210.2 | Intron 2 |
| 679 | Solyc00g017210.2 | Intron 3 |
| 680 | Solyc02g087880.3 | Intron 1 |
| 681 | Solyc02g087880.3 | Intron 2 |
| 682 | Solyc02g087880.3 | Intron 3 |
| 683 | Solyc02g091870.3 | Intron 1 |
| 684 | Solyc02g091870.3 | Intron 2 |
| 685 | Solyc02g091870.3 | Intron 3 |
| 686 | Solyc03g025730.3 | Intron 1 |
| 687 | Solyc03g025730.3 | Intron 2 |
| 688 | Solyc03g078400.3 | Intron 1 |
| 689 | Solyc03g078400.3 | Intron 2 |
| 690 | Solyc03g078400.3 | Intron 3 |
| 691 | Solyc03g078400.3 | Intron 4 |
| 692 | Solyc03g078630.3 | Intron 1 |
| 693 | Solyc03g111380.3 | Intron 1 |
| 694 | Solyc03g111380.3 | Intron 2 |
| 695 | Solyc03g111380.3 | Intron 3 |
| 696 | Solyc03g111380.3 | Intron 4 |
| 697 | Solyc03g111380.3 | Intron 5 |
| 698 | Solyc03g111380.3 | Intron 6 |
| 699 | Solyc03g111380.3 | Intron 7 |
| 700 | Solyc03g111380.3 | Intron 8 |
| 701 | Solyc03g111380.3 | Intron 9 |
| 702 | Solyc03g111380.3 | Intron 10 |
| 703 | Solyc03g118760.3 | Intron 1 |
| 704 | Solyc03g118760.3 | Intron 2 |
| 705 | Solyc04g011500.3 | Intron 1 |
| 706 | Solyc04g011500.3 | Intron 2 |
| 707 | Solyc04g011500.3 | Intron 3 |
| 708 | Solyc04g011500.3 | Intron 4 |
| 709 | Solyc04g024530.3 | Intron 1 |
| 710 | Solyc04g024530.3 | Intron 2 |
| 711 | Solyc04g024530.3 | Intron 3 |
| 712 | Solyc04g024530.3 | Intron 4 |
| 713 | Solyc04g024530.3 | Intron 5 |
| 714 | Solyc04g024530.3 | Intron 6 |
| 715 | Solyc04g071260.3 | Intron 1 |
| 716 | Solyc04g071260.3 | Intron 2 |
| 717 | Solyc04g071260.3 | Intron 3 |
| 718 | Solyc04g077020.3 | Intron 1 |
| 719 | Solyc04g077020.3 | Intron 2 |
| 720 | Solyc04g077020.3 | Intron 3 |
| 721 | Solyc04g077020.3 | Intron 4 |
| 722 | Solyc04g081490.3 | Intron 1 |
| 723 | Solyc04g081490.3 | Intron 2 |
| 724 | Solyc05g013940.3 | Intron 1 |
| 725 | Solyc05g013940.3 | Intron 2 |
| 726 | Solyc05g013940.3 | Intron 3 |
| 727 | Solyc05g013940.3 | Intron 4 |
| 728 | Solyc05g013940.3 | Intron 5 |
| 729 | Solyc05g013940.3 | Intron 6 |
| 730 | Solyc05g013940.3 | Intron 7 |
| 731 | Solyc05g013940.3 | Intron 8 |
| 732 | Solyc05g018600.3 | Intron 1 |
| 733 | Solyc05g018600.3 | Intron 2 |
| 734 | Solyc05g018600.3 | Intron 3 |
| 735 | Solyc05g018600.3 | Intron 4 |
| 736 | Solyc05g018600.3 | Intron 5 |
| 737 | Solyc05g018600.3 | Intron 6 |
| 738 | Solyc06g043175.1 | Intron 1 |
| 739 | Solyc06g043175.1 | Intron 2 |
| 740 | Solyc06g043175.1 | Intron 3 |
| 741 | Solyc06g043175.1 | Intron 4 |
| 742 | Solyc06g043175.1 | Intron 5 |
| 743 | Solyc06g043175.1 | Intron 6 |
| 744 | Solyc06g043175.1 | Intron 7 |
| 745 | Solyc06g043175.1 | Intron 8 |
| 746 | Solyc06g043175.1 | Intron 9 |
| 747 | Solyc06g043175.1 | Intron 10 |
| 748 | Solyc06g043175.1 | Intron 11 |
| 749 | Solyc06g043175.1 | Intron 12 |
| 750 | Solyc06g043175.1 | Intron 13 |
| 751 | Solyc06g076090.3 | Intron 1 |
| 752 | Solyc06g076090.3 | Intron 2 |
| 753 | Solyc06g076090.3 | Intron 3 |
| 754 | Solyc06g076090.3 | Intron 4 |
| 755 | Solyc07g064130.2 | Intron 1 |
| 756 | Solyc07g066120.3 | Intron 1 |
| 757 | Solyc07g066120.3 | Intron 2 |
| 758 | Solyc07g066120.3 | Intron 3 |
| 759 | Solyc07g066120.3 | Intron 4 |
| 760 | Solyc07g066120.3 | Intron 5 |
| 761 | Solyc07g066120.3 | Intron 6 |
| 762 | Solyc07g066120.3 | Intron 7 |
| 763 | Solyc07g066120.3 | Intron 8 |
| 764 | Solyc07g066120.3 | Intron 9 |
| 765 | Solyc07g066120.3 | Intron 10 |
| 766 | Solyc07g066120.3 | Intron 11 |
| 767 | Solyc08g006890.3 | Intron 1 |
| 768 | Solyc08g006890.3 | Intron 2 |
| 769 | Solyc08g006890.3 | Intron 3 |
| 770 | Solyc09g089660.3 | Intron 1 |
| 771 | Solyc09g089660.3 | Intron 2 |
| 772 | Solyc09g089660.3 | Intron 3 |
| 773 | Solyc09g089660.3 | Intron 4 |
| 774 | Solyc09g089660.3 | Intron 5 |
| 775 | Solyc09g089660.3 | Intron 6 |
| 776 | Solyc09g089660.3 | Intron 7 |
| 777 | Solyc09g089660.3 | Intron 8 |
| 778 | Solyc09g089660.3 | Intron 9 |
| 779 | Solyc09g089660.3 | Intron 10 |
| 780 | Solyc09g089660.3 | Intron 11 |
| 781 | Solyc09g089660.3 | Intron 12 |
| 782 | Solyc09g089660.3 | Intron 13 |
| 783 | Solyc09g089660.3 | Intron 14 |
| 784 | Solyc09g089660.3 | Intron 15 |
| 785 | Solyc09g089660.3 | Intron 16 |
| 786 | Solyc09g089660.3 | Intron 17 |
| 787 | Solyc09g089660.3 | Intron 18 |
| 788 | Solyc09g089660.3 | Intron 19 |
| 789 | Solyc09g089660.3 | Intron 20 |
| 790 | Solyc10g006480.2 | Intron 1 |
| 791 | Solyc10g080500.2 | Intron 1 |
| 792 | Solyc10g080500.2 | Intron 2 |
| 793 | Solyc10g080500.2 | Intron 3 |
| 794 | Solyc10g080500.2 | Intron 4 |
| 795 | Solyc10g086460.2 | Intron 1 |
| 796 | Solyc10g086460.2 | Intron 2 |
| 797 | Solyc10g086460.2 | Intron 3 |
| 798 | Solyc10g086760.2 | Intron 1 |
| 799 | Solyc10g086760.2 | Intron 2 |
| 800 | Solyc11g005330.2 | Intron 1 |
| 801 | Solyc11g005330.2 | Intron 2 |
| 802 | Solyc11g005330.2 | Intron 3 |
| 803 | Solyc11g005330.2 | Intron 4 |
| 804 | Solyc11g005330.2 | Intron 5 |
| 805 | Solyc11g005670.2 | Intron 1 |
| 806 | Solyc11g005670.2 | Intron 2 |
| 807 | Solyc11g065990.2 | Intron 1 |
| 808 | Solyc11g065990.2 | Intron 2 |
| 809 | Solyc11g065990.2 | Intron 3 |
| 810 | Solyc12g037980.2 | Intron 1 |
| 811 | Solyc12g037980.2 | Intron 2 |
| 812 | Solyc12g037980.2 | Intron 3 |
| 813 | Solyc12g037980.2 | Intron 4 |
| 814 | Solyc12g037980.2 | Intron 5 |
| 815 | Solyc12g037980.2 | Intron 6 |
| 816 | Solyc12g037980.2 | Intron 7 |
| 817 | Solyc12g037980.2 | Intron 8 |
| 818 | Solyc12g037980.2 | Intron 9 |
| 819 | Solyc12g037980.2 | Intron 10 |
| 820 | Solyc12g037980.2 | Intron 11 |
| 821 | Solyc12g037980.2 | Intron 12 |
| 822 | Solyc12g037980.2 | Intron 13 |
| 823 | Solyc12g037980.2 | Intron 14 |
| 824 | Solyc12g037980.2 | Intron 15 |
| 825 | Solyc12g037980.2 | Intron 16 |
| 826 | Solyc12g037980.2 | Intron 17 |
| 827 | Solyc12g037980.2 | Intron 18 |
| 828 | Solyc12g037980.2 | Intron 19 |
| 829 | Solyc12g089310.2 | Intron 1 |
| 830 | Solyc12g089310.2 | Intron 2 |
| 831 | SORBI_3001G022800 | Intron 1 |
| 832 | SORBI_3001G022800 | Intron 2 |
| 833 | SORBI_3001G022800 | Intron 3 |
| 834 | SORBI_3001G069800 | Intron 1 |
| 835 | SORBI_3001G069800 | Intron 2 |
| 836 | SORBI_3001G073700 | Intron 1 |
| 837 | SORBI_3001G073700 | Intron 2 |
| 838 | SORBI_3001G073700 | Intron 3 |
| 839 | SORBI_3001G146000 | Intron 1 |
| 840 | SORBI_3001G146000 | Intron 2 |
| 841 | SORBI_3001G197400 | Intron 1 |
| 842 | SORBI_3001G197400 | Intron 2 |
| 843 | SORBI_3001G197400 | Intron 3 |
| 844 | SORBI_3001G234200 | Intron 1 |
| 845 | SORBI_3001G234200 | Intron 2 |
| 846 | SORBI_3001G234200 | Intron 3 |
| 847 | SORBI_3001G234200 | Intron 4 |
| 848 | SORBI_3001G234200 | Intron 5 |
| 849 | SORBI_3001G234200 | Intron 6 |
| 850 | SORBI_3001G444800 | Intron 1 |
| 851 | SORBI_3001G444800 | Intron 2 |
| 852 | SORBI_3001G444800 | Intron 3 |
| 853 | SORBI_3001G453700 | Intron 1 |
| 854 | SORBI_3001G453700 | Intron 2 |
| 855 | SORBI_3001G453700 | Intron 3 |
| 856 | SORBI_3001G453700 | Intron 4 |
| 857 | SORBI_3001G536000 | Intron 1 |
| 858 | SORBI_3001G536000 | Intron 2 |
| 859 | SORBI_3001G536000 | Intron 3 |
| 860 | SORBI_3001G536000 | Intron 4 |
| 861 | SORBI_3001G536000 | Intron 5 |
| 862 | SORBI_3001G536000 | Intron 6 |
| 863 | SORBI_3001G536000 | Intron 7 |
| 864 | SORBI_3001G536000 | Intron 8 |
| 865 | SORBI_3001G536000 | Intron 9 |
| 866 | SORBI_3001G536000 | Intron 10 |
| 867 | SORBI_3001G536000 | Intron 11 |
| 868 | SORBI_3001G536000 | Intron 12 |
| 869 | SORBI_3001G536000 | Intron 13 |
| 870 | SORBI_3001G536000 | Intron 14 |
| 871 | SORBI_3001G536000 | Intron 15 |
| 872 | SORBI_3001G536000 | Intron 16 |
| 873 | SORBI_3001G536000 | Intron 17 |
| 874 | SORBI_3001G536000 | Intron 18 |
| 875 | SORBI_3001G536000 | Intron 19 |
| 876 | SORBI_3001G540900 | Intron 1 |
| 877 | SORBI_3002G178800 | Intron 1 |
| 878 | SORBI_3002G204200 | Intron 1 |
| 879 | SORBI_3002G204200 | Intron 2 |
| 880 | SORBI_3002G292500 | Intron 1 |
| 881 | SORBI_3002G292500 | Intron 2 |
| 882 | SORBI_3002G308900 | Intron 1 |
| 883 | SORBI_3002G308900 | Intron 2 |
| 884 | SORBI_3002G308900 | Intron 3 |
| 885 | SORBI_3002G309000 | Intron 1 |
| 886 | SORBI_3002G309000 | Intron 2 |
| 887 | SORBI_3002G309000 | Intron 3 |
| 888 | SORBI_3002G426300 | Intron 1 |
| 889 | SORBI_3002G426300 | Intron 2 |
| 890 | SORBI_3002G426300 | Intron 3 |
| 891 | SORBI_3002G426300 | Intron 4 |
| 892 | SORBI_3002G426300 | Intron 5 |
| 893 | SORBI_3002G426300 | Intron 6 |
| 894 | SORBI_3002G426300 | Intron 7 |
| 895 | SORBI_3002G426300 | Intron 8 |
| 896 | SORBI_3002G426300 | Intron 9 |
| 897 | SORBI_3002G426300 | Intron 10 |
| 898 | SORBI_3002G426300 | Intron 11 |
| 899 | SORBI_3002G426300 | Intron 12 |
| 900 | SORBI_3002G426300 | Intron 13 |
| 901 | SORBI_3002G426300 | Intron 14 |
| 902 | SORBI_3003G074200 | Intron 1 |
| 903 | SORBI_3003G074200 | Intron 2 |
| 904 | SORBI_3003G074200 | Intron 3 |
| 905 | SORBI_3003G074200 | Intron 4 |
| 906 | SORBI_3003G074200 | Intron 5 |
| 907 | SORBI_3003G074200 | Intron 6 |
| 908 | SORBI_3003G074200 | Intron 7 |
| 909 | SORBI_3003G074200 | Intron 8 |
| 910 | SORBI_3003G074200 | Intron 9 |
| 911 | SORBI_3003G074200 | Intron 10 |
| 912 | SORBI_3003G074200 | Intron 11 |
| 913 | SORBI_3003G328800 | Intron 1 |
| 914 | SORBI_3003G328800 | Intron 2 |
| 915 | SORBI_3003G367300 | Intron 1 |
| 916 | SORBI_3003G367300 | Intron 2 |
| 917 | SORBI_3003G367300 | Intron 3 |
| 918 | SORBI_3004G053300 | Intron 1 |
| 919 | SORBI_3004G053300 | Intron 2 |
| 920 | SORBI_3004G203500 | Intron 1 |
| 921 | SORBI_3004G203500 | Intron 2 |
| 922 | SORBI_3004G203500 | Intron 3 |
| 923 | SORBI_3004G203500 | Intron 4 |
| 924 | SORBI_3004G203500 | Intron 5 |
| 925 | SORBI_3004G203500 | Intron 6 |
| 926 | SORBI_3004G203500 | Intron 7 |
| 927 | SORBI_3004G203500 | Intron 8 |
| 928 | SORBI_3004G203500 | Intron 9 |
| 929 | SORBI_3005G047100 | Intron 1 |
| 930 | SORBI_3005G047100 | Intron 2 |
| 931 | SORBI_3005G047100 | Intron 3 |
| 932 | SORBI_3006G029100 | Intron 1 |
| 933 | SORBI_3006G029100 | Intron 2 |
| 934 | SORBI_3006G029100 | Intron 3 |
| 935 | SORBI_3006G029100 | Intron 4 |
| 936 | SORBI_3006G029100 | Intron 5 |
| 937 | SORBI_3006G029100 | Intron 6 |
| 938 | SORBI_3006G029100 | Intron 7 |
| 939 | SORBI_3006G029100 | Intron 8 |
| 940 | SORBI_3006G029100 | Intron 9 |
| 941 | SORBI_3006G029100 | Intron 10 |
| 942 | SORBI_3006G029100 | Intron 11 |
| 943 | SORBI_3006G029100 | Intron 12 |
| 944 | SORBI_3006G029100 | Intron 13 |
| 945 | SORBI_3006G029100 | Intron 14 |
| 946 | SORBI_3006G029100 | Intron 15 |
| 947 | SORBI_3006G029100 | Intron 16 |
| 948 | SORBI_3006G029100 | Intron 17 |
| 949 | SORBI_3006G029100 | Intron 18 |
| 950 | SORBI_3006G029100 | Intron 19 |
| 951 | SORBI_3006G257800 | Intron 1 |
| 952 | SORBI_3006G257800 | Intron 2 |
| 953 | SORBI_3006G257800 | Intron 3 |
| 954 | SORBI_3006G257800 | Intron 4 |
| 955 | SORBI_3006G257800 | Intron 5 |
| 956 | SORBI_3006G257800 | Intron 6 |
| 957 | SORBI_3006G257800 | Intron 7 |
| 958 | SORBI_3006G257800 | Intron 8 |
| 959 | SORBI_3006G257800 | Intron 9 |
| 960 | SORBI_3006G257800 | Intron 10 |
| 961 | SORBI_3006G257800 | Intron 11 |
| 962 | SORBI_3007G026800 | Intron 1 |
| 963 | SORBI_3007G026800 | Intron 2 |
| 964 | SORBI_3007G026800 | Intron 3 |
| 965 | SORBI_3007G026800 | Intron 4 |
| 966 | SORBI_3007G026800 | Intron 5 |
| 967 | SORBI_3007G026800 | Intron 6 |
| 968 | SORBI_3007G026800 | Intron 7 |
| 969 | SORBI_3007G026800 | Intron 8 |
| 970 | SORBI_3007G026800 | Intron 9 |
| 971 | SORBI_3008G047000 | Intron 1 |
| 972 | SORBI_3008G047000 | Intron 2 |
| 973 | SORBI_3008G047000 | Intron 3 |
| 974 | SORBI_3008G173100 | Intron 1 |
| 975 | SORBI_3008G173100 | Intron 2 |
| 976 | SORBI_3008G173100 | Intron 3 |
| 977 | SORBI_3008G173100 | Intron 4 |
| 978 | SORBI_3008G173100 | Intron 5 |
| 979 | SORBI_3008G173100 | Intron 6 |
| 980 | SORBI_3009G005900 | Intron 1 |
| 981 | SORBI_3009G005900 | Intron 2 |
| 982 | SORBI_3009G005900 | Intron 3 |
| 983 | SORBI_3009G052100 | Intron 1 |
| 984 | SORBI_3009G052100 | Intron 2 |
| 985 | SORBI_3009G052100 | Intron 3 |
| 986 | SORBI_3009G052100 | Intron 4 |
| 987 | SORBI_3009G052100 | Intron 5 |
| 988 | SORBI_3009G052100 | Intron 6 |
| 989 | SORBI_3009G052100 | Intron 7 |
| 990 | SORBI_3009G052100 | Intron 8 |
| 991 | SORBI_3009G052100 | Intron 9 |
| 992 | SORBI_3009G052100 | Intron 10 |
| 993 | SORBI_3009G153000 | Intron 1 |
| 994 | SORBI_3009G153000 | Intron 2 |
| 995 | SORBI_3009G153000 | Intron 3 |
| 996 | SORBI_3010G210000 | Intron 1 |
| 997 | SORBI_3010G210000 | Intron 2 |
| 998 | SORBI_3010G224900 | Intron 1 |
| 999 | SORBI_3010G224900 | Intron 2 |
| 1000 | SORBI_3005G047100 | Intron 2 |
| 1001 | SORBI_3005G047100 | Intron 3 |
| 1002 | PGSC0003DMG400001320 | Intron 1 |
| 1003 | PGSC0003DMG400001320 | Intron 2 |
| 1004 | PGSC0003DMG400001320 | Intron 3 |
| 1005 | PGSC0003DMG400005862 | Intron 1 |
| 1006 | PGSC0003DMG400005862 | Intron 2 |
| 1007 | PGSC0003DMG400005862 | Intron 3 |
| 1008 | PGSC0003DMG400008618 | Intron 1 |
| 1009 | PGSC0003DMG400008618 | Intron 2 |
| 1010 | PGSC0003DMG400008619 | Intron 1 |
| 1011 | PGSC0003DMG400008619 | Intron 2 |
| 1012 | PGSC0003DMG400008912 | Intron 1 |
| 1013 | PGSC0003DMG400008912 | Intron 2 |
| 1014 | PGSC0003DMG400008912 | Intron 3 |
| 1015 | PGSC0003DMG400008912 | Intron 4 |
| 1016 | PGSC0003DMG400009938 | Intron 1 |
| 1017 | PGSC0003DMG400009938 | Intron 2 |
| 1018 | PGSC0003DMG400010772 | Intron 1 |
| 1019 | PGSC0003DMG400010772 | Intron 2 |
| 1020 | PGSC0003DMG400010772 | Intron 3 |
| 1021 | PGSC0003DMG400010772 | Intron 4 |
| 1022 | PGSC0003DMG400010772 | Intron 5 |
| 1023 | PGSC0003DMG400010772 | Intron 6 |
| 1024 | PGSC0003DMG400010772 | Intron 7 |
| 1025 | PGSC0003DMG400010772 | Intron 8 |
| 1026 | PGSC0003DMG400011088 | Intron 1 |
| 1027 | PGSC0003DMG400011088 | Intron 2 |
| 1028 | PGSC0003DMG400011242 | Intron 1 |
| 1029 | PGSC0003DMG400011242 | Intron 2 |
| 1030 | PGSC0003DMG400014296 | Intron 1 |
| 1031 | PGSC0003DMG400014296 | Intron 2 |
| 1032 | PGSC0003DMG400014966 | Intron 1 |
| 1033 | PGSC0003DMG400014966 | Intron 2 |
| 1034 | PGSC0003DMG400014966 | Intron 3 |
| 1035 | PGSC0003DMG400014966 | Intron 4 |
| 1036 | PGSC0003DMG400014966 | Intron 5 |
| 1037 | PGSC0003DMG400014966 | Intron 6 |
| 1038 | PGSC0003DMG400015180 | Intron 1 |
| 1039 | PGSC0003DMG400015180 | Intron 2 |
| 1040 | PGSC0003DMG400015180 | Intron 3 |
| 1041 | PGSC0003DMG400015180 | Intron 4 |
| 1042 | PGSC0003DMG400015180 | Intron 5 |
| 1043 | PGSC0003DMG400015180 | Intron 6 |
| 1044 | PGSC0003DMG400015180 | Intron 7 |
| 1045 | PGSC0003DMG400015180 | Intron 8 |
| 1046 | PGSC0003DMG400015180 | Intron 9 |
| 1047 | PGSC0003DMG400015180 | Intron 10 |
| 1048 | PGSC0003DMG400018449 | Intron 1 |
| 1049 | PGSC0003DMG400018449 | Intron 2 |
| 1050 | PGSC0003DMG400018449 | Intron 3 |
| 1051 | PGSC0003DMG400018449 | Intron 4 |
| 1052 | PGSC0003DMG400019204 | Intron 1 |
| 1053 | PGSC0003DMG400019204 | Intron 2 |
| 1054 | PGSC0003DMG400019204 | Intron 3 |
| 1055 | PGSC0003DMG400019204 | Intron 4 |
| 1056 | PGSC0003DMG400020244 | Intron 1 |
| 1057 | PGSC0003DMG400020244 | Intron 2 |
| 1058 | PGSC0003DMG400020244 | Intron 3 |
| 1059 | PGSC0003DMG400020244 | Intron 4 |
| 1060 | PGSC0003DMG400020244 | Intron 5 |
| 1061 | PGSC0003DMG400020244 | Intron 6 |
| 1062 | PGSC0003DMG400020244 | Intron 7 |
| 1063 | PGSC0003DMG400020244 | Intron 8 |
| 1064 | PGSC0003DMG400020244 | Intron 9 |
| 1065 | PGSC0003DMG400020244 | Intron 10 |
| 1066 | PGSC0003DMG400020244 | Intron 11 |
| 1067 | PGSC0003DMG400020244 | Intron 12 |
| 1068 | PGSC0003DMG400020244 | Intron 13 |
| 1069 | PGSC0003DMG400020850 | Intron 1 |
| 1070 | PGSC0003DMG400020850 | Intron 2 |
| 1071 | PGSC0003DMG400022148 | Intron 1 |
| 1072 | PGSC0003DMG400022148 | Intron 2 |
| 1073 | PGSC0003DMG400022148 | Intron 3 |
| 1074 | PGSC0003DMG400022148 | Intron 4 |
| 1075 | PGSC0003DMG400022148 | Intron 5 |
| 1076 | PGSC0003DMG400022148 | Intron 6 |
| 1077 | PGSC0003DMG400022148 | Intron 7 |
| 1078 | PGSC0003DMG400022148 | Intron 8 |
| 1079 | PGSC0003DMG400022148 | Intron 9 |
| 1080 | PGSC0003DMG400022148 | Intron 10 |
| 1081 | PGSC0003DMG400022148 | Intron 11 |
| 1082 | PGSC0003DMG400023429 | Intron 1 |
| 1083 | PGSC0003DMG400023429 | Intron 2 |
| 1084 | PGSC0003DMG400023429 | Intron 3 |
| 1085 | PGSC0003DMG400023429 | Intron 4 |
| 1086 | PGSC0003DMG400023708 | Intron 1 |
| 1087 | PGSC0003DMG400023708 | Intron 2 |
| 1088 | PGSC0003DMG400023708 | Intron 3 |
| 1089 | PGSC0003DMG400023708 | Intron 4 |
| 1090 | PGSC0003DMG400027746 | Intron 1 |
| 1091 | PGSC0003DMG400027746 | Intron 2 |
| 1092 | PGSC0003DMG400027746 | Intron 3 |
| 1093 | PGSC0003DMG400027746 | Intron 4 |
| 1094 | PGSC0003DMG400028193 | Intron 1 |
| 1095 | PGSC0003DMG400028193 | Intron 2 |
| 1096 | PGSC0003DMG400028193 | Intron 3 |
| 1097 | PGSC0003DMG400029120 | Intron 1 |
| 1098 | PGSC0003DMG400029120 | Intron 2 |
| 1099 | PGSC0003DMG400029746 | Intron 1 |
| 1100 | PGSC0003DMG400030319 | Intron 1 |
| 1101 | PGSC0003DMG400030319 | Intron 2 |
| 1102 | PGSC0003DMG400030319 | Intron 3 |
| 1103 | PGSC0003DMG400030319 | Intron 4 |
| 1104 | PGSC0003DMG400030431 | Intron 1 |
| 1105 | PGSC0003DMG400030431 | Intron 2 |
| 1106 | PGSC0003DMG400030627 | Intron 1 |
| 1107 | PGSC0003DMG400030627 | Intron 2 |
| 1108 | PGSC0003DMG400030627 | Intron 3 |
| 1109 | PGSC0003DMG402007428 | Intron 1 |
| 1110 | PGSC0003DMG402007428 | Intron 2 |
| 1111 | PGSC0003DMG402007428 | Intron 3 |
| 1112 | PGSC0003DMG402007428 | Intron 4 |
| 1113 | PGSC0003DMG402007428 | Intron 5 |
| 1114 | PGSC0003DMG402007428 | Intron 6 |
| 1115 | Zm00001eb000490 | Intron 1 |
| 1116 | Zm00001eb000800 | Intron 1 |
| 1117 | Zm00001eb000800 | Intron 2 |
| 1118 | Zm00001eb000800 | Intron 3 |
| 1119 | Zm00001eb000800 | Intron 4 |
| 1120 | Zm00001eb000800 | Intron 5 |
| 1121 | Zm00001eb000800 | Intron 6 |
| 1122 | Zm00001eb000800 | Intron 7 |
| 1123 | Zm00001eb000800 | Intron 8 |
| 1124 | Zm00001eb000800 | Intron 9 |
| 1125 | Zm00001eb000800 | Intron 10 |
| 1126 | Zm00001eb000800 | Intron 11 |
| 1127 | Zm00001eb000800 | Intron 12 |
| 1128 | Zm00001eb000800 | Intron 13 |
| 1129 | Zm00001eb000800 | Intron 14 |
| 1130 | Zm00001eb000800 | Intron 15 |
| 1131 | Zm00001eb000800 | Intron 16 |
| 1132 | Zm00001eb000800 | Intron 17 |
| 1133 | Zm00001eb000800 | Intron 18 |
| 1134 | Zm00001eb000800 | Intron 19 |
| 1135 | Zm00001eb009900 | Intron 1 |
| 1136 | Zm00001eb009900 | Intron 2 |
| 1137 | Zm00001eb009900 | Intron 3 |
| 1138 | Zm00001eb009900 | Intron 4 |
| 1139 | Zm00001eb009920 | Intron 1 |
| 1140 | Zm00001eb009920 | Intron 2 |
| 1141 | Zm00001eb009920 | Intron 3 |
| 1142 | Zm00001eb043800 | Intron 1 |
| 1143 | Zm00001eb043800 | Intron 2 |
| 1144 | Zm00001eb043800 | Intron 3 |
| 1145 | Zm00001eb043800 | Intron 4 |
| 1146 | Zm00001eb055330 | Intron 1 |
| 1147 | Zm00001eb055330 | Intron 2 |
| 1148 | Zm00001eb055330 | Intron 3 |
| 1149 | Zm00001eb063720 | Intron 1 |
| 1150 | Zm00001eb063720 | Intron 2 |
| 1151 | Zm00001eb063720 | Intron 3 |
| 1152 | Zm00001eb063720 | Intron 4 |
| 1153 | Zm00001eb079680 | Intron 1 |
| 1154 | Zm00001eb079680 | Intron 2 |
| 1155 | Zm00001eb079680 | Intron 3 |
| 1156 | Zm00001eb079680 | Intron 4 |
| 1157 | Zm00001eb079680 | Intron 5 |
| 1158 | Zm00001eb079680 | Intron 6 |
| 1159 | Zm00001eb079680 | Intron 7 |
| 1160 | Zm00001eb092070 | Intron 1 |
| 1161 | Zm00001eb092070 | Intron 2 |
| 1162 | Zm00001eb092070 | Intron 3 |
| 1163 | Zm00001eb095960 | Intron 1 |
| 1164 | Zm00001eb095960 | Intron 2 |
| 1165 | Zm00001eb095960 | Intron 3 |
| 1166 | Zm00001eb146780 | Intron 1 |
| 1167 | Zm00001eb146780 | Intron 2 |
| 1168 | Zm00001eb146780 | Intron 3 |
| 1169 | Zm00001eb146780 | Intron 4 |
| 1170 | Zm00001eb173290 | Intron 1 |
| 1171 | Zm00001eb173290 | Intron 2 |
| 1172 | Zm00001eb173290 | Intron 3 |
| 1173 | Zm00001eb173290 | Intron 4 |
| 1174 | Zm00001eb173290 | Intron 5 |
| 1175 | Zm00001eb173290 | Intron 6 |
| 1176 | Zm00001eb173290 | Intron 7 |
| 1177 | Zm00001eb202400 | Intron 1 |
| 1178 | Zm00001eb202400 | Intron 2 |
| 1179 | Zm00001eb202400 | Intron 3 |
| 1180 | Zm00001eb202400 | Intron 4 |
| 1181 | Zm00001eb215710 | Intron 1 |
| 1182 | Zm00001eb215710 | Intron 2 |
| 1183 | Zm00001eb215710 | Intron 3 |
| 1184 | Zm00001eb216070 | Intron 1 |
| 1185 | Zm00001eb216070 | Intron 2 |
| 1186 | Zm00001eb216070 | Intron 3 |
| 1187 | Zm00001eb216070 | Intron 4 |
| 1188 | Zm00001eb218000 | Intron 1 |
| 1189 | Zm00001eb218000 | Intron 2 |
| 1190 | Zm00001eb220480 | Intron 1 |
| 1191 | Zm00001eb220480 | Intron 2 |
| 1192 | Zm00001eb220480 | Intron 3 |
| 1193 | Zm00001eb220480 | Intron 4 |
| 1194 | Zm00001eb232910 | Intron 1 |
| 1195 | Zm00001eb232910 | Intron 2 |
| 1196 | Zm00001eb246220 | Intron 1 |
| 1197 | Zm00001eb246220 | Intron 2 |
| 1198 | Zm00001eb246220 | Intron 3 |
| 1199 | Zm00001eb246220 | Intron 4 |
| 1200 | Zm00001eb246220 | Intron 5 |
| 1201 | Zm00001eb246220 | Intron 6 |
| 1202 | Zm00001eb246220 | Intron 7 |
| 1203 | Zm00001eb246220 | Intron 8 |
| 1204 | Zm00001eb246220 | Intron 9 |
| 1205 | Zm00001eb267280 | Intron 1 |
| 1206 | Zm00001eb267280 | Intron 2 |
| 1207 | Zm00001eb267280 | Intron 3 |
| 1208 | Zm00001eb267280 | Intron 4 |
| 1209 | Zm00001eb267280 | Intron 5 |
| 1210 | Zm00001eb275020 | Intron 1 |
| 1211 | Zm00001eb275020 | Intron 2 |
| 1212 | Zm00001eb282650 | Intron 1 |
| 1213 | Zm00001eb282650 | Intron 2 |
| 1214 | Zm00001eb282650 | Intron 3 |
| 1215 | Zm00001eb282650 | Intron 4 |
| 1216 | Zm00001eb282650 | Intron 5 |
| 1217 | Zm00001eb282650 | Intron 6 |
| 1218 | Zm00001eb282650 | Intron 7 |
| 1219 | Zm00001eb282650 | Intron 8 |
| 1220 | Zm00001eb282650 | Intron 9 |
| 1221 | Zm00001eb282650 | Intron 10 |
| 1222 | Zm00001eb282650 | Intron 11 |
| 1223 | Zm00001eb331340 | Intron 1 |
| 1224 | Zm00001eb331340 | Intron 2 |
| 1225 | Zm00001eb331340 | Intron 3 |
| 1226 | Zm00001eb331340 | Intron 4 |
| 1227 | Zm00001eb331340 | Intron 5 |
| 1228 | Zm00001eb331340 | Intron 6 |
| 1229 | Zm00001eb331340 | Intron 7 |
| 1230 | Zm00001eb331340 | Intron 8 |
| 1231 | Zm00001eb331340 | Intron 9 |
| 1232 | Zm00001eb331340 | Intron 10 |
| 1233 | Zm00001eb331340 | Intron 11 |
| 1234 | Zm00001eb331340 | Intron 12 |
| 1235 | Zm00001eb331340 | Intron 13 |
| 1236 | Zm00001eb331340 | Intron 14 |
| 1237 | Zm00001eb335830 | Intron 1 |
| 1238 | Zm00001eb335830 | Intron 2 |
| 1239 | Zm00001eb335830 | Intron 3 |
| 1240 | Zm00001eb335830 | Intron 4 |
| 1241 | Zm00001eb335830 | Intron 5 |
| 1242 | Zm00001eb335830 | Intron 6 |
| 1243 | Zm00001eb335830 | Intron 7 |
| 1244 | Zm00001eb335830 | Intron 8 |
| 1245 | Zm00001eb335830 | Intron 9 |
| 1246 | Zm00001eb335830 | Intron 10 |
| 1247 | Zm00001eb335830 | Intron 11 |
| 1248 | Zm00001eb345620 | Intron 1 |
| 1249 | Zm00001eb345620 | Intron 2 |
| 1250 | Zm00001eb345620 | Intron 3 |
| 1251 | Zm00001eb345620 | Intron 4 |
| 1252 | Zm00001eb345620 | Intron 5 |
| 1253 | Zm00001eb345620 | Intron 6 |
| 1254 | Zm00001eb345620 | Intron 7 |
| 1255 | Zm00001eb345620 | Intron 8 |
| 1256 | Zm00001eb345620 | Intron 9 |
| 1257 | Zm00001eb345620 | Intron 10 |
| 1258 | Zm00001eb345620 | Intron 11 |
| 1259 | Zm00001eb348450 | Intron 1 |
| 1260 | Zm00001eb348450 | Intron 2 |
| 1261 | Zm00001eb348450 | Intron 3 |
| 1262 | Zm00001eb369310 | Intron 1 |
| 1263 | Zm00001eb369310 | Intron 2 |
| 1264 | Zm00001eb369310 | Intron 3 |
| 1265 | Zm00001eb411820 | Intron 1 |
| 1266 | Zm00001eb411820 | Intron 2 |
| 1267 | Zm00001eb411820 | Intron 3 |
| 1268 | Zm00001eb411820 | Intron 4 |
| 1269 | Zm00001eb411820 | Intron 5 |
| 1270 | Zm00001eb411820 | Intron 6 |
| 1271 | Zm00001eb411820 | Intron 7 |
| 1272 | Zm00001eb411820 | Intron 8 |
| 1273 | HORVU.MOREX.r3.4HG0337850.1 | Intron 1 |
| 1274 | HORVU.MOREX.r3.4HG0337850.1 | Intron 2 |

In various embodiments, non-coding regions such as the introns of genes described herein are used as “horses” to carry regulatory nucleic acids and/or nucleic acid sequences encoding small regulatory peptides. Upon transcription and mRNA splicing such regulatory nucleic acids can move to the cytoplasm of the cells where they can exert functions such as gene silencing of endogenous gene targets or gene targets from pests and disease-causing organisms or encodes small peptides with regulatory functions related to plant growth, development, acquisition of nutrients and water, or immunological response against pests and diseases. In some embodiments, the natural mRNA transcript from the gene that has a modified (e.g., genetically edited) intron, upon transcription and mRNA splicing, give rise to the natural mRNA of the said gene. The natural mRNA moves to the cytoplasm of the cell and is translated into the natural protein and thus, the natural function of the gene/protein is preserved.

### Nuclease Recognition Sites

Provided herein are cells comprising a non-coding region, e.g., a first intron region, that comprises a nuclease recognition site. In some embodiments, a nuclease is a CRISPR associated nuclease. In a particular embodiment, the CRISPR associated nuclease comprises Cas9. In other embodiments, a nuclease is a Transcription Activator-Like Effector Nuclease (TALEN).

Non-limiting examples of the nuclease recognition site are disclosed in Table 3. In some embodiments, the nuclease recognition sites may be, as for example, intronic sequences of the gene ACTIN 1 from rice, soybean, barley, tomato, sorghum, and maize. For example, some introns of ACTIN 1 or of ACTIN 1 homologue from six organisms are shown in Table 3. The nuclease (Cas9) recognition sequences guided by a guide RNA (sRNA) are as follow: 20 nucleotides (underlined) upstream of PAM motif (bold, underlined). The PAM motif is 3′NGG in the forward direction or 5′CCN in the reverse direction. The double strand DNA cleavage by Cas9 is 3 nucleotides before PAM comprising the underlined region.

TABLE 3
Examples of nuclease recognition site. The first column (SEQ ID NO)
contains the sequence identifier of non-limiting examples of nuclease recognition
site(s) of each endogenous introns of ACTIN 1 homologue from six organisms (SEQ ID
NOS: 1275-1284). The second column contains the organism and the code identifier
of the gene deposited in EnsemblPlants database described in Table 1, in addition
to the intron region of the corresponding gene. The third column contains the
sequence of the intron, with 20 nucleotides (underlined) upstream of PAM motif
(bold, underlined), necessary to the nuclease (Cas9) recognition.

| | Organism, gene | |
|---|---|---|
| SEQ ID | and intron | |
| NO | identification | Sequence of intron with nuclease recognition site(s) |
| 1275 | rice_Os03t0718100 | GTAAGCTGTTTGGATCTCAGGGTGGTTTCCGTTTAC |
| | -01 intron 1 | CGAAATGCTGCATTTCTTGGTAGCAAAACTGAGGT |
| | | GGTTTGTGTCAG |
| 1276 | rice_Os03t0718100 | GTGAGCACATTCGACACTGAACTAAAAGGCTGTGA |
| | -01 intron 2 | GGATGAATTTTAATTTTGACATTCACATGTAGATGA |
| | | GATTTAGTTCTGCAATCTTCAATTGTCATACAGCAA |
| | | GACTATATAATAGCTTTCAAAATAAAATCATAGGCA |
| | | GTTCTCATAAATGGAATCATGTTTGAACATCCTAAT |
| | | TCTGTTGGCATGGAGTGCTTTGACATTTTGATGTGC |
| | | TACAGTTGTGAATAACTGAATTTCCTTTTCCCAG |
| 1277 | soybean_GLYMA_ | GTAAGCAATTTTTTATTTGTTTGGTTAGCTTGAGGCT |
| | 02G091900 intron | GTATAGTTTGAACTTATCTGGGAACTTTGAAATTGA |
| | 2 | AATAAACACATGTCAACATCCATTAGCATCTATAAA |
| | | GTCTGTATGTTCAACATTGCATTTCCAGATATATTA |
| | | GAATAAACTGAAAATGATTCATTTCATTGTGAATGA |
| | | TTTCACTTGACATATTACTAAATGGGGAAGAAACAC |
| | | ATTCTGTGAACTGCATTGCATTTGGAAATTGTGATT |
| | | GTAGAAAACTTGTATAAAGCTTCCATATATCATGTG |
| | | ATTGACTTTATAAACTTCATTTATTTGCCTTCTTTTT |
| | | GCAG |
| 1278 | barley_HORVU. | GTAACGACCATCTCATGCCCCCCCTCCTCCTCCTCCC |
| | MOREX.r3.4HG0337850.1 | CGCCCCGATCGATCATTCTCTCCCCAGATGAGATCC |
| | intron 1 | GCCCCGCATTGCGCGCCCCCTTGCGCTCACCATCAG |
| | | GCCCGATCGATCCGATCTCGCGGCGGGCCCCGGCGC |
| | | CGGCTCCCCCTCTTCCCGCCGCGGATCCGACCGGAT |
| | | CTCGCGGAATGGGAGCGAGCGAGCGCCCGCCGCGT |
| | | TTTTTTTTATTTTCGTGCCGCCGCGGGAATGTAGAT |
| | | CTGCCGCCCGCGATCTGGGCCGTTTCGGGTGCCGG |
| | | AGCGCTCCGATCCGCGCCGCGGTGCCAGAGAACCA |
| | | TGCTCTCTCTGTTCCCTTATTAACTGTTCTTGTCGCT |
| | | AAGCCTCGCGGTTAACATGCCAGCCATTGCGGTTA |
| | | GGGGTTCCTCGTGAACTGATTGATAGTACTACTCAT |
| | | CATGTTTCTTCGAAGGAGAAAAATAATAATCTCTGC |
| | | ATTTATTTAGCCAGCGTCCTTCACTGCTAGGATGCG |
| | | TAGATTCTCCTTTGATCGACCGATCGATGTGTAATA |
| | | ATAACCGTGGCTTCCTGCGAGCTTCTGCTGTAG |
| 1279 | barley_HORVU. | GTGAGCCGCTGCATCCCACGAGCGTTCTTGCATCTT |
| | MOREX.r3.4HG0337850.1 | TGGTTCAGGGAAATGCCGCAGTGCTCTTTGTGACAT |
| | intron 2 | AACACTTGTGGTGGTGATGATGCTTGCAG |
| 1280 | tomato | GTAATCTCGACGAATTTAGAATTATATGAAAATGAT |
| | Solyc11g065990.2 | TCAATATCGTGTTTATTAATCGTTTAGTGTTTGGCAT |
| | intron 1 | GAATTTAGAATTTAAAATTGTATGAGAATGATTCAA |
| | | AATCGTGTCTATGGATATTGTAG |
| 1281 | sorghum_SORBI__ | GTAATGATTCGTTCACATGGTGACCAAATTTCATTA |
| | 3005G047100 | GGACCTCCAATTTTTTTAGCGCAAAGGACCTCCAAA |
| | intron 2 | ATTTTCACTCTTAATTATGGATTGCTGTGGTTCATTC |
| | | AG |
| 1282 | sorghum | GTTAAAAAACTTGCACTTTTTTGTTTGTTTCCAGTTG |
| | SORBI_3005G047100 | CTATGGCAAATATTGCAACCGTTCAGTAGTTCTAGT |
| | intron 3 | ATTTCTTTGTACTGAAAAAATATGCTCTGGTATTAT |
| | | TTTTGTACAG |
| 1283 | maize_ | GTGAGCTGATTCCGTTCTCTGCTTAACAATTTCCAA |
| | Zm00001eb216070_T001 | ATCCGATTTGCTCCTGCAGTCCGTTATCTCTAATATC |
| | intron 3 | TGTGGCTTCCCCTCTCTCAG |
| 1284 | maize_ | GTAAAACAAACAAACAAATTTCGCATTCGCATTTCC |
| | Zm00001eb216070_T001 | TCAATCAAGGTCTGCTCACGATTTTGCTTGCGACTG |
| | intron 4 | CAG |

In various embodiments using a CRISPR system, gRNA is used to guide the Cas protein (e.g., Cas9) to recognition sites for targeted cleavage. Non-limiting examples of gRNA are described in Table 4. The name of each gRNA sequence is a number representing the position into the respective intron sequence of Table 3. The position is followed by the orientation of PAM motif into the respective intron sequence of Table 3. (forw is in the forward orientation, rev is in the reverse orientation). In one aspect, the gRNA comprises 17-22 nucleotides in length (not considering the PAM motif), and 20-80% of GC content, and absence of TT-motif or GGC motif, and specificity score equal or superior to 80. In one aspect, the gRNA is a sequence wherein the corresponding cleavage site in the intron is distant at least 20 nucleotides from the intronic 5′ splice site (GU intron signal), and at least 45 nucleotides distant from the intronic 3′ splice site (AG intron signal), and out of the UA-rich element (a region of 4-7 nucleotides UA-rich, normally TTTTTAT present along the intronic regions of the gene).

TABLE 4
Examples of gRNAs. The second column describes the origin of non-
limiting examples of gRNAs (organism and the code identifier of the respective gene
from Table 3, in addition to the intron region of the correspondent gene). The third
column describes the name of each gRNA, comprising the position into the respective
intron sequence and the orientation of PAM motif. The four column contains the sequence
of each sRNA comprising 20 nucleotides upstream of PAM motif (bold), necessary to the
nuclease (Cas9) recognition. The first column (SEQ ID NO) contains the sequence
identifier of exemplified gRNA sequences (SEQ ID NOS: 1285-1315).

| SEQ | | gRNA name | |
|---|---|---|---|
| ID | | (position and | |
| NO | gRNA origin | orientation) | gRNA Sequence |
| 1285 | rice_Os03t0718100-01 intron 1 | 23forw | AAGCTGTTTGGATCTCAGGGTGG |
| 1286 | guide RNA sequences | 29rev | AAATGCAGCATTTCGGTAAACGG |
| 1287 | | 68forw | ATTTCTTGGTAGCAAAACTGAGG |
| 1288 | | 71forw | TCTTGGTAGCAAAACTGAGGTGG |
| 1289 | rice_Os03t0718100-01 intron 2 | 27forw | ACATTCGACACTGAACTAAAAGG |
| 1290 | guide RNA sequences | 35forw | CACTGAACTAAAAGGCTGTGAGG |
| 1291 | | 155forw | TCATAGGCAGTTCTCATAAATGG |
| 1292 | | 174rev | CACTCCATGCCAACAGAATTAGG |
| 1293 | | 185forw | TTTGAACATCCTAATTCTGTTGG |
| 1294 | | 190forw | ACATCCTAATTCTGTTGGCATGG |
| 1295 | soybean_GLYMA_02G091900 | 93rev | ACAGACTTTATAGATGCTAATGG |
| | intron 2 guide RNA sequences | | |
| 1296 | barley_HORVU.MOREX.r3. | 103rev | ATCGGATCGATCGGGCCTGATGG |
| 1297 | 4HG0337850.1 intron 2 guide | 234rev | GCAGATCTACATTCCCGCGGCGG |
| 1298 | RNA sequences | 237rev | GCGGCAGATCTACATTCCCGCGG |
| 1299 | | 268forw | TAGATCTGCCGCCCGCGATCTGG |
| 1300 | | 269forw | AGATCTGCCGCCCGCGATCTGGG |
| 1301 | | 294rev | TCTGGCACCGCGGCGCGGATCG |
| 1302 | | 363rev | TGGCTGGCATGTTAACCGCGAGG |
| 1303 | | 368forw | GTTCTTGTCGCTAAGCCTCGCGG |
| 1304 | | 379rev | GAACCCCTAACCGCAATGGCTGG |
| 1305 | | 394forw | ACATGCCAGCCATTGCGGTTAGG |
| 1306 | | 401rev | GTACTATCAATCAGTTCACGAGG |
| 1307 | | 546forw | TCGATGTGTAATAATAACCGTGG |
| 1308 | barley_HORVU.MOREX.r3. | 15rev | AAAGATGCAAGAACGCTCGTGGG |
| | 4HG0337850.1 intron 2 guide | | |
| | RNA sequence | | |
| 1309 | tomato_Solyc11g065990.2 | 121forw | ATGATTCAAAATCGTGTCTATGG |
| | intron 1 RNA sequence | | |
| 1310 | sorghum_ | 40rev | CTTTGCGCTAAAAAAATTGGAGG |
| | SORBI_3005G047100 intron 2 | | |
| 1311 | guide RNA sequence | 100forw | CTCTTAATTATGGATTGCTGTGG |
| 1312 | sorghum_SORBI_3005G047100 | 31rev | GCAATATTTGCCATAGCAACTGG |
| 1313 | intron 3 guide RNA sequences | 101forw | TGTACTGAAAAAATATGCTCTGG |
| 1314 | maize_Zm00001eb216070_T001 | 76forw | TCCGTTATCTCTAATATCTGTGG |
| | intron 3 guide RNA sequence | | |
| 1315 | maize_Zm00001eb216070_T001 | 35rev | TCGTGAGCAGACCTTGATTGAGG |
| | intron 4 guide RNA sequences | | |

In certain embodiments, the nuclease is fused to a VirD2 protein. VirD2 protein is one of the key proteins of Agrobacterium tumefaciens and involved in T-DNA processing and transfer. VirD2 contains an endonuclease domain as well as two nuclear localization signals (NLS), which can target marker proteins to the host-plant genome. VirD2 is tightly linked to the T-DNA by covalent binding and transported to the host-plant genome. In certain embodiments, the nuclease described herein may be fused to VirD2, thereby increasing the efficiency of integration of the non-coding, e.g., intron of the genes.

The nucleic acid sequence and amino acid sequence of VirD2 are described in Table 5. In some embodiments, the nuclease is fused to a VirD2 protein. In some embodiments, the amino acid sequence of VirD2 protein is at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the amino acid sequence of Table 5. In some embodiments, the sequence encoding the nuclease further comprises a sequence encoding VirD2. In some embodiments, the sequence encoding VirD2 is a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the nucleic acid sequence of Table 5.

TABLE 5
VirD2 sequences. The first column (SEQ ID NO) contains the sequence identifier
of an example of a sequence of VirD2 gene and its correspondent protein (SEQ ID
NOS: 1316-1317). The second column contains the information describing if the
sequence is a gene or a protein. The third column contains the sequence of an
example of VirD2 gene and its corresponding amino acid sequence.

| SEQ ID | | |
|---|---|---|
| NO | VirD2 | Sequence |
| 1316 | VirD2 open | ATGCCTGACAGAGCACAGGTAATCATACGGATTGTTCCTGGAGGAGGC |
| | read frame | ACCAAAACGCTGCAACAGATCATCAACCAACTTGAGTATTTGAGCAGG |
| | | AAAGGAAAACTAGAGCTTCAGCGATCTGCAAGACACCTGGACATTCCT |
| | | GTACCTCCCGATCAGATCAGAGAGTTAGCACAATCATGGGTTACAGAG |
| | | GCTGGCATTTACGATGAGTCTCAATCTGATGACGACAGGCAGCAAGAT |
| | | CTGACGACACATATCATTGTCTCCTTCCCAGCGGGGACAGATCAAACT |
| | | GCCGCTTATGAGGCCAGCCGAGAATGGGCTGCAGAGATGTTTGGAAGT |
| | | GGTTATGGTGGGGGGCGCTACAACTACTTGACCGCTTACCATGTTGAT |
| | | AGAGATCATCCACACTTGCACGTCGTAGTGAATAGAAGGGAACTCCTT |
| | | GGCCAAGGATGGCTTAAAATTTCGCGCCGGCATCCTCAGTTGAATTAT |
| | | GATGGTCTCCGTAAGAAGATGGCTGAGATCTCACTCCGTCACGGAATT |
| | | GTGTTAGATGCTACTTCCCGAGCAGAAAGAGGGATTGCTGAGAGGCCC |
| | | ATAACATATGCTGAATACAGAAGATTAGAAAGAATGCAAGCTCAGAA |
| | | GATTCAGTTTGAAGACACTGATTTTGATGAAACATCACCAGAAGAGGA |
| | | TCGCAGGGATCTTTCTCAGTCTTTCGATCCTTTCAGGAGTGATGCATCA |
| | | GCCGGAGAACCCGACCGAGCTACTAGACACGACAAACAACCACTTGA |
| | | GCCTCATGCAAGATTCCAAGAACCTGCTGGTTCCTCTATCAAAGCTGAT |
| | | GCCCGAATAAGGGTTCCGTTGGAGTCTGAGAGAGGGGCGCAGCCATCA |
| | | GCGTCCAAGATACCAGTGACCGGTCATTTTGGTATTGAAACTTCTTATG |
| | | TGGCTGAAGCATCAGTTCCGAAGCAGAGTGGAAATTCAGACACAAGC |
| | | AGACCAGTCACGGATGTTGCTATGCATACTGTGGAGCGGCAACAAAGA |
| | | TCAAAGAGAAGGCATGATGAAGAAGCTGGACCGTCGGGCGCCAATCG |
| | | GAAACGTCTCAAGGCGGCTCAAGTGGACTCTGAAGCAAATGTTGGTGA |
| | | ACCGGATGGAAGAGACGATTCGAACAAAGCGGCAGATCCAGTTAGTG |
| | | CTAGTATAAGAACAGAACAACCTGAAGCAAGCCCGACCTGTCCTCGTG |
| | | ATCGTCATGACGGTGAGCTAGGGGAGCGTAAGAGAGCTAGGGGAAAC |
| | | AGACGAGATGATGGAAGGGGTGGTACTTGA |
| 1317 | VirD2 | MPDRAQVIIRIVPGGGTKTLQQIINQLEYLSRKGKLELQRSARHLDIPVPPD |
| | protein | QIRELAQSWVTEAGIYDESQSDDDRQQDLTTHIIVSFPAGTDQTAAYEASR |
| | | EWAAEMFGSGYGGGRYNYLTAYHVDRDHPHLHVVVNRRELLGQGWLKI |
| | | SRRHPQLNYDGLRKKMAEISLRHGIVLDATSRAERGIAERPITYAEYRRLE |
| | | RMQAQKIQFEDTDFDETSPEEDRRDLSQSFDPFRSDASAGEPDRATRHDK |
| | | QPLEPHARFQEPAGSSIKADARIRVPLESERGAQPSASKIPVTGHFGIETSYV |
| | | AEASVPKQSGNSDTSRPVTDVAMHTVERQQRSKRRHDEEAGPSGANRKR |
| | | LKAAQVDSEANVGEPDGRDDSNKAADPVSASIRTEQPEASPTCPRDRHDG |
| | | ELGERKRARGNRRDDGRGGT |

### Endogenous and Exogenous Nucleic Acids

Provided herein, in certain embodiments, are cells comprising an endogenous or exogenous nucleic acid in a non-coding region such as an intron (e.g., modified intron region), a 5′ non-coding region, or a 3′ non-coding region. In certain aspects, the endogenous or exogenous nucleic acid is transcribed from a native promotor of the gene comprising the non-coding region, e.g., intron. In some embodiments, the promotor is a constitutive promotor. In one aspect, the promotor is specific for a plant organ. Examples of the plant organ include, not limited to, a root, stem, fruit, seed, and leaf. In another aspect, the promoter is specific for a plant tissue. Examples of the plant tissue include, but not limited to, a ground tissue, vascular tissue, and dermal tissue. In certain aspects, the promoter is an endogenous promoter of a gene encoding a native protein. In some embodiments, the promoter drives the expression of the genes described in Table 1. The endogenous or exogenous nucleic acid may be exogenous to the cell. The endogenous or exogenous nucleic acid may be exogenous to the non-coding region. The endogenous or exogenous nucleic acid may be endogenous to the cell. The endogenous or exogenous nucleic acid may be endogenous to the cell, and exogenous to the non-coding region. The endogenous or exogenous nucleic acid may be endogenous to the non-coding region.

In some embodiments, the endogenous or exogenous nucleic acid is at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 or more bases in length (e.g., up to about 700 bases in length). In some embodiments, the endogenous or exogenous nucleic acid is fewer than 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 bases in length. In some embodiments, the endogenous or exogenous nucleic acid is about 10 to about 700, about 10 to about 200, about 10 to about 180, about 10 to about 160, about 10 to about 140 bases, about 10 to about 120 bases, about 10 to about 110 bases, or about 10 to about 100 bases in length. In some embodiments, the exogenous nucleic acid is less than 700, 600, 500, 400, 300, 280, 260, 240, 220, 200, 180, 160, 140, 120, 100, 80, 60, 40, 20 bases in length. In some embodiments, the exogenous nucleic acid is positioned within the genome of the cell. In some embodiments, the exogenous nucleic acid is not present on a plasmid.

miRNAs

In one aspect, the endogenous or exogenous nucleic acid encodes a microRNA (miRNA). In some embodiments, the exogenous miRNA is an artificial microRNA (amiRNA). miRNA is a small single-stranded RNA that functions in RNA silencing and post-transcriptional regulation. miRNA contains complementary base pairs to its target mRNA molecule, thereby repressing gene expression of the target mRNA. As a result, the target mRNA is silenced via one of the following processes: breakdown of the mRNA strand, destabilization of the mRNA through shortening of its polyA, and inefficient translation of the mRNA into proteins. In various embodiments, a regulatory nucleic acid to be inserted into the non-coding region, e.g., intron of genes, described herein may be a micro-RNA (mi-RNA) or an artificial micro-RNA (amiRNA). Upon mRNA transcription and splicing, such miRNA or amiRNA moves into the cell cytoplasm and silence the target gene.

In some embodiments, the endogenous or exogenous miRNA is a precursor miRNA. In other embodiments, the endogenous or exogenous miRNA is a mature miRNA. In some embodiments, the mature miRNA comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleotides. In some embodiments, miRNA comprises about 21-22 nucleotides. In some embodiment, the miRNA can be expressed as short tandem target mimic (STTM), which harbors two copies of small RNA (e.g., 10-30 nucleotides) partially complementary sequences linked by a short spacer. Designed spacers can be with different lengths such as about 6 to about 60 nucleotides (e.g., 8, 31, to 48 nucleotides).

In some embodiments, the miRNA specifically binds to a target nucleic acid. In some embodiments, the target nucleic acid is endogenous and/or exogenous to the cell. Non-limiting examples of the target nucleic acid endogenous and/or exogenous to the cell are described in Table 6. In some embodiments, the target nucleic acid is from an insect and/or worm that is harmful to the cell. Non-limiting examples of the insect and/or worm are described in Table 6. Non-limiting examples of the target nucleic acid from the insect and/or worm are described in Table 6. In other embodiments, the target nucleic acid is from an organism that causes a disease to the cell. Non-limiting examples of the organism are described in Table 6. Non-limiting examples of the target nucleic acid from the organism that causes a disease to the cell are described in Table 6.

In various embodiments, the target nucleic acid is a target mRNA. In some embodiments, the target mRNA comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or is 100% identical to a sequence of Table 6. In one aspect, the target mRNA encodes for a target gene. Non-limiting examples of the target gene are described in Table 6. In some embodiments, the target gene comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or is 100% identical to a sequence of Table 6. In some embodiments, the endogenous or exogenous miRNA comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or is 100% identical to a sequence of Table 6.

TABLE 6
Examples of target nucleic acids. The first column contains non-limiting target pests (binomial
scientific names and organisms). The second column contains non-limiting target gene(s)
of each example target pest. The sequence of each target gene on second column is available
in the formal sequence listing filed herewith by reference to the SEQ ID NO in parenthesis
(SEQ ID NOS: 1318-1460) and defines the gene sequence described in the table. The third
column describes examples of hosts (crops and other plants) of the target pest.

| Target pest | Target gene(s) (SEQ ID NO) | Hosts (example) |
|---|---|---|
| Pseudomonas syringae | corP (1318), AvrB (1319), | Wide range, cereal crops |
| (bacterium) | HopBB1 (1320), HopX1 (1321), | |
| | HopZ1a (1322) | |
| Ralstonia solanacearum | PopP2 (1323), RipI (1324) | Wide range |
| (bacterium) | | |
| Xanthomonas oryzae pv. oryzae | Xopr (1325), PthXo1 (1326), | Wide range |
| (bacterium) | PthXo2 (1327), PthXo3 (1328), | |
| | AvrXa7 (1329), TalC (1330), | |
| | TALE1 (1331), TALE2 (1332), | |
| | TALE3 (1333), TALE7 (1334), | |
| | TALE12 (1335) | |
| Xanthomonas translucens pv. | tal8 (1336) | Wide range |
| undulosa | | |
| (bacterium) | | |
| Xanthomonas campestris pathvars | AvrXccE1 (1337), AvrXccB | Wide range |
| (bacterium) | (1338), XopE2 (1339), XopJ | |
| | (1340) | |
| Clavibacter michiganensis | chpC (1341) | tomato |
| (bacterium) | | |
| Erwinia amylovora | HrpN (1342) | fruit trees, ornamentals, |
| (bacterium) | | bushes |
| Phakopsora pachyrhizi | CSEP-07 (1343), CSEP-09 | soybean |
| (fungus) | (1344), acetyl-CoA | |
| | acyltransferase (1345), 40S | |
| | ribosomal protein S16 (1346), | |
| | Glycine cleavage system | |
| | H protein (1347) | |
| Ustilago maydis | Cmu1 (1348) | maize |
| (fungus) | | |
| Magnaporthe oryzae | AvrPi9 (1349), AVR-Pita1 | rice |
| (fungus) | (1350), AVR-Pita2 (1351), Pwl1 | |
| | (1352), Pwl2 (1353) | |
| Cladosporium fulvum | Avr4 (1354) | tomato |
| (fungus) | | |
| Botrytis cinerea | BCG1 (1355) | Wide range |
| (fungus) | | |
| Puccinia spp. | Avirulence factor (1356), | Wheat |
| (fungus) | AvrSr35 (1357) | |
| Fusarium oxysporum | Fmk1 (1358), rho1 (1359) | Tomato, melon, cotton, |
| (fungus) | | banana |
| Fusarium graminearum | CYP51A (1360), CYP51B | barley |
| (fungus) | (1361), CYP51C (1362) | |
| Phytophthora infestans | Avrblb2 (1363), AVR3a (1364) | Potato, tomato |
| (oomycete) | | |
| Phytophthora ramorum | RXLR (1365) | hardwood trees, |
| (oomycete) | | ornamentals |
| Phytophthora sojae | PsojNIP (1366), RXLR (1367), | soybean |
| (oomycete) | CRN114 (1368) | |
| Phytophthora capsica | CRN (1369) | Pepper, tomato, lima, |
| (oomycete) | | snap beans, cucurbit |
| Phytophthora parasitica | CBEL (1370), NPP1 (1371) | Wide range |
| (oomycete) | | |
| Meloidogyne spp. | Calreticulin (1372), NodL (1373), | Wide range |
| (nematode) | GPCR (1374), collagen (1375), | |
| | 14-3-3 (1376), Cysteine protease | |
| | (1377), gsts-1 (1378), Venom | |
| | allergen-like protein (1379) | |
| Heterodera spp. | CLAVATA3 (1380), Annexin | Soybean, Potato, sugar |
| (nematode) | 4C10 (1381), Annexin Hs4F01 | beet |
| | (1382), Transthyretin-like protein | |
| | precursor (1383), Ubi1 (1384), | |
| | Venom allergen-like protein | |
| | (1385) | |
| Pratylenchus spp. | pat-10 (1386), unc-87 | crop and ornamental |
| (nematode) | (1387), beta-1,4-endoglucanase | plants |
| | (1388) | |
| Radopholus similis | Transthyretin-like protein (1389), | Banana, citrus crops, |
| (nematode) | xyl1 (1390) | pepper |
| Helicoverpa armigera | Rack1 (1391), GAPDH (1392), | Soybean, maize, cotton, |
| (insect) | chitinase (1393) | wide range |
| Anticarvsia gemmatalis | GAPDH (1394) | Soybean, wide range |
| (insect) | | |
| Spodoptera frugiperda | chitinase (1395), cuticular protein | Maize, soybean, cotton, |
| (insect) | (1396) | tobacco, wheat, cassava |
| Chrysodeixis includens | Actin (1397), GAPDH (1398) | Soybean, wide range |
| (insect) | | |
| Diabrotica virgifera | chitinase 10 (1399), chitinase 2 | maize |
| (insect) | (1400), GAPDH (1401) | |
| Leptinotarsa decemlineata | PSMB5 (1402) | potato |
| (insect) | | |
| Bemisia tabaci | TLR7 (1403) | Bean, Wide range |
| (insect) | | |
| Myzus persicae | MpC002 (1404), cuticular protein | Potato, tomato |
| (insect) | (1405), GAPDH (1406), V- | |
| | ATPase-A (1407) | |
| Nephotettix cincticeps | NcSP75 (1408) | rice |
| (insect) | | |
| Tobacco mosaic virus | CP (1409), MP (1410), | tobacco |
| (virus) | | |
| Tomato spotted wilt virus | CP (1411) | Wide range, including |
| (virus) | | tomato, pepper, lettuce, |
| | | peanut, chrysanthemum |
| Tomato yellow leaf curl virus | CP (1412), MP (1413), Rep | Tomato, common bean, |
| (virus) | (1414), TrAP (1415), REn (1416), | sweet pepper, chilli |
| | C4 (1417) | pepper, tobacco |
| | | ornamentals, common |
| | | weeds |
| Cucumber mosaic virus | CP (1418), 2b supressor (1419), | Wide range, including |
| (virus) | 3a (1420), 1a (1421), 2a (1422) | tomato, pepper, melon |
| Potato virus Y | CP (1423) | Potato, tobacco, tomato, |
| (virus) | | pepper |
| African cassava mosaic virus | CP (1424), AV1 (1425), AV2 | cassava |
| (virus) | (1426), | |
| | AC1 (1427), AC2 (1428), AC3 | |
| | (1429), Rep (1430), Trap (1431), | |
| | AC4 (1432), BC1 (1433), BV1 | |
| | (1434) | |
| Plum pox virus | CP (1435), HC-Pro (1436) | stone fruit crops |
| (virus) | | |
| Potato virus X | TGBp1 (1437), TGBp2 (1438), | potato |
| (virus) | TGBp3 (1439), CP (1440) | |
| Citrus tristeza virus | CP (1441), p6 (1442), p65 (1443) | citrus |
| (virus) | p61 (1444), p20 (1445), p23 | |
| | (1446), p33 (1447), p18 (1448) | |
| | p13 (1449) | |
| Barley yellow dwarf virus | polymerase (1450), CP (1451), | Most grasses, oats, |
| (virus) | RTD (1452), | barley, wheat, maize, |
| | | rice |
| Potato leafroll virus | CP (1453), NSP (1454), MP | potato, tomato |
| (virus) | (1455) | |
| Bean golden mosaic virus | CP (1456), AC4 (1457), REP | bean |
| (virus) | (1458), TRAP (1459), REN | |
| | (1460) | |

### Small Peptides

In another aspect, the endogenous or exogenous nucleic acid encodes a peptide. In some embodiments, the peptide affects a property of the cell. Examples of the property of the cell include, but are not limited to, hormonal regulation, protection against a pathogen, protection against an insect, nitrogen fixation, nutrient acquisition, immunity induction, biotic stress, and abiotic stress.

In various embodiments, a regulatory nucleic acid to be inserted into the non-coding region, e.g., intron of the genes, described herein can be a nucleic acid sequence encoding a regulatory small peptide. Upon mRNA transcription and splicing, such nucleic acid sequence give rise to a mature mRNA that moves into the cell cytoplasm and is translated into a regulatory small peptide.

Non-limiting examples of the peptide and their biological functions thereof are described in Table 7.

TABLE 7
Non-limiting examples of small peptides. The first column
(Peptide Name) contains the name of non-limiting examples
of small peptides. The second column (Mature peptide sequence
length) contains the information of the length of the mature
small peptide in number of amino acid. The third column (Biological
function) contains the information of the known biological
function of the referred small peptide.

| Peptide | Mature peptide | |
|---|---|---|
| Name | sequence length (aa) | Biological function(s) |
| CEP1 | 15 | (Lateral) root development |
| CLE3 | 12-13 | Meristem maintenance, stem-cell |
| | | division, vascular development |
| IDA-IDL | 20 | Organ abscission, lateral root |
| | | emergence |
| PSK-α | 5 | Cell proliferation and differentiation, |
| | | cell expansion |
| PSY1 | 16-18 | Cell proliferation and differentiation, |
| | | cell expansion |
| RGF1 | 13 | Maintenance of root apical meristem |
| | | stem-cell niche, gravitropism, lateral |
| | | root development |

In some embodiments, a coding region of the peptide may be flanked by 5′ ribosomal binding site (RBS). In some embodiments, the RBS is at least 1, 2, 3, 4, 5, 6, 7, 8, 9, 10, 11, 12, 13, 14, 15, 16, 17, 18, 19, 20 or more base pair in length. In some embodiments, the RBS is about 1-50, 2-40, 3-30, 4-20, 5-10 base pair in length.

In some embodiments, the peptide is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acids in length. In some embodiments, the peptide is about 1-10, 1-20, 1-30, 2-10, 2-20, 2-30, 3-10, 3-20, 3-30, 4-10, 4-20, 4-30, 5-10, 5-20, 5-30, 6-10, 6-20, 6-30, 7-10, 7-20, 7-30, 8-10, 8-20, 8-30, 9-10, 9-20, 9-30, 10-20, or 10-30 amino acids in length. In some embodiments, the peptide is about 2-80, 3-80, 4-80, 5-80, 6-80, 7-80, 8-80, 9-80, 10-80, 20-80, 30-80, 40-80, 50-80, 60-80, 70-80 or 1-80 amino acids in length. In some embodiments, the peptide comprises a sequence at least 80% identical to a sequence of Table 8.

TABLE 8
Non-limiting examples of nucleic acid sequences encoding small peptides
The first column (SEQ ID NO) contains the sequence identifier of non-limiting examples
of small peptides (SEQ ID NOs 1461-1466). The second column (Peptide Name) contains the
name of non-limiting examples of small peptides. The third column (Mature peptide
sequence length) contains the information of the length of the mature small peptide in
number of amino acids. The fourth column (Precursor peptide sequence length) contains
the information of the length of the mRNA of the precursor peptide in number of
nucleotides. The fifth column (mRNA sequence) contains the acid nucleic sequence of
the mRNA of the referred precursor peptide.

| | | Mature | | mRNA of | |
|---|---|---|---|---|---|
| | | peptide | Precursor | precursor | |
| | | sequence | peptide | peptide | |
| SEQ ID | Peptide | length | sequence | sequence | |
| NO | name | (aa) | length (aa) | length (nt) | mRNA sequence |
| 1461 | CEP1 | 15 | 91 | 276 | ATGGGAATGTCGAATAGGTCAGTTTCT |
| | | | | | ACATCCATTTTTTTCCTTGCATTGGTGG |
| | | | | | TTTTGCATGGAATTCAGGACACAGAAG |
| | | | | | AGAGACATTTGAAAACTACTTCGTTAG |
| | | | | | AGATTGAGGGAATTTATAAAAAAACTG |
| | | | | | AGGCCGAGCATCCTAGCATTGTGGTCA |
| | | | | | CATATACACGGCGTGGTGTCCTTCAGA |
| | | | | | AGGAGGTCATTGCCCACCCCACAGACT |
| | | | | | TTAGGCCAACAAATCCCGGAAACAGCC |
| | | | | | CAGGCGTTGGACACTCTAACGGGCGAC |
| | | | | | ATTGA |
| 1462 | CLE3 | 12-13 | 96 | 291 | ATGGATTCGAAGAGTTTTCTGCTACTAC |
| | | | | | TACTACTCTTCTGCTTCTTGTTCCTTCAT |
| | | | | | GATGCTTCTGATCTCACTCAAGCTCATG |
| | | | | | CTCACGTTCAAGGACTTTCCAACCGCA |
| | | | | | AGATGATGATGATGAAAATGGAAAGTG |
| | | | | | AATGGGTTGGAGCAAATGGAGAAGCAG |
| | | | | | AGAAGGCAAAGACGAAGGGTTTAGGA |
| | | | | | CTACATGAAGAGTTAAGGACTGTTCCTT |
| | | | | | CGGGACCTGACCCGTTGCACCATCATG |
| | | | | | TGAACCCACCAAGACAGCCAAGAAACA |
| | | | | | ACTTTCAGCTCCCTTGA |
| 1463 | IDA- | 20 | 77 | 234 | ATGGCTCCGTGTCGTACGATGATGGTTC |
| | IDL | | | | TGCTCTGTTTTGTTCTGTTTCTCGCGGC |
| | | | | | GAGTAGTTCTTGTGTAGCGGCTGCAAG |
| | | | | | AATTGGAGCCACCATGGAGATGAAGAA |
| | | | | | GAATATAAAGAGATTAACGTTTAAAAA |
| | | | | | CAGCCATATTTTTGGTTACTTACCTAAA |
| | | | | | GGCGTTCCCATTCCTCCTTCTGCTCCTT |
| | | | | | CTAAGAGACACAACTCTTTTGTTAACTC |
| | | | | | TCTTCCTCATTGA |
| 1464 | PSK-α | 5 | 87 | 264 | ATGATGAAGACGAAAAGTGAAGTGTTG |
| | | | | | ATCTTTTTCTTCACTCTAGTATTGCTTTT |
| | | | | | AAGCATGGCTTCAAGTGTTATTTTAAGA |
| | | | | | GAAGATGGTTTTGCTCCTCCTAAACCAT |
| | | | | | CTCCCACCACACATGAGAAAGCAAGTA |
| | | | | | CTAAAGGTGACAGAGATGGAGTAGAGT |
| | | | | | GCAAGAATTCAGACAGTGAAGAAGAAT |
| | | | | | GTCTTGTGAAGAAAACAGTAGCTGCTC |
| | | | | | ACACCGATTACATCTATACACAAGATTT |
| | | | | | AAACCTATCTCCTTGA |
| 1465 | PSY1 | 16-18 | 75 | 228 | ATGACTTTTGTAGTTCGTCTTCTTGTGT |
| | | | | | GTCTCTTATTGACGCTTACAATTACATC |
| | | | | | TTCTCTAGCCCGCAACCCTGTTTCCGTT |
| | | | | | TCAGGTGGGTTTGAGAATTCTGGATTCC |
| | | | | | AAAGGAGTTTGTTGATGGTGAACGTTG |
| | | | | | AGGACTACGGTGACCCATCTGCAAATC |
| | | | | | CTAAGCACGACCCCGGCGTTCCTCCGTC |
| | | | | | AGCAACCGGCCAACGTGTCGTCGGCAG |
| | | | | | AGGCTGA |
| 1466 | RGF1 | 13 | 116 | 442 | AAAACACACAAGTTTACTCTTTTCTGTT |
| | | | | | CATATACGTACATCAAGCCAAGGAGAA |
| | | | | | AAAAGGAAGGCGAGATGGTGTCCATAA |
| | | | | | GGGTTATTTGCTATCTTTTAGTATTTTCC |
| | | | | | GTTTTGCAGGTGCATGCTAAAGTCTCCA |
| | | | | | ATGCAAACTTTAATAGCCAAGCTCCAC |
| | | | | | AAATGAAAAATAGTGAAGGTCTTGGAG |
| | | | | | CAAGCAATGGTACCCAAATTGCCAAGA |
| | | | | | AGCATGCTGAAGATGTAATTGAAAACC |
| | | | | | GAAAGACGTTGAAGCATGTAAATGTGA |
| | | | | | AGGTGGAGGCAAATGAGAAGAATGGTT |
| | | | | | TAGAAATAGAGAGTAAAGAAATGGTGA |
| | | | | | AGAAAAGAAAAAACAAGAAGAGACTC |
| | | | | | ACCAAGACGGAGAGTTTAACTGCCGAT |
| | | | | | TATAGCAACCCTGGTCATCATCCTCCTA |
| | | | | | GGCATAACTAAAACATATATATATATA |
| | | | | | TATATA |

### Host

In various embodiments, the cell is a plant cell. In some embodiments, the plant is a monocotyledonous plant. Non-limiting examples of the monocotyledonous plants are described in Table 9. In other embodiments, the plant is a dicotyledonous plant. Non-limiting examples of the dicotyledonous plant are described in Table 9.

TABLE 9
Non-limiting examples of monocotyledonous and dicotyledonous plants.
The first column (Monocot Plant) contains the binomial scientific
name of non-limiting examples of monocotyledonous plants that can
be modified (e.g., genetically edited) by the present platform.
The second column (Dicot Plant) contains the scientific name of
non-limiting examples of dicotyledonous plants that can be genetically
edited by the present modified by the present platform.

| Monocot Plant | Dicot Plant |
|---|---|
| Triticum aestivum | Arabidopsis lyrata |
| Triticum turgidum | Arabidopsis thaliana |
| Triticum dicoccoides | Capsella rubella |
| Aegilops tauschii | Cardamine hirsuta |
| Hordeum vulgare | Brassica carinata |
| Secale cereale | Brassica oleracea |
| Lolium perenne | Brassica rapa |
| Brachypodium distachyon | Brassica napus |
| Phyllostachys edulis | Schrenkiella parvula |
| Oryza alta | Eutrema salsugineum |
| Oryza sativa | Aethionema arabicum |
| Oryza brachyantha | Tarenaya hassleriana |
| Setaria italica | Carica papaya |
| Setaria viridis | Corchorus olitorius |
| Cenchrus purpureus | Theobroma cacao |
| Panicum hallii | Gossypium hirsutum |
| Digitaria exilis | Gossypium raimondii |
| Miscanthus sinensis | Durio zibethinus |
| Saccharum spontaneum | Acer truncatum |
| Sorghum bicolor | Citrus clementina |
| Zea mays | Eucalyptus grandis |
| Eragrostis curvula | Punica granatum |
| Oropetium thomaeum | Corylus avellana |
| Zoysia japonica ssp. nagirizaki | Carpinus fangiana |
| Ananas comosus | Carya illinoinensis |
| Musa acuminata | Quercus lobata |
| Calamus simplicifolius | Cucumis melo |
| Elaeis guineensis | Cucumis sativus L. |
| Asparagus officinalis | Citrullus lanatus |
| Allium sativum | Sechium edule |
| Vanilla planifolia | Fragaria × ananassa |
| Dioscorea dumetorum | Fragaria vesca |
| Spirodela polyrhiza | Rosa chinensis |
| Zostera marina | Malus domestica |
| | Prunus persica |
| | Cannabis sativa |
| | Medicago truncatula |
| | Trifolium pratense |
| | Pisum sativum |
| | Cicer arietinum L. |
| | Lotus japonicus |
| | Glycine max |
| | Phaseolus vulgaris |
| | Vigna mungo |
| | Lupinus albus |
| | Arachis hypogaea |
| | Manihot esculenta |
| | Sapria himalayana |
| | Populus trichocarpa |
| | Salix brachista |
| | Triptery gium wilfordii |
| | Vitis vinifera |
| | Rhododendron simsii |
| | Vaccinium macrocarpon |
| | Actinidia chinensis |
| | Camellia sinensis |
| | Davidia involucrata |
| | Hydrangea macrophylla |
| | Erythranthe guttata |
| | Striga asiatica |
| | Salvia bowleyana |
| | Utricularia gibba |
| | Avicennia marina |
| | Olea europaea |
| | Capsicum annuum |
| | Solanum lycopersicum |
| | Solanum pennellii |
| | Solanum tuberosum |
| | Nicotiana tabacum |
| | Petunia axillaris |
| | Coffea canephora |
| | Erigeron canadensis |
| | Helianthus annuus |
| | Lactuca sativa |
| | Daucus carota |
| | Lonicera japonica |
| | Beta vulgaris |
| | Chenopodium quinoa |
| | Amaranthus hybridus |
| | Selenicereus undatus |
| | Simmondsia chinensis |
| | Trochodendron aralioides |
| | Nelumbo nucifera |
| | Aquilegia oxysepala |
| | Papaver somniferum |
| | Ceratophyllum demersum |
| | Magnolia biondii |

In one aspect, the plant cell is a ground tissue cell. Examples of the tissue cell include, but not limited to, a parenchyma, collenchyma, and sclerenchyma cell. In another aspect, the plant cell is a vascular tissue cell. Examples of the vascular tissue cell include, but not limited to, a tracheid, vessel element, sieve tube cell, and companion cell. In yet another aspect, the plant cell is a dermal tissue cell. Examples of the dermal tissue cell include, but not limited to, an epidermal, guard cell, and trichome. In certain embodiments, the cell is not transgenic.

Also provided herein are seeds from the plant described herein. Further provided herein are plants obtained from the seed described herein.

In various embodiments, the plant has a trait. Examples of the trait include, but not limited to, hormonal regulation, protection against a pathogen, protection against an insect, nitrogen fixation, nutrient acquisition, immunity induction, biotic stress, and abiotic stress. In some embodiments, the trait is determined by regulatory nucleic acids or peptides for hormonal regulation, protection against a pathogen, protection against an insect, nitrogen fixation, nutrient acquisition, immunity induction, biotic stress, and abiotic stress.

In one aspect, the trait confers resistance to a pest and/or a disease caused by insects, microorganism and/or worms. Non-limiting examples of the pest are described in Table 6. In some embodiments, the resistance is due to antibiosis (growth and multiplication of the pest is inhibited), antixenosis (the pest is repelled by the plant), or tolerance (plant is able to withstand or recover from damage by the pest).

In another aspect, the trait confers resistance to a chemical. In some embodiments, the chemical is a weed control chemical. In a particular embodiment, the weed control chemical is a grown inhibitor. In other embodiments, the chemical is a herbicide. Examples of the herbicide include, but not limited to, 2,4-D (2,4-dichlorophenoxy acetic acid), Aminopyralid, Atrazine, Clopyralid, Dicamba, Glufosinate ammonium, Fluazifop, Fluroxypyr, Glyphosate, Imazapyr, Imazapic, Imazamox, Linuron, MCPA (2-methyl-4-chlorophenoxyacetic acid), Metolachlor, Paraquat, Pendimethalin, Picloram, Sodium chlorate, Triclopyr, Sulfonylureas (e.g., Flazasulfuron and Metsulfuron-methyl), and any other commercial herbicide.

In yet another aspect, the trait confers a nutritionally improved quality as compared to a plant that does not comprise the cell described herein. In some embodiments, the trait confers an increase in crop yield as compared to a plant that does not comprise the cell described herein. In some embodiments, the trait confers an ability to acquire nutrients more efficiently as compared to a plant that does not comprise the cells described herein. For example, the trait may increase the ability of the plant to acquire nutrients by at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold or more. In some embodiments, the trait confers an ability to acquire water more efficiently as compared to a plant that does not comprise the cells described herein. For example, the trait may increase the ability of the plant to acquire water by at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold or more. In some embodiments, the trait confers improved photosynthetic efficiency as compared to a plant that does not comprise the cells described herein. For example, the trait may increase photosynthetic efficiency of the plant by at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold or more.

### Delivery Construct

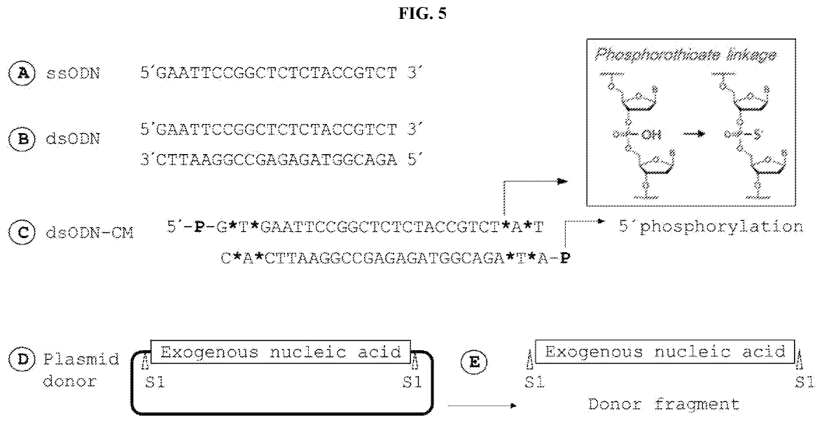

Provided herein, in certain embodiments, is a first element comprising a donor nucleic acid sequence (e.g., donor DNA). As shown in FIG. 5, the donor nucleic acid (e.g., donor DNA) may be A) a blunt single-stranded oligodeoxynucleotide (ssODN), B) a blunt linear double-stranded oligodeoxynucleotide (dsODN), or C) a chemically modified dsODN (dsODN-CM) which is flanked by two additional nucleotides with phosphorothioate linkages (asterisk) at the 5′- and 3′-ends of both DNA strands. The dsODN-CM also contain a phosphorylation (bolded P) at the 5′ end of both strand of the exogenous nucleic acid. D) The donor DNA can also be delivered as a plasmid (plasmid donor), containing two equal sites for nuclease cleavage (S1) within a guide sequence, bearing the exogenous nucleic acid sequence, wherein the guide sequence is the same guide sequence of the non-coding region, intron. E) The plasmid donor cleaved by nuclease at S1 sites, releases the donor fragment of exogenous nucleic acid.

Also provided herein is a second element (e.g., plasmid) comprising a sequence encoding a DNA nuclease. In some embodiments, the DNA nuclease is a CRISPR associated nuclease. In a particular embodiment, the CRISPR associated nuclease comprises Cas9. In some embodiments, the second plasmid encodes one or more guide RNA (gRNA). Non-limiting examples of gRNA are described in Table 4. In other embodiments, the DNA nuclease is a Transcription Activator-Like Effector Nuclease (TALEN). In certain embodiments, the sequence encoding the DNA nuclease is fused to a sequence encoding VirD2 as described in Table 5.

Further provided herein is a kit that comprises the first element (e.g., donor plasmid) described herein and the second element (e.g., plasmid) comprising a sequence encoding a DNA nuclease described herein.

Further provided are combinations comprising the first element and optionally the second element, and a cell for insertion of the donor nucleic acid. In some embodiments, the cell comprises an acceptor non-coding region, for example an intron, for insertion of the donor nucleic acid sequence. In some embodiments, the cell is a plant cell. In some embodiments, the plant is a dicotyledonous plant. In some embodiments, the dicotyledonous plant is selected from Table 9. In some embodiments, the plant is a monocotyledonous plant. In some embodiments, the monocotyledonous plant is selected from Table 9. In some embodiments, the plant cell is a ground tissue cell. In some embodiments, the tissue cell is a parenchyma, collenchyma, or sclerenchyma cell. In some embodiments, the plant cell is a vascular tissue cell. In some embodiments, the tissue cell is a tracheid, vessel element, sieve tube cell, or companion cell. In some embodiments, the plant cell is a dermal tissue cell. In some embodiments, the tissue cell is an epidermal, guard cell, or trichome. In some embodiments, the cell is not transgenic. In some embodiments, the exogenous nucleic acid is introduced into the cell via non-homologous recombination. In some embodiments, the exogenous nucleic acid is introduced into the cell via non-homologous end-joining. In some embodiments, the exogenous nucleic acid is introduced into the cell via homology-independent targeted integration (HITI). In some embodiments, the exogenous nucleic acid is introduced into the cell via nuclease gene editing. In some embodiments, the nuclease gene editing comprises CRISPR-Cas gene editing.

### Methods of Preparing Compositions

Various embodiments provide for methods of generating a cell comprising an exogenous nucleic acid described herein. In some embodiments, the method comprises introducing into a non-coding region, e.g., an intron, 5′ non-coding or 3′ non-coding region, of the cell the endogenous or exogenous nucleic acid.

Provided herein, in some embodiments, are methods of generating a cell comprising an endogenous or exogenous nucleic acid in a non-coding region, e.g., an intron, of the cell. In some embodiments, the method comprises introducing into the cell the donor nucleic acid (e.g., donor DNA) described herein.

Also provided herein, in some embodiments, are methods of generating a host comprising an endogenous or exogenous nucleic acid. In some embodiments, the method comprises introducing into a non-coding region, e.g., intron, of the host the endogenous or exogenous nucleic acid.

Further provided herein, in some embodiments, are methods of generating a host comprising an endogenous or exogenous nucleic acid in a non-coding region, e.g., an intron, of the host. In some embodiments, the method comprises introducing into the host the donor nucleic acid (e.g., donor DNA) described herein.

In various embodiments, insertion of endogenous or the exogenous nucleic acid may be done by non-homologous insertion into a non-coding region, e.g., intron, via nuclease gene editing or any other gene editing method that does not require homologous recombination. Therefore, the modified cells are not transgenic. For example, a precise nuclease mediated integration into the non-coding region, e.g., intron, of the genes may be performed by using the homology-independent targeted integration (HITI), which explores the DNA repair system directed by non-homologous end joining (NHEJ).

In some embodiments, the endogenous or exogenous nucleic acid is introduced via non-homologous recombination. In some embodiments, the endogenous or exogenous nucleic acid is introduced via non-homologous end-joining. In some embodiments, the endogenous or exogenous nucleic acid is introduced via homology-independent targeted integration (HITI). In some embodiments, the endogenous or exogenous nucleic acid is introduced cell via nuclease gene editing. In a particular embodiment, the nuclease gene editing comprises CRISPR-Cas gene editing.

### Methods of Use

Provided herein, in some embodiments, are methods of reducing or eliminating expression of a target gene in a cell. In some embodiments, the method comprises introducing into a non-coding region, e.g., an intron, of the cell an endogenous or exogenous nucleic acid. In certain embodiments, the endogenous or exogenous nucleic acid encodes for a sequence that is capable of binding to mRNA of the target gene, thereby reducing or eliminating expression of the target gene.

Provided herein, in some embodiments, are methods of introducing, increasing, or reducing a trait in a host. In some embodiments, the method comprises introducing into a non-coding region, e.g., an intron, of a cell of the host an endogenous or exogenous nucleic acid. In certain embodiments, the endogenous or exogenous nucleic acid encodes for a sequence that is capable of binding to mRNA of the target gene, thereby introducing, increasing, or reducing a trait in a host.

Provided herein, in some embodiments, are methods of regulating a target gene or peptide in a cell. In some embodiments, the method comprises introducing into a non-coding region, e.g., an intron, of a cell of the host an endogenous or exogenous nucleic acid. In certain embodiments, the endogenous or exogenous nucleic acid encodes for a sequence that is capable of binding to mRNA of the target gene, thereby regulating a target gene or peptide in a cell.

Provided herein, in some embodiments, are methods of introducing, increasing, or reducing a trait in a host. In some embodiments, the method comprises introducing into a non-coding region, e.g., an intron, of a cell of the host an endogenous or exogenous nucleic acid. In certain embodiments, the endogenous or exogenous nucleic acid encodes for a sequence that is capable of binding to mRNA of the target gene, thereby introducing, increasing, or reducing a trait in a host.

### Kits

Further provided is a kit to perform methods described herein. The kit is an assemblage of components, including at least one of the compositions described herein. Thus, in some embodiments, the kit comprises a nucleic acid and/or peptide composition described herein. The nucleic acid or peptide may be combined with, or complexed to, another component, such as a vehicle for delivery, or may be unmodified for direct delivery.

Instructions for use of the components may be included in the kit. Optionally, the kit also contains other useful components, such as, diluents, buffers, pharmaceutically acceptable carriers, syringes, applicators, measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of a composition containing a nucleic acid herein. The packaging material generally has an external label which indicates the contents and/or purpose of the kit and/or its components.

### Certain Definitions

Percent (%) sequence identity with respect to a reference polypeptide or polynucleotide sequence is the percentage of amino acid or nucleotide residues in a candidate sequence that are identical with the amino acid or nucleotide residues in the reference polypeptide or polynucleotide sequence after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences can be determined, including algorithms needed to achieve maximal alignment over the full length of the sequences being compared. For purposes herein, however, % amino acid or polynucleotide sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid or polynucleotide sequence comparisons, the % amino acid or polynucleotide sequence identity of a given sequence A to, with, or against a given sequence B (which can alternatively be phrased as a given sequence A that has or comprises a certain % sequence identity to, with, or against a given sequence B is calculated as follows: 100 times the fraction X/Y, where X is the number of residues scored as identical matches by the sequence alignment program ALIGN-2 in that program's alignment of A and B, and where Y is the total number of residues in B. It will be appreciated that where the length of sequence A is not equal to the length of sequence B, the % sequence identity of A to B will not equal the % sequence identity of B to A. Unless specifically stated otherwise, all % sequence identity values used herein are obtained as described in the immediately preceding paragraph using the ALIGN-2 computer program.

In some embodiments, the term “about” means within 10% of the stated amount. For instance, a peptide comprising about 80% identity to a reference peptide may comprise 72% to 88% identity to the reference peptide sequence.

### NON-LIMITING EXAMPLE EMBODIMENTS

1. A cell comprising a non-coding region, wherein the non-coding region comprises an endogenous or exogenous nucleic acid, optionally, wherein the non-coding region comprises (i) a modified intron region positioned between a first exon region and a second exon region, (ii) a 5′ non-coding region, or (iii) a 3′ non-coding region, or (iv) at least two of (i)-(iii). 2. The cell of embodiment 1, wherein the non-coding region is modified from an intron of a gene. 3. The cell of embodiment 2, wherein the gene is endogenous to the cell. 4. The cell of embodiment 2 or embodiment 3, wherein the endogenous or exogenous nucleic acid is positioned within the non-coding region of the gene, or within a portion of the non-coding region of the gene. 5. The cell of any one of embodiments 2-4, wherein the endogenous or exogenous nucleic acid does not replace any nucleobases of the non-coding region of the gene. 6. The cell of any one of embodiments 2-4, wherein the endogenous or exogenous nucleic acid replaces 1-10, 1-20, 10-30, or 10-40 nucleobases of the non-coding region of the gene. 7. The cell of any one of embodiments 2-6, wherein the non-coding region comprises a first portion of the intron of the gene, the endogenous or exogenous nucleic acid, and a second portion of the intron of the gene. 8. The cell of any one of embodiments 2-7, wherein the intron of the gene is selected from Table 2. 9. The cell of any one of embodiments 2-8, wherein the gene is selected from Table 1. 10. The cell of any one of embodiments 2-9, wherein the gene comprises a plurality of introns. 11. The cell of embodiment 10, wherein the plurality of introns is about 2, 3, 4, 5, 6, 7, 8, 9, 10, 11, 12, 13, 14, 15, 16, 17, 18, 19, 20 introns (e.g., as exemplified by genes from Table 1). 12. The cell of embodiment 11, wherein the non-coding region is present in the first, second, third, fourth, fifth, sixth, seventh, eighth, nineth, tenth, eleventh, twelfth, thirteenth, fourteenth, fifteenth, sixteenth, seventeenth, eighteenth, nineteenth, or twentieth intron of the gene, as applicable. 13. The cell of any one of embodiments 2-12, wherein the first exon region and the second exon region are regions of the gene. 14. The cell of embodiment 1, wherein (i) the non-coding region comprises the modified intron region positioned between the first exon region and the second exon region, and wherein the first exon region and the second exon region are regions of a gene, (ii) the non-coding region comprises the 5′ non-coding region, and the 5′ non-coding region is upstream of a gene, or (iii) the non-coding region comprises the 3′ non-coding region, and the 3′ non-coding region is downstream of a gene. 15. The cell of embodiment 14 or any one of embodiments 303-305, wherein the gene is endogenous to the cell. 16. The cell of any one of embodiments 2-15, wherein the gene is constitutively expressed. 17. The cell of any one of embodiments 2-16, wherein the gene is expressed in a specific tissue or organ. 18. The cell of embodiment 17, wherein the cell is a plant cell, and the tissue or organ comprises a root, stem, fruit, seed, leaf, ground tissue, vascular tissue, or dermal tissue, or a combination of two or more thereof. 19. The cell of any one of embodiments 2-18, wherein the gene is expressed at a range of 1-5%, 1-10%, 5-15%, or 5-20% of the total expressed genes in the cell (e.g., as determined by mRNA expression profiling of the said cell). 20. The cell of any one of embodiments 2-19, wherein upon transcription and mRNA splicing, the native mRNA of the gene is translated into the native protein of the gene. 21. The cell of any one of embodiments 2-20, wherein the gene encodes a native protein. 22. The cell of embodiment 20 or embodiment 21, wherein the native protein is actin, ubiquitin, ribosomal protein, heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, RB7, or any other protein expressed from a gene of Table 1. 23. The cell of any one of embodiments 2-22, wherein the gene is selected from Table 1. 24. The cell of any one of embodiments 2-23, wherein the exogenous nucleic acid is transcribed from a promoter. 25. The cell of embodiment 24, wherein the promoter is a promoter native to the gene. 26. The cell of embodiment 1, wherein the endogenous or exogenous nucleic acid is transcribed from a promoter. 27. The cell of any one of embodiments 24-26, wherein the promoter is a constitutive promoter. 28. The cell of any one of embodiments 24-27, wherein the promoter is specific for a plant organ. 29. The cell of embodiment 28, wherein the plant organ is a root, stem, fruit, seed, or leaf. 30. The cell of any one of embodiments 24-29, wherein the promoter is specific for a plant tissue. 31. The cell of embodiment 30, wherein the plant tissue is a ground tissue, vascular tissue, or dermal tissue. 32. The cell of any one of embodiments 24-31, wherein the promoter is an endogenous promoter of the cell. 33. The cell of any one of embodiments 24-32, wherein the promoter drives the expression of one gene selected from Table 1. 34. The cell of any one of embodiments 1-33, wherein the non-coding region comprises one or more nucleases recognition sites. 35. The cell of embodiment 34, wherein at least one of the one or more nuclease recognition sites is selected from Table 3. 36. The cell of any one of embodiments 1-35, wherein the endogenous or exogenous nucleic acid is about 10 to about 700 bases in length, 10 to about 600 bases in length, 10 to about 500 bases in length, 10 to about 400 bases in length, 10 to about 300 bases in length, 10 to about 200 bases in length, 10 to about 180 bases, about 10 to about 160 bases, about 10 to about 140 bases, about 10 to about 120 bases, about 10 to about 110 bases, or about 10 to about 100 bases in length. 37. The cell of any one of embodiments 1-36, wherein the endogenous or exogenous nucleic acid is less than 200 bases in length. 38. The cell of any one of embodiments 1-37, wherein the endogenous or exogenous nucleic acid is positioned within the genome of the cell. 39. The cell of any one of embodiments 1-38, wherein the endogenous or exogenous nucleic acid is not present on a plasmid. 40. The cell of any one of embodiments 1-39, wherein the endogenous or exogenous nucleic acid encodes a micro RNA (miRNA). 41. The cell of embodiment 40, wherein the miRNA is expressed as a short tandem target mimic (STTM) comprising two copies of partially complementary RNA linked by a spacer. 42. The cell of embodiment 41, wherein the spacer has a length of about 6 to about 60 nucleobases. 43. The cell of embodiment 41 or embodiment 42, wherein each of the two copies of partially complementary RNA have a length of about 10 to about 30 nucleobases. 44. The cell of any one of embodiments 40-43, wherein the miRNA specifically binds to a target nucleic acid. 45. The cell of embodiment 44, wherein the target nucleic acid is exogenous to the cell. 46. The cell of embodiment 44, wherein the target nucleic acid is endogenous to the cell. 47. The cell of any one of embodiments 44-46, wherein the target nucleic acid is responsible for water acquisition, nutrient acquisition, disease control, or pest control, or any combination thereof. 48. The cell of any one of embodiments 44-47, wherein the target nucleic acid comprises a regulatory element involved in: plant growth and development, yield, biotic stress, abiotic stress, or herbicide resistance, or any combination thereof. 49. The cell of any one of embodiments 44-48, wherein the target nucleic acid is from an insect, bacteria, fungi, worm (e.g., larva of the insect, and nematode), or a combination thereof, that is harmful to the cell. 50. The cell of any one of embodiments 44-49, wherein the target nucleic acid is present in a target pest selected from Table 6. 51. The cell of any one of embodiments 44-50, wherein the target nucleic acid is selected from the target genes in Table 6. 52. The cell of any one of embodiments 44-51, wherein the target nucleic acid is from an organism that causes a disease to the cell. 53. The cell of embodiment 52, wherein the organism is any one selected from Table 6. 54. The cell of any one of embodiments 44-53, wherein the target nucleic acid is a target mRNA. 55. The cell of embodiment 54, wherein the target mRNA comprises a sequence at least 70% identical to a sequence of Table 6. 56. The cell of embodiment 54 or embodiment 55, wherein the target mRNA is encoded from a target gene. 57. The cell of embodiment 56, wherein the target gene is selected from a gene of Table 6. 58. The cell of embodiment 56 or embodiment 57, wherein the target gene comprises a sequence at least 70% identical to a sequence of Table 6. 59. The cell of any one of embodiments 1-58, wherein the exogenous nucleic acid comprises a sequence at least 70% identical to a sequence of any one of the target gene sequences of Table 6, or the exogenous nucleic acid comprises a sequence at least 80% identical to at least 10 contiguous bases of any one of the target gene sequences of Table 6. 60. The cell of any one of embodiments 1-59, wherein the exogenous nucleic acid encodes a peptide. 61. The cell of embodiment 60, wherein the coding region for the peptide is flanked by a 5′ribosomal binding site (RBS). 62. The cell of embodiment 61, wherein the RBS is 4-80 bases in length. 63. The cell of any one of embodiments 60-62, wherein the peptide affects one or more property of the cell selected from: hormonal regulation, protection against a pathogen, protection against an insect, nitrogen fixation, nutrient acquisition, immunity induction, biotic stress, or abiotic stress, or a combination thereof. 64. The cell of any one of embodiments 60-63, wherein the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. 65. The cell of any one of embodiments 60-64, wherein the peptide is selected from Table 7. 66. The cell of any one of embodiments 60-65, wherein the peptide is encoded by a sequence at least 80% identical to a sequence of Table 8. 67. The cell of any one of embodiments 1-66, wherein the exogenous nucleic acid comprises a sequence at least 80% identical to a sequence of Table 8.

68. A cell comprising an endogenous or exogenous micro RNA (miRNA). 69. The cell of embodiment 68, wherein the exogenous miRNA is an artificial micro RNA (amiRNA). 70. The cell of embodiment 68 or embodiment 69, wherein the endogenous or exogenous miRNA is expressed as a short tandem target mimic (STTM) comprising two copies of partially complementary RNA linked by a spacer. 71. The cell of embodiment 70, wherein the spacer has a length of about 6 to about 60 nucleobases. 72. The cell of embodiment 70 or embodiment 71, wherein each of the two copies of partially complementary RNA have a length of about 10 to about 30 nucleobases. 73. The cell of any one of embodiments 68-72, wherein the endogenous or exogenous miRNA is a precursor miRNA. 74. The cell of any one of embodiments 68-72, wherein the endogenous or exogenous miRNA is a mature miRNA. 75. The cell of embodiment 74, wherein the mature miRNA comprises about 21-22 nucleotides. 76. The cell of any one of embodiments 68-75, wherein the miRNA specifically binds to a target nucleic acid. 77. The cell of embodiment 76, wherein the target nucleic acid is exogenous to the cell. 78. The cell of embodiment 76, wherein the target nucleic acid is endogenous to the cell. 79. The cell of any one of embodiments 76-78, wherein the target nucleic acid is responsible for water acquisition, nutrient acquisition, disease control, or pest control, or any combination thereof. 80. The cell of any one of embodiments 76-79, wherein the target nucleic acid comprises a regulatory element involved in: plant growth and development, yield, biotic stress, abiotic stress, or herbicide resistance, or any combination thereof. 81. The cell of any one of embodiments 76-80, wherein the target nucleic acid is from an insect, bacteria, fungi, nematode or a worm, or a combination thereof, that is harmful to the cell. 82. The cell of any one of embodiments 76-81, wherein the target nucleic acid is present in a target pest selected from Table 6. 83. The cell of any one of embodiments 76-82, wherein the target nucleic acid is selected from the target genes in Table 6. 84. The cell of any one of embodiments 76-83, wherein the target nucleic acid is from an organism that causes a disease to the cell. 85. The cell of embodiment 84, wherein the organism is any one selected from Table 6. 86. The cell of any one of embodiments 76-85, wherein the target nucleic acid is a target mRNA. 87. The cell of embodiment 86, wherein the target mRNA comprises a sequence at least 70% identical to a sequence of Table 6. 88. The cell of embodiment 86 or embodiment 87, wherein the target mRNA is encoded from a target gene. 89. The cell of embodiment 88, wherein the target gene is selected from a gene of Table 6. 90. The cell of embodiment 88 or embodiment 89, wherein the target gene comprises a sequence at least 70% identical to a sequence of Table 6.

91. A cell comprising an endogenous or exogenous mRNA encoding a peptide. 92. The cell of embodiment 91, wherein the endogenous or exogenous mRNA is flanked by a 5′ribosomal binding site (RBS). 93. The cell of embodiment 92, wherein the RBS is 4-20 base pair in length. 94. The cell of any one of embodiments 91-93, wherein the peptide affects one or more property of the cell selected from: hormonal regulation, protection against a pathogen, protection against an insect, nitrogen fixation, nutrient acquisition, immunity induction, biotic stress, or abiotic stress, or a combination thereof. 95. The cell of any one of embodiments 91-94, wherein the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. 96. The cell of any one of embodiments 91-95, wherein the peptide is selected from Table 7. 97. The cell of any one of embodiments 91-96, wherein the peptide is encoded by a sequence at least 80% identical to a sequence of Table 8. 98. The cell of any one of embodiments 91-97, wherein the mRNA comprises a sequence at least 80% identical to a sequence of Table 8.

99. A cell comprising an endogenous or exogenous peptide. 100. The cell of embodiment 99, wherein the peptide affects one or more property of the cell selected from: hormonal regulation, protection against a pathogen, protection against an insect, nitrogen fixation, nutrient acquisition, immunity induction, biotic stress, or abiotic stress, or a combination thereof. 101. The cell of embodiment 99 or embodiment 100, wherein the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. 102. The cell of any one of embodiments 99-101, wherein the peptide is selected from Table 7. 103. The cell of any one of embodiments 99-102, wherein the peptide is encoded by a sequence at least 80% identical to a sequence of Table 8. 104. The cell of any one of embodiments 1-103, wherein the cell is a plant cell. 105. The cell of embodiment 104, wherein the plant is a dicotyledonous plant. 106. The cell of embodiment 105, wherein the dicotyledonous plant is selected from Table 9. 107. The cell of embodiment 104, wherein the plant is a monocotyledonous plant. 108. The cell of embodiment 107, wherein the monocotyledonous plant is selected from Table 9. 109. The cell of any one of embodiments 104-108, wherein the plant cell is a ground tissue cell. 110. The cell of embodiment 109, wherein the tissue cell is a parenchyma, collenchyma, or sclerenchyma cell. 111. The cell of any one of embodiments 104-108, wherein the plant cell is a vascular tissue cell. 112. The cell of embodiment 111, wherein the tissue cell is a tracheid, vessel element, sieve tube cell, or companion cell. 113. The cell of any one of embodiments 104-108, wherein the plant cell is a dermal tissue cell. 114. The cell of embodiment 113, wherein the tissue cell is a epidermal, guard cell, or trichome.

115. The cell of any one of embodiments 1-114, wherein the cell is not transgenic. 116. The cell of any one of embodiments 1-67 or embodiments 104-115, wherein the endogenous or exogenous nucleic acid is introduced into the cell via non-homologous recombination. 117. The cell of embodiment 116, wherein the endogenous or exogenous nucleic acid is introduced into the cell via non-homologous end-joining. 118. The cell of embodiment 116 or embodiment 117, wherein the endogenous or exogenous nucleic acid is introduced into the cell via homology-independent targeted integration (HITI). 119. The cell of any one of embodiments 1-67 or embodiments 104-118, wherein the endogenous or exogenous nucleic acid is introduced into the cell via nuclease gene editing. 120. The cell of embodiment 119, wherein the nuclease gene editing comprises CRISPR-Cas gene editing.

121. A host comprising the cell of any one of embodiments 1-120. 122. The host of embodiment 121, wherein the host is a plant. 123. The host of embodiment 122, wherein the plant is a dicotyledonous plant. 124. The host of embodiment 123, wherein the dicotyledonous plant is selected from Table 9. 125. The host of embodiment 122, wherein the plant is a monocotyledonous plant. 126. The host of embodiment 125, wherein the monocotyledonous plant is selected from Table 9. 127. The host of any one of embodiments 122-126, wherein the plant is not transgenic.

128. A seed from the plant of any one of embodiments 122-127.

129. A plant obtained from the seed of embodiment 128. 130. The plant of any one of embodiments 122-127 or embodiment 129, wherein the plant has one or more traits. 131. The plant of embodiment 130, wherein the one or more traits comprises hormonal regulation, protection against a pathogen, protection against an insect, nitrogen fixation, nutrient acquisition, immunity induction, biotic stress, or abiotic stress, or a combination thereof. 132. The plant of embodiment 130 or embodiment 131, wherein the trait is conferred by an endogenous or exogenous nucleic acid and/or peptide. 133. The plant of embodiment 132, wherein the endogenous or exogenous nucleic acid and/or peptide provides hormonal regulation, protection against a pathogen, protection against an insect, nitrogen fixation, nutrient acquisition, immunity induction, biotic stress, or abiotic stress, or a combination thereof. 134. The plant of any one of embodiments 130-133, wherein the trait comprises resistance to a pest. 135. The plant of embodiment 134, wherein the pest is an insect, bacteria, fungi, worm (e.g., larva of the insect, and nematode), or a combination thereof. 136. The plant of embodiment 134 or embodiment 135, wherein the pest is selected from Table 6. 137. The plant of any one of embodiments 134-136, wherein the resistance is due to antibiosis (growth and multiplication of the pest is inhibited), antixenosis (the pest is repelled by the plant), or tolerance (plant is able to withstand or recover from damage by the pest). 138. The plant of any one of embodiments 134-137, wherein the resistant plant has a superior yield as compared to a plant that does not comprise the cell of any one of embodiments 1-120, when the plants are both under attack by the pest. 139. The plant of any one of embodiments 130-138, wherein the trait comprises resistance to a disease. 140. The plant of embodiment 139, wherein the disease is caused by a pest. 141. The plant of embodiment 140, wherein the pest is an insect, bacteria, fungi, worm (e.g., larva of the insect, and nematode), or a combination thereof. 142. The plant of embodiment 140 or embodiment 141, wherein the pest is selected from Table 6. 143. The plant of any one of embodiments 139-142, wherein the resistance is due to antibiosis (growth and multiplication of the pest is inhibited), antixenosis (the pest is repelled by the plant), or tolerance (plant is able to withstand or recover from damage by the pest). 144. The plant of any one of embodiments 139-143, wherein the resistant plant has a superior yield as compared to a plant that does not comprise the cell of any one of embodiments 1-120, when the plants are both exposed to the disease. 145. The plant of any one of embodiments 130-144, wherein the trait comprises resistance to a chemical. 146. The plant of embodiment 145, wherein the chemical is a weed control chemical. 147. The plant of embodiment 145, wherein the weed control chemical is a growth inhibitor. 148. The plant of embodiment 145, wherein the chemical is a herbicide. 149. The plant of embodiment 148, wherein the herbicide is 2,4-D (2,4-dichlorophenoxy acetic acid), Aminopyralid, Atrazine, Clopyralid, Dicamba, Glufosinate ammonium, Fluazifop, Fluroxypyr, Glyphosate, Imazapyr, Imazapic, Imazamox, Linuron, MCPA (2-methyl-4-chlorophenoxyacetic acid), Metolachlor, Paraquat, Pendimethalin, Picloram, Sodium chlorate, Triclopyr, Sulfonylureas (e.g., Flazasulfuron and Metsulfuron-methyl), or a combination thereof. 150. The plant of any one of embodiments 130-149, wherein the trait confers an improved nutritional and/or visual quality as compared to a plant that does not comprise the cell of any one of embodiments 1-120, (e.g., measurable using a spectrometric method). 151. The plant of any one of embodiments 130-150, wherein the trait confers an increase in crop yield as compared to a plant that does not comprise the cell of any one of embodiments 1-120. 152. The plant of any one of embodiments 130-151, wherein the trait confers an ability to acquire a nutrient (e.g., nitrogen, phosphorus, potassium and/or plant micronutrients) at least 10% more efficiently as compared to a plant that does not comprise the cell of any one of embodiments 1-120 (e.g., measurable using a spectrophotometric method). 153. The plant of any one of embodiments 130-152, wherein the trait confers an ability to acquire water at least 10% more efficiently as compared to a plant that does not comprise the cell of any one of embodiments 1-120 (e.g., measurable using the plant fresh weight when they were subjected to, for example, drought stress). 154. The plant of any one of embodiments 130-153, wherein the trait confers at least 10% improved photosynthetic efficiency as compared to a plant that does not comprise the cell of any one of embodiments 1-120 (e.g., measurable using, for example, a gas-exchange analyzer).

155. A donor nucleic acid sequence comprising an endogenous or exogenous nucleic acid. 156. The donor nucleic acid of embodiment 155, wherein the endogenous or exogenous nucleic acid is about 10 to about 700 bases in length, about 10 to about 600 bases in length, about 10 to about 500 bases in length, about 10 to about 400 bases in length, about 10 to about 300 bases in length, about 10 to about 200 bases in length, about 10 to about 180 bases, about 10 to about 160 bases, about 10 to about 140 bases, about 10 to about 120 bases, about 10 to about 110 bases, or about 10 to about 100 bases in length. 157. The donor nucleic acid of embodiment 155 or embodiment 156, wherein the endogenous or exogenous nucleic acid is less than 200 bases in length. 158. The donor nucleic acid of any one of embodiments 155-157, wherein the endogenous or exogenous nucleic acid encodes a micro RNA (miRNA). 159. The donor nucleic acid of embodiment 158, wherein the miRNA is expressed as a short tandem target mimic (STTM) comprising two copies of partially complementary RNA linked by a spacer. 160. The donor nucleic acid of embodiment 159, wherein the spacer has a length of about 6 to about 60 nucleobases. 161. The donor nucleic acid of embodiment 159 or embodiment 160, wherein each of the two copies of partially complementary RNA have a length of about 10 to about 30 nucleobases. 162. The donor nucleic acid of any one of embodiments 158-161, wherein the miRNA specifically binds to a target nucleic acid. 163. The donor nucleic acid of embodiment 162, wherein the target nucleic acid is responsible for water acquisition, nutrient acquisition, disease control, or pest control, or any combination thereof. 164. The donor nucleic acid of embodiment 162 or embodiment 163, wherein the target nucleic acid comprises a regulatory element involved in: plant growth and development, yield, biotic stress, abiotic stress, or herbicide resistance, or any combination thereof. 165. The donor nucleic acid of any one of embodiments 162-164, wherein the target nucleic acid is from an insect, bacteria, fungi, worm (e.g., larva of the insect, and nematode), or a combination thereof, that is harmful to a cell. 166. The donor nucleic acid of any one of embodiments 162-165, wherein the target nucleic acid is present in a target pest selected from Table 6. 167. The donor nucleic acid of any one of embodiments 162-166, wherein the target nucleic acid is selected from the target genes in Table 6. 168. The donor nucleic acid of any one of embodiments 162-167, wherein the target nucleic acid is from an organism that causes a disease to a cell. 169. The donor nucleic acid of embodiment 168, wherein the organism is any one selected from Table 6. 170. The donor nucleic acid of any one of embodiments 162-169, wherein the target nucleic acid is a target mRNA. 171. The donor nucleic acid of embodiment 170, wherein the target mRNA comprises a sequence at least 70% identical to a sequence of Table 6. 172. The donor nucleic acid of embodiment 170 or embodiment 171, wherein the target mRNA is encoded from a target gene. 173. The donor nucleic acid of embodiment 172, wherein the target gene is selected from a gene of Table 6. 174. The donor nucleic acid of embodiment 172 or embodiment 173, wherein the target gene comprises a sequence at least 70% identical to a sequence of Table 6. 175. The donor nucleic acid of any one of embodiments 155-174, wherein the endogenous or exogenous nucleic acid comprises a sequence at least 70% identical to a sequence of any one of the target gene sequences of Table 6, or the exogenous nucleic acid comprises a sequence at least 80% identical to at least 10 contiguous bases of any one of the target gene sequences of Table 6. 176. The donor nucleic acid of any one of embodiments 155-157, wherein the endogenous or exogenous nucleic acid encodes a peptide. 177. The donor nucleic acid of embodiment 176, wherein the coding region for the peptide is flanked by a 5′ribosomal binding site (RBS). 178. The donor nucleic acid of embodiment 177, wherein the RBS is 4-20 bases in length. 179. The donor nucleic acid of any one of embodiments 176-178, wherein the peptide affects one or more property of a cell selected from: hormonal regulation, protection against a pathogen, protection against an insect, nitrogen fixation, nutrient acquisition, immunity induction, biotic stress, or abiotic stress, or a combination thereof. 180. The donor nucleic acid of any one of embodiments 176-179, wherein the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. 181. The donor nucleic acid of any one of embodiments 176-180, wherein the peptide is selected from Table 7. 182. The donor nucleic acid of any one of embodiments 176-181, wherein the peptide is encoded by a sequence at least 80% identical to a sequence of Table 8. 183. The donor nucleic acid of any one of embodiments 155-182, wherein the endogenous or exogenous nucleic acid comprises a sequence at least 80% identical to a sequence of Table 8. 184. The donor nucleic acid of any one of embodiments 155-183, wherein the donor nucleic acid is a blunt linear double-stranded oligodeoxynucleotide (dsODN). 185. The donor nucleic acid of any one of embodiments 155-183, wherein the donor nucleic acid is a single-stranded oligodeoxynucleotide (ssODN). 186. The donor nucleic acid of any one of embodiments 155-183, wherein the donor nucleic acid is a plasmid donor. 187. The donor of nucleic acid of any one of embodiments 155-186, comprising one or two nuclease recognition sites. 188. The donor nucleic acid of any one of embodiments 155-187, comprising 2 nucleotides of phosphorothioate linkages at the 5′- and 3′-ends of both DNA strands of the exogenous nucleic acid. 189. The donor nucleic acid of any one of embodiments 155-188, wherein the donor nucleic acid is phosphorylated at the 5′ end of both strands of the exogenous nucleic acid.

190. A kit comprising the donor nucleic acid of any one of embodiments 155-189, and a nucleic acid sequence encoding a DNA nuclease. 191. The kit of embodiment 190, wherein the DNA nuclease is as exemplified in Example 1. 192. The kit of embodiment 190 or embodiment 191, wherein the DNA nuclease is a CRISPR associated nuclease. 193. The kit of embodiment 192, wherein the CRISPR associated nuclease comprises Cas9. 194. The kit of any one of embodiments 190-193, wherein the nucleic acid sequence encoding the DNA nuclease further encodes one or more guide RNA (gRNA). 195. The kit of embodiment 194, wherein the one or more gRNA are selected from Table 4. 196. The kit of embodiment 190, wherein the DNA nuclease is a Transcription Activator-Like Effector Nuclease (TALEN). 197. The kit of any one of embodiments 190-196, wherein the DNA nuclease is connected to a sequence encoding VirD2 (e.g., Table 5).

198. A combination comprising the donor nucleic acid of any one of embodiments 155-189, or the kit of any one of embodiments 190-197, and a cell comprising an acceptor non-coding region for insertion of the donor nucleic acid sequence. 199. The combination of embodiment 198, wherein the cell is a plant cell. 200. The combination of embodiment 199, wherein the plant is a dicotyledonous plant. 201. The combination of embodiment 200, wherein the dicotyledonous plant is selected from Table 9. 202. The combination of embodiment 199, wherein the plant is a monocotyledonous plant. 203. The combination of embodiment 202, wherein the monocotyledonous plant is selected from Table 9. 204. The combination of embodiment 199, wherein the plant cell is a ground tissue cell. 205. The combination of embodiment 204, wherein the tissue cell is a parenchyma, collenchyma, or sclerenchyma cell. 206. The combination of embodiment 199, wherein the plant cell is a vascular tissue cell. 207. The combination of embodiment 206, wherein the tissue cell is a tracheid, vessel element, sieve tube cell, or companion cell. 208. The combination of embodiment 199, wherein the plant cell is a dermal tissue cell. 209. The combination of embodiment 208, wherein the tissue cell is a epidermal, guard cell, or trichome. 210. The combination of any one of embodiments 198-209, wherein the cell is not transgenic. 211. The combination of any one of embodiments 198-210, wherein the endogenous or exogenous nucleic acid is introduced into the cell via non-homologous recombination. 212. The combination of embodiment 211, wherein the endogenous or exogenous nucleic acid is introduced into the cell via non-homologous end-joining. 213. The combination of embodiment 211 or embodiment 212, wherein the endogenous or exogenous nucleic acid is introduced into the cell via homology-independent targeted integration (HITI). 214. The combination of any one of embodiments 198-213, wherein the endogenous or exogenous nucleic acid is introduced into the cell via nuclease gene editing. 215. The combination of embodiment 214, wherein the nuclease gene editing comprises CRISPR-Cas gene editing.

216. A method of generating a cell with a modified non-coding region, the method comprising introducing into the cell the donor nucleic acid of any one of embodiments 155-189, or the kit of any one of embodiments 190-197. 217. The method of embodiment 216, wherein the modified non-coding region comprises the endogenous or exogenous nucleic acid. 218. A method of generating a cell comprising a modified non-coding region, the method comprising introducing an endogenous or exogenous nucleic acid into a non-coding region of a gene in the cell. 219. The method of any one of embodiments 216-218, wherein the cell is a plant cell. 220. The method of any one of embodiments 216-219, wherein the endogenous or exogenous nucleic acid is introduced via non-homologous recombination. 221. The method of embodiment 220, wherein the endogenous or exogenous nucleic acid is introduced via non-homologous end-joining. 222. The method of embodiment 220 or embodiment 221, wherein the endogenous or exogenous nucleic acid is introduced via homology-independent targeted integration (HITI). 223. The method of any one of embodiments 216-222, wherein the endogenous or exogenous nucleic acid is introduced via nuclease gene editing. 224. The method of embodiment 223, wherein the nuclease gene editing comprises CRISPR-Cas gene editing. 225. A method of reducing or eliminating expression of a target gene in a cell, the method comprising introducing into a non-coding region of the cell an endogenous or exogenous nucleic acid, wherein the endogenous or exogenous nucleic acid encodes for a sequence that is capable of binding to mRNA of the target gene, thereby reducing or eliminating expression of the target gene. 226. A method of regulating a target gene or peptide in a cell, the method comprising introducing into a non-coding region of the cell an endogenous or exogenous nucleic acid, wherein the exogenous nucleic acid encodes for an amino acid sequence that is capable of regulating the target gene or peptide in the cell, thereby regulating the target gene or peptide in the cell. 227. A method of introducing, increasing, or reducing a trait in a host, the method comprising introducing into a non-coding region of a cell of the host an endogenous or exogenous nucleic acid, wherein the endogenous or exogenous nucleic acid encodes for a sequence that is capable of binding to mRNA of a target gene, thereby introducing, increasing, or reducing a trait in the host. 228. A method of introducing, increasing, or reducing a trait in a host, the method comprising introducing into a non-coding region of a cell of the host an exogenous nucleic acid, wherein the endogenous or exogenous nucleic acid encodes for an amino acid sequence that is capable of regulating a target gene or peptide in the cell, thereby introducing, increasing or reducing a trait in the host. 229. The method of embodiment 227 or embodiment 228, wherein the host is a plant. 230. The method of embodiment 229, wherein the plant is a dicotyledonous plant. 231. The method of embodiment 230, wherein the dicotyledonous plant is selected from Table 9. 232. The method of embodiment 229, wherein the plant is a monocotyledonous plant. 233. The method of embodiment 232, wherein the monocotyledonous plant is selected from Table 9. 234. The method of any one of embodiments 229-233, wherein the plant is not transgenic. 235. The method of any one of embodiments 227-234, wherein the trait comprises hormonal regulation, protection against a pathogen, protection against an insect, nitrogen fixation, nutrient acquisition, immunity induction, biotic stress, or abiotic stress, or a combination thereof. 236. The method of any one of embodiments 227-235, wherein the trait comprises resistance to a pest. 237. The method of embodiment 236, wherein the pest is an insect, bacteria, fungi, worm (e.g., larva of the insect, and nematode), or a combination thereof. 238. The method of embodiment 236 or embodiment 237, wherein the pest is selected from Table 6. 239. The method of any one of embodiments 236-238, wherein the resistance is due to antibiosis (growth and multiplication of the pest is inhibited), antixenosis (the pest is repelled by the plant), or tolerance (plant is able to withstand or recover from damage by the pest). 240. The method of any one of embodiments 236-239, wherein the host has a superior yield as compared to a host that does not comprise the exogenous nucleic acid, when the hosts are both under attack by the pest. 241. The method of any one of embodiments 227-240, wherein the trait comprises resistance to a disease. 242. The method of embodiment 241, wherein the disease is caused by a pest. 243. The method of embodiment 242, wherein the pest is an insect, bacteria, fungi, worm (e.g., larva of the insect, and nematode), or a combination thereof. 244. The method of embodiment 242 or embodiment 243, wherein the pest is selected from Table 6. 245. The method of any one of embodiments 241-244, wherein the resistance is due to antibiosis (growth and multiplication of the pest is inhibited), antixenosis (the pest is repelled by the plant), or tolerance (plant is able to withstand or recover from damage by the pest). 246. The method of any one of embodiments 241-245, wherein the resistant host has a superior yield as compared to a host that does not comprise the cell of any one of embodiments 1-120, when the hosts are both exposed to the disease. 247. The method of any one of embodiments 227-246, wherein the trait comprises resistance to a chemical. 248. The method of embodiment 247, wherein the chemical is a weed control chemical. 249. The method of embodiment 248, wherein the weed control chemical is a growth inhibitor. 250. The method of embodiment 247, wherein the chemical is a herbicide. 251. The method of embodiment 250, wherein the herbicide is 2,4-D (2,4-dichlorophenoxy acetic acid), Aminopyralid, Atrazine, Clopyralid, Dicamba, Glufosinate ammonium, Fluazifop, Fluroxypyr, Glyphosate, Imazapyr, Imazapic, Imazamox, Linuron, MCPA (2-methyl-4-chlorophenoxyacetic acid), Metolachlor, Paraquat, Pendimethalin, Picloram, Sodium chlorate, Triclopyr, Sulfonylureas (e.g., Flazasulfuron and Metsulfuron-methyl), or a combination thereof. 252. The method of any one of embodiments 227-251, wherein the trait confers an improved nutritional and/or visual quality as compared to a host that does not comprise the exogenous nucleic acid (e.g., measurable using a spectrometric method). 253. The method of any one of embodiments 227-252, wherein the trait confers an increase in crop yield as compared to a plant that does not comprise the exogenous nucleic acid. 254. The method of any one of embodiments 227-253, wherein the trait confers an ability to acquire a nutrient (e.g., nitrogen, phosphorus, potassium and/or plant micronutrients) at least 10% more efficiently as compared to a host that does not comprise the endogenous or exogenous nucleic acid (e.g., measurable using a spectrophotometric method). 255. The method of any one of embodiments 227-254, wherein the trait confers an ability to acquire water at least 10% more efficiently as compared to a host that does not comprise the endogenous or exogenous nucleic acid (e.g., measurable using the host fresh weight when they were subjected to, for example, drought stress). 256. The method of any one of embodiments 227-255, wherein the trait confers at least 10% improved photosynthetic efficiency as compared to a host that does not comprise the exogenous nucleic acid (e.g., measurable using, for example, a gas-exchange analyzer). 257. The method of any one of embodiments 225-256, wherein the endogenous or exogenous nucleic acid is about 10 to about 700 bases in length, about 10 to about 600 bases in length, about 10 to about 500 bases in length, about 10 to about 400 bases in length, about 10 to about 300 bases in length, about 10 to about 200 bases in length, about 10 to about 180 bases, about 10 to about 160 bases, about 10 to about 140 bases, about 10 to about 120 bases, about 10 to about 110 bases, or about 10 to about 100 bases in length. 258. The method of any one of embodiments 225-257, wherein the endogenous or exogenous nucleic acid is less than 200 bases in length. 259. The method of any one of embodiments 225-258, wherein the endogenous or exogenous nucleic acid encodes a micro RNA (miRNA). 260. The method of embodiment 259, wherein the miRNA is expressed as a short tandem target mimic (STTM) comprising two copies of partially complementary RNA linked by a spacer. 261. The method of embodiment 260, wherein the spacer has a length of about 6 to about 60 nucleobases. 262. The method of embodiment 260 or embodiment 261, wherein each of the two copies of partially complementary RNA have a length of about 10 to about 30 nucleobases. 263. The method of any one of embodiments 259-262, wherein the miRNA specifically binds to a target nucleic acid. 264. The method of embodiment 263, wherein the target nucleic acid is responsible for water acquisition, nutrient acquisition, disease control, or pest control, or any combination thereof. 265. The method of embodiment 263 or embodiment 264, wherein the target nucleic acid comprises a regulatory element involved in: plant growth and development, yield, biotic stress, abiotic stress, or herbicide resistance, or any combination thereof. 266. The method of any one of embodiments 263-265, wherein the target nucleic acid is from an insect, bacteria, fungi, worm (e.g., larva of the insect, and nematode), or a combination thereof, that is harmful to a cell. 267. The method of any one of embodiments 263-266, wherein the target nucleic acid is present in a target pest selected from Table 6. 268. The method of any one of embodiments 263-267, wherein the target nucleic acid is selected from the target genes in Table 6. 269. The method of any one of embodiments 263-268, wherein the target nucleic acid is from an organism that causes a disease to a cell. 270. The method of embodiment 269, wherein the organism is any one selected from Table 6. 271. The method of any one of embodiments 263-270, wherein the target nucleic acid is a target mRNA. 272. The method of embodiment 271, wherein the target mRNA comprises a sequence at least 70% identical to a sequence of Table 6. 273. The method of embodiment 271 or embodiment 272, wherein the target mRNA is encoded from a target gene. 274. The method of embodiment 273, wherein the target gene is selected from a gene of Table 6. 275. The method of embodiment 273 or embodiment 274, wherein the target gene comprises a sequence at least 70% identical to a sequence of Table 6. 276. The method of any one of embodiments 225-275, wherein the endogenous or exogenous nucleic acid comprises a sequence at least 70% identical to a sequence of any one of the target gene sequences of Table 6, or the exogenous nucleic acid comprises a sequence at least 80% identical to at least 10 contiguous bases of any one of the target gene sequences of Table 6. 277. The method of any one of embodiments 225-258, wherein the endogenous or exogenous nucleic acid encodes a peptide. 278. The method of embodiment 277, wherein the endogenous or exogenous nucleic acid is flanked by a 5′ribosomal binding site (RBS). 279. The method of embodiment 278, wherein the RBS is 4-20 bases in length. 280. The method of any one of embodiments 277-279, wherein the peptide affects one or more property of a cell selected from: hormonal regulation, protection against a pathogen, protection against an insect, nitrogen fixation, nutrient acquisition, immunity induction, biotic stress, or abiotic stress, or a combination thereof. 281. The method of any one of embodiments 277-280, wherein the peptide is 2-80 amino acids in length, 3-80, 4-80, 5-80, 6-80, 7-80, 8-80, 9-80, 10-80, 20-80, 30-80, 40-80, 50-80, 60-80, 70-80 or 1-80 amino acids in length. 282. The method of any one of embodiments 277-281, wherein the peptide is selected from Table 7. 283. The method of any one of embodiments 277-282, wherein the peptide is encoded by a sequence at least 80% identical to a sequence of Table 8. 284. The method of any one of embodiments 225-283, wherein the exogenous nucleic acid comprises a sequence at least 80% identical to a sequence of Table 8. 285. The method of any one of embodiments 225-284, wherein the cell is a plant cell. 286. The method of embodiment 285, wherein the plant is a dicotyledonous plant. 287. The method of embodiment 286, wherein the dicotyledonous plant is selected from Table 9. 288. The method of embodiment 285, wherein the plant is a monocotyledonous plant. 289. The method of embodiment 288, wherein the monocotyledonous plant is selected from Table 9. 290. The method of any one of embodiments 285-289, wherein the plant cell is a ground tissue cell. 291. The method of embodiment 290, wherein the tissue cell is a parenchyma, collenchyma, or sclerenchyma cell. 292. The method of any one of embodiments 285-289, wherein the plant cell is a vascular tissue cell. 293. The method of embodiment 292, wherein the tissue cell is a tracheid, vessel element, sieve tube cell, or companion cell. 294. The method of any one of embodiments 285-289, wherein the plant cell is a dermal tissue cell. 295. The method of embodiment 294, wherein the tissue cell is a epidermal, guard cell, or trichome.

296. The method of any one of embodiments 225-295, wherein the cell is not transgenic. 297. The method of any one of embodiments 216-296, wherein the non-coding region comprises an intron and the intron comprises the endogenous or exogeneous nucleic acid. 298. The method of any one of embodiments 216-297, wherein the non-coding region comprises a 5′ non-coding region, and the 5′ non-coding region comprises the endogenous or exogenous nucleic acid. 299. The method of any one of embodiments 216-298, wherein the non-coding region comprises a 3′ non-coding region, and the 3′ non-coding region comprises the endogenous or exogeneous nucleic acid. 300. The donor nucleic acid of any one of embodiments 155-189, the kit of any one of embodiments 190-197, or the combination of any one of embodiments 198-215, wherein the non-coding region comprises an intron and the intron comprises the exogenous nucleic acid. 301. The donor nucleic acid of any one of embodiments 155-189, the kit of any one of embodiments 190-197, or the combination of any one of embodiments 198-215, wherein the non-coding region comprises a 5′ non-coding region, and the 5′ non-coding region comprises the endogenous or exogenous nucleic acid. 302. The donor nucleic acid of any one of embodiments 155-189, the kit of any one of embodiments 190-197, or the method of any one of embodiments 198-215, wherein the non-coding region comprises a 3′ non-coding region, and the 3′ non-coding region comprises the endogenous or exogeneous nucleic acid. 303. The cell of embodiment 14, wherein the non-coding region comprises the modified intron region positioned between the first exon region and the second exon region, and wherein the first exon region and the second exon region are regions of a gene. 304. The cell of embodiment 14, wherein the non-coding region comprises the 5′ non-coding region, and the 5′ non-coding region is upstream of a gene. 305. The cell of embodiment 14, wherein the non-coding region comprises the 3′ non-coding region, and the 3′ non-coding region is downstream of a gene.

### EXAMPLES

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Preparation of a Donor DNA Plasmid and a CRISPR Plasmid

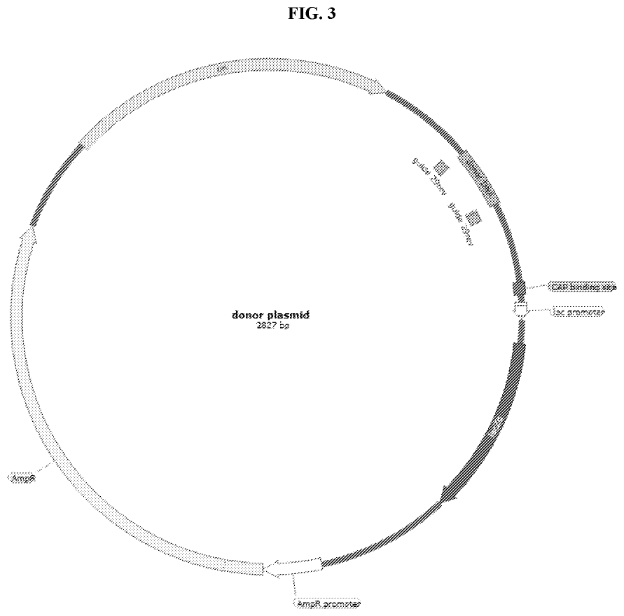

This example illustrates the construction of vectors designed for generating engineered cells described herein. A donor plasmid as described in Table 10 is prepared to deliver the amiRNA. The exemplified amiRNA is the ath-MIR172b (SEQ ID NO: 1471). The amiRNA exemplified is flanked by the guide sequence 29rev from Os03t0718100-01 intron 1 of Table 4, in both sites (5′ and 3′ ends). The two guide sequences and PAM motif enable donor DNA release from the plasmid and insertion on the intron1 of the Actin1 (SEQ ID NO: 1286) in the rice host plant. The original plasmid is the pUC19. A schematic map of the donor plasmid is shown in FIG. 3.

TABLE 10
Donor Plasmid Sequences (from 5′ to 3′). The first column (SEQ ID NO)
contains the sequence identifier of non-limiting examples of acid nucleic sequences of a donor
plasmid. The second column (Feature/Position) describes the feature name and the position of
the sequence into the plasmid. The third column (Sequence) contains the acid nucleic sequence
of the referred feature.

| SEQ | | |
|---|---|---|
| ID | | |
| NO. | Feature/Position | Sequence |
| 1467 | ori | gagatacctacagcgtgagctatgagaaagcgccacgcttcccgaagggagaaaggcggacaggt |
| | (position 1-217) | atccggtaagcggcagggtcggaacaggagagcgcacgagggagcttccagggggaaacgcct |
| | | ggtatctttatagtcctgtcgggtttcgccacctctgacttgagcgtcgatttttgtgatgctcgtc |
| | | aggggggcggagcctatggaaa |
| 1468 | space | aacgccagcaacgcggcctttttacggttcctggccttttgctggccttttgctcacatgttctttc |
| | (position 218- | ctgcgttatcccctgattctgtggataaccgtattaccgcctttgagtgagctgataccgctcgccg |
| | 375) | cagccgaacgaccgagcgcagcga |
| 1469 | guide 29rev | aaatgcagcatttcggtaaa |
| | (position 376- | |
| | 395) | |
| 1470 | PAM | cgg |
| | (position 396- | |
| | 398) | |
| 1471 | donor DNA | AAACGGAGGCGCAGCACCATTAAGATTCACATGGAAATTGA |
| | (position 392- | TAAATACCCTAAATTAGGGTTTTGATATGTATATGAGAATCT |
| | 499) | TGATGATGCTGCATCAACCCGTTT |
| 1472 | PAM | ccg |
| | (position 494- | |
| | 496) | |
| 1473 | Sequence | tttaccgaaatgctgcattt |
| | complementary to | |
| | guide 29rev | |
| | (position 497- | |
| | 516) | |
| 1474 | space | gtcagtgagcgaggaagcggaagagcgcccaatacgcaaaccgcctctccccgcgcgttggccga |
| | (position 517- | ttcattaatgcagctggcacgacaggtttcccgactggaaagcgggcagtgagcgcaacgcaat |
| | 645) | |
| 1475 | CAP binding site | taatgtgagttagctcactcat |
| | (position 646- | |
| | 667) | |
| 1476 | space | taggcaccccaggc |
| | (position 668- | |
| | 681) | |
| 1477 | lac promoter | tttacactttatgcttccggctcgtatgttg |
| | (position 682- | |
| | 712) | |
| 1478 | space | tgtggaattgtgagcggataacaatttcacacaggaaacagct |
| | (position 713- | |
| | 755) | |
| 1479 | lacZa | atgaccatgattacgccaagcttgcatgcctgcaggtcgactctagaggatccccgggtaccgagct |
| | (position 756- | cgaattcactggccgtcgttttacaacgtcgtgactgggaaaaccctggcgttacccaacttaatcg |
| | 1079) | ccttgcagcacatccccctttcgccagctggcgtaatagcgaagaggcccgcaccgatcgcccttcc |
| | | caacagttgcgcagcctgaatggcgaatggcgcctgatgcggtattttctccttacgcatctgtgcg |
| | | gtatttcacaccgcatatggtgcactctcagtacaatctgctctgatgccgcatag |
| 1480 | space | ttaagccagccccgacacccgccaacacccgctgacgcgccctgacgggcttgtctgctcccggca |
| | (position 1080- | tccgcttacagacaagctgtgaccgtctccgggagctgcatgtgtcagaggttttcaccgtcatcac |
| | 1319) | cgaaacgcgcgagacgaaagggcctcgtgatacgcctatttttataggttaatgtcatgataataat |
| | | ggtttcttagacgtcaggtggcacttttcggggaaatgtg |
| 1481 | AmpR promoter | cgcggaacccctatttgtttatttttctaaatacattcaaatatgtatccgctcatgagacaataac |
| | (position 1320- | cctgataaatgcttcaataatattgaaaaaggaagagt |
| | 1424) | |
| 1482 | AmpR | atgagtattcaacatttccgtgtcgcccttattcccttttttgcggcattttgccttcctgtttttg |
| | (position 1425- | ctcacccagaaacgctggtgaaagtaaaagatgctgaagatcagttgggtgcacgagtgggttacat |
| | 2285) | cgaactggatctcaacagcggtaagatccttgagagttttcgccccgaagaacgttttccaatgatg |
| | | agcacttttaaagttctgctatgtggcgcggtattatcccgtattgacgccgggcaagagcaactcg |
| | | gtcgccgcatacactattctcagaatgacttggttgagtactcaccagtcacagaaaagcatcttac |
| | | ggatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacaccac |
| | | gatgcctgtagcaatggcaacaacgttgcgcaaactattaactggcgaactacttactctagcttcc |
| | | cggcaacaattaatagactggatggaggcggataaagttgcaggaccacttctgcgctcggcccttc |
| | | cggctggctggtttattgctgataaatctggagccggtgagcgtgggtctcgcggtatcattgcagc |
| | | actggggccagatggtaagccctcccgtatcgtagttatctacacgacggggagtcaggcaactatg |
| | | gatgaacgaaatagacagatcgctgagataggtgcctcactgattaagcattggtaa |
| 1483 | space | ctgtcagaccaagtttactcatatatactttagattgatttaaaacttcatttttaatttaaaagga |
| | (position 2286- | tctaggtgaagatcctttttgataatctcatgaccaaaatcccttaacgtgagttttcgttccactg |
| | 2455) | agcgtcagaccccgtagaaaagatcaaaggatcttc |
| 1484 | ori | ttgagatcctttttttctgcgcgtaatctgctgcttgcaaacaaaaaaaccaccgctaccagcggtg |
| | (position 2456- | gtttgtttgccggatcaagagctaccaactctttttccgaaggtaactggcttcagcagagcgcaga |
| | 2827) | taccaaatactgttcttctagtgtagccgtagttaggccaccacttcaagaactctgtagcaccgcc |
| | | tacatacctcgctctgctaatcctgttaccagtggctgctgccagtggcgataagtcgtgtcttacc |
| | | gggttggactcaagacgatagttaccggataaggcgcagcggtcgggctgaacggggggttcgtgca |
| | | cacagcccagcttggagcgaacgacctacaccgaact |

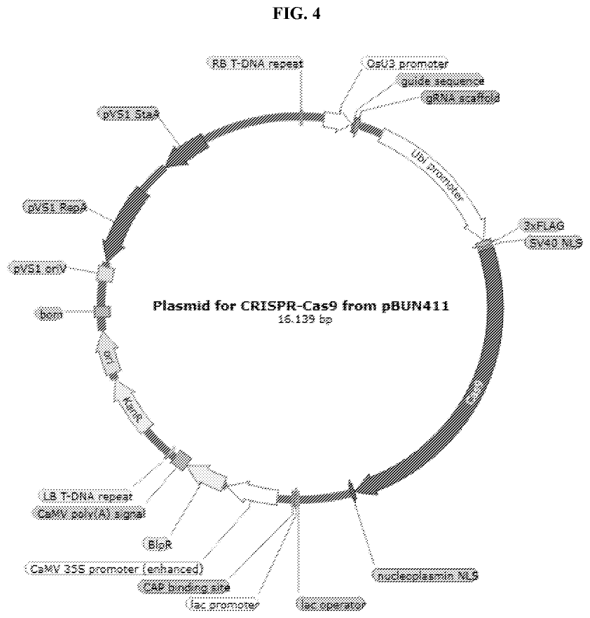

A CRISPR-Cas9 plasmid as described in Table 11 is prepared. The original plasmid is the pBUN411, which is available on https://www.addgene.org/50581/. The guide sequence 29rev from Os03t0718100-01 intron 1 of Table 4 is used. A schematic map of the CRISPR-Cas9 plasmid is shown in FIG. 4.

TABLE 11
CRISPR-Cas 9 Plasmid Sequences (from 5′ to 3′). The first column (SEQ
ID NO) contains the sequence identifier of non-limiting examples of acid nucleic sequences of a
CRISPR-Cas 9 plasmid. The second column (Feature/Position) describes the feature name and the
position of the sequence into the plasmid. The third column (Sequence) contains the acid
nucleic sequence of the referred feature.

| SEQ | | |
|---|---|---|
| ID | | |
| NO. | Position | Sequence |
| 1485 | space | taaacgctcttttctcttag |
| | (position 1-20) | |
| 1486 | RB T-DNA | gtttacccgccaatatatcctgtca |
| | repeat (position | |
| | 21-45) | |
| 1487 | space | aacactgatagtttaaactgaaggcgggaaacgacaatctgatccaagctcaagctgctctagcatt |
| | (position 46- | cgccattcaggctgcgcaactgttgggaagggcgatcggtgcgggcctcttcgctattacgccagct |
| | 330) | ggcgaaagggggatgtgctgcaaggcgattaagttgggtaacgccagggttttcccagtcacgacgt |
| | | tgtaaaacgacggccagtgccaagcttagtaattcatccaggtcaccaagttctaggattttcagaa |
| | | ctgcaacttattttatc |
| 1488 | OsU3 promoter | aaggaatctttaaacatacgaacagatcacttaaagttcttctgaagcaacttaaagttatcaggca |
| | (position 331- | tgcatggatcttggaggaatcagatgtgcagtcagggaccatagcacaagacaggcgtcttctactg |
| | 707) | gtgctaccagcaaatgctggaagccgggaacactgggtacgttggaaaccacgtgatgtgaagaagt |
| | | aagataaactgtaggagaaaagcatttcgtagtgggccatgaagcctttcaggacatgtattgcagt |
| | | atgggccggcccattacgcaattggacgacaacaaagactagtattagtaccacctcggctatccac |
| | | atagatcaaagctgatttaaaagagttgtgcagatgatccgt |
| 1489 | space | ggcg |
| | (position 708- | |
| | 711) | |
| 1490 | guide sequence | aaatgcagcatttcggtaaa |
| | (position 712- | |
| | 731) | |
| 1491 | gRNA scaffold | gttttagagctagaaatagcaagttaaaataaggctagtccgttatcaacttgaaaaagtggcaccg |
| | (position 732- | agtcggtgc |
| | 807) | |
| 1492 | space | ttttttttttcgttttgcattgagttttctccgtcgcatgtttgcagttttattttccgttttgcat |
| | (position 808- | tgaaatttctccgtctcatgtttgcagcgtgttcaaaaagtacgcagctgtatttcacttatttacg |
| | 1110) | gcgccacattttcatgccgtttgtgccaactatcccgagctagtgaatacagcttggcttcacacaa |
| | | cactggtgacccgctgacctgctcgtacctcgtaccgtcgtacggcacagcatttggaattaaaggg |
| | | tgtgatcgatactgcttgctgctaagcttgcatgc |
| 1493 | Ubi promoter | ctgcagtgcagcgtgacccggtcgtgcccctctctagagataatgagcattgcatgtctaagttata |
| | (position 1111- | aaaaattaccacatattttttttgtcacacttgtttgaagtgcagtttatctatctttatacatata |
| | 3102) | tttaaactttactctacgaataatataatctatagtactacaataatatcagtgttttagagaatca |
| | | tataaatgaacagttagacatggtctaaaggacaattgagtattttgacaacaggactctacagttt |
| | | tatctttttagtgtgcatgtgttctcctttttttttgcaaatagcttcacctatataatacttcatc |
| | | cattttattagtacatccatttagggtttagggttaatggtttttatagactaatttttttagtaca |
| | | tctattttattctattttagcctctaaattaagaaaactaaaactctattttagtttttttatttaa |
| | | taatttagatataaaatagaataaaataaagtgactaaaaattaaacaaataccctttaagaaatta |
| | | aaaaaactaaggaaacatttttcttgtttcgagtagataatgccagcctgttaaacgccgtcgacga |
| | | gtctaacggacaccaaccagcgaaccagcagcgtcgcgtcgggccaagcgaagcagacggcacggca |
| | | tctctgtcgctgcctctggacccctctcgagagttccgctccaccgttggacttgctccgctgtcgg |
| | | catccagaaattgcgtggcggagcggcagacgtgagccggcacggcaggcggcctcctcctcctctc |
| | | acggcacggcagctacgggggattcctttcccaccgctccttcgctttcccttcctcgcccgccgta |
| | | ataaatagacaccccctccacaccctctttccccaacctcgtgttgttcggagcgcacacacacaca |
| | | ccatggttagggcccggtagttctacttctgttcatgtttgtgttagatccgtgtttgtgttagatc |
| | | cgaccagatctcccccaaatccacccgtcggcacctccgcttcaaggtacgccgctcgtcctccccc |
| | | cccccccctctctaccttctctagatcggcgttccggttgctgctagcgttcgtacacggatgcgac |
| | | ctgtacgtcagacacgttctgattgctaacttgccagtgtttctctttggggaatcctgggatggct |
| | | ctagccgttccgcagacgggatcgatttcatgattttttttgtttcgttgcatagggtttggtttgc |
| | | ccttttcctttatttcaatatatgccgtgcacttgtttgtcgggtcatcttttcatgcttttttttg |
| | | tcttggttgtgatgatgtggtctggttgggcggtcgttctagatcggagtagaattctgtttcaaac |
| | | tacctggtggatttattaattttggatctgtatgtgtgtgccatacatattcatagttacgaattga |
| | | agatgatggatggaaatatcgatctaggataggtatacatgttgatgcgggttttactgatgcatat |
| | | acagagatgctttttgttcgcttggttgtgatgatgtggtgtggttgggcggtcgttcattcgttct |
| | | agatcggagtagaatactgtttcaaactacctggtgtatttattaattttggaactgtatgtgtgtg |
| | | tcatacatcttcatagttacgagtttaagatggatggaaatatcgatctaggataggtatacatgtt |
| | | gatgtgggttttactgatgcatatacatgatggcatatgcagcatctattcatatgctctaaccttg |
| | | tagtacctatctattataataaacaagtatgttttataattattttgatcttgaatacttggatgat |
| | | ggcatatgcagcagctatatgtggatttttttagccctgccttcatacgctatttatttgcttggta |
| | | ctgtttcttttgtcgatgctcaccctgttgtttggtgttacttctgcag |
| 1494 | space | ccctaggcctactagatg |
| | (position 3103- | |
| | 3120) | |
| 1495 | 3xFLAG | gattacaaggaccacgacggggattacaaggaccacgacattgattacaaggatgatgatgacaag |
| | (position 3121- | |
| | 3186) | |
| 1496 | space | atggct |
| | (position 3187- | |
| | 3792) | |
| 1497 | SV40 NLS | ccgaagaagaagaggaaggtt |
| | (position 3193- | |
| | 3213) | |
| 1498 | space | ggcatccacggggtgccagctgct |
| | (position 2314- | |
| | 3237) | |
| 1499 | Cas9 | gacaagaagtactcgatcggcctcgatattgggactaactctgttggctgggccgtgatcaccgacg |
| | (position 3238- | agtacaaggtgccctcaaagaagttcaaggtcctgggcaacaccgatcggcattccatcaagaagaa |
| | 7338) | tctcattggcgctctcctgttcgacagcggcgagacggctgaggctacgcggctcaagcgcaccgcc |
| | | cgcaggcggtacacgcgcaggaagaatcgcatctgctacctgcaggagattttctccaacgagatgg |
| | | cgaaggttgacgattctttcttccacaggctggaggagtcattcctcgtggaggaggataagaagca |
| | | cgagcggcatccaatcttcggcaacattgtcgacgaggttgcctaccacgagaagtaccctacgatc |
| | | taccatctgcggaagaagctcgtggactccacagataaggcggacctccgcctgatctacctcgctc |
| | | tggcccacatgattaagttcaggggccatttcctgatcgagggggatctcaacccggacaatagcga |
| | | tgttgacaagctgttcatccagctcgtgcagacgtacaaccagctcttcgaggagaaccccattaat |
| | | gcgtcaggcgtcgacgcgaaggctatcctgtccgctaggctctcgaagtctcggcgcctcgagaacc |
| | | tgatcgcccagctgccgggcgagaagaagaacggcctgttcgggaatctcattgcgctcagcctggg |
| | | gctcacgcccaacttcaagtcgaatttcgatctcgctgaggacgccaagctgcagctctccaaggac |
| | | acatacgacgatgacctggataacctcctggcccagatcggcgatcagtacgcggacctgttcctcg |
| | | ctgccaagaatctgtcggacgccatcctcctgtctgatattctcagggtgaacaccgagattacgaa |
| | | ggctccgctctcagcctccatgatcaagcgctacgacgagcaccatcaggatctgaccctcctgaag |
| | | gcgctggtcaggcagcagctccccgagaagtacaaggagatcttcttcgatcagtcgaagaacggct |
| | | acgctgggtacattgacggcggggcctctcaggaggagttctacaagttcatcaagccgattctgga |
| | | gaagatggacggcacggaggagctgctggtgaagctcaatcgcgaggacctcctgaggaagcagcgg |
| | | acattcgataacggcagcatcccacaccagattcatctcggggagctgcacgctatcctgaggaggc |
| | | aggaggacttctaccctttcctcaaggataaccgcgagaagatcgagaagattctgactttcaggat |
| | | cccgtactacgtcggcccactcgctaggggcaactcccgcttcgcttggatgacccgcaagtcagag |
| | | gagacgatcacgccgtggaacttcgaggaggtggtcgacaagggcgctagcgctcagtcgttcatcg |
| | | agaggatgacgaatttcgacaagaacctgccaaatgagaaggtgctccctaagcactcgctcctgta |
| | | cgagtacttcacagtctacaacgagctgactaaggtgaagtatgtgaccgagggcatgaggaagccg |
| | | accggaaggtcacggttaagcagctcaaggaggactacttcaagaagattgagtgcttcgattcggt |
| | | cggctttcctgtctggggagcagaagaaggccatcgtggacctcctgttcaagaccaagatctctgg |
| | | cgttgaggaccgcttcaacgcctccctggggacctaccacgatctcctgaagatcattaaggataag |
| | | gacttcctggacaacgaggagaatgaggatatcctcgaggacattgtgctgacactcactctgttcg |
| | | caggaccgggagatgatcgaggagcgcctgaagacttacgcccatctcttcgatgacaaggtcatga |
| | | agcagctcaagaggaggaggtacacggctgggggaggctgagcaggaagctcatcaacggcattcgg |
| | | gacaagcagtccgggaagacgatcctcgacttcctgaagagcgatggcttcgcgaaccgcaatttca |
| | | tgcagctgattcacgatgacagcctcacattcaaggaggatatccagaaggctcaggtgagcggcca |
| | | tgagaacatcgtcattgagatggcccgggagaatcagaccacgcagaagggccagaagaactcacgc |
| | | gagggggactcgctgcacgagcatatcgcgaacctcgctggctcgccagctatcaagaaggggattc |
| | | tgcagaccgtgaaggttgtggacgagctggtgaaggtcatgggcaggcacaagccgaggatgaagag |
| | | gatcgaggagggcattaaggagctggggtcccagatcctcaaggagcacccggtggagaacacgcag |
| | | ctgcagaatgagaagctctacctgtactacctccagaatggccgcgatatgtatgtggaccaggagc |
| | | tggatattaacaggctcagcgattacgacgtcgatcatatcgttccacagtcattcctgaaggatga |
| | | gctccattgacaacaaggtcctcaccaggtcggacaagaaccggggcaagtctgataatgttccttc |
| | | agaggaggtcgttaagaagatgaagaactactggcgccagctcctgaatgccaagctgatcacgcag |
| | | cggaagttcgataacctcacaaaggctgagagggcgggctctctgagctggacaaggcgggcttcat |
| | | caagaggcagctggtcgagacacggcagatcactaagcacgttgcgcagattctcgactcacggatg |
| | | aacactaagtacgatgagaatgacaagctgatccgcgaggtgaaggtcatcaccctgaagtcaaagc |
| | | tcgtctccgacttcaggaaggatttccagttctacaaggttcgggagatcaacaattaccaccatgc |
| | | ccatgacgcgtacctgaacgcggtggtcggcacagctctgatcaagaagtacccaaagctcgagagc |
| | | gagttcgtgtacggggactacaaggtttacgatgtgaggaagatgatcgccaagtcggagcaggaga |
| | | ttggcaaggctaccgccaagtacttcttctactctaacattatgaatttcttcaagacagagatcac |
| | | tctggccaatggcgagatccggaagcgccccctcatcgagacgaacggcgagacgggggagatcgtg |
| | | tgggacaagggcagggatttcgcgaccgtcaggaaggttctctccatgccacaagtgaatatcgtca |
| | | agaagacagaggtccagactggcgggttctctaaggagtcaattctgcctaagcggaacagcgacaa |
| | | gctcatcgcccgcaagaaggactgggatccgaagaagtacggcgggttcgacagccccactgtggcc |
| | | tactcggtcctggttgtggcgaaggttgagaagggcaagtccaagaagctcaagagcgtgaaggagc |
| | | tgctggggatcacgattatggagcgctccagcttcgagaagaacccgatcgatttcctggaggcgaa |
| | | gggctacaaggaggtgaagaaggacctgatcattaagctccccaagtactcactcttcgagctggag |
| | | gctgttcgtcgagcagcacaagcattacctcgacgagatcattgagcagatttccgagttctccaag |
| | | cgaacggcaggaagcggatgctggcttccgctggcgagctgcagaaggggaacgagctggctctgcc |
| | | gtccaagtatgtgaacttcctctacctggcctcccactacgagaagctcaagggcagccccgaggac |
| | | aacgagcagaagcacgtgatcctggccgacgcgaatctggataaggtcctctccgcgtacaacaagc |
| | | accgcgacaagccaatcagggagcaggctgagaatatcattcatctcttcaccctgacgaacctcgg |
| | | cgcccctgctgctttcaagtacttcgacacaactatcgatcgcaagaggtacacaagcactaaggag |
| | | gtcctggacgcgaccctcatccaccagtcgattaccggcctctacgagacgcgcatcgacctgtctc |
| | | agctcgggggcgac |
| 1500 | nucleoplasmin | aagcggccagcggcgacgaagaaggcggggcaggcgaagaagaagaag |
| | NLS (position | |
| | 7339-7386) | |
| 1501 | space | tgagctcagagctttcgttcgtatcatcggtttcgacaacgttcgtcaagttcaatgcatcagtttc |
| | (position 7387- | attgcgcacacaccagaatcctactgagtttgagtattatggcattgggaaaactgtttttcttgta |
| | 8067) | ccatttgttgtgcttgtaatttactgtgttttttattcggttttcgctatcgaactgtgaaatggaa |
| | | atggatggagaagagttaatgaatgatatggtccttttgttcattctcaaattaatattatttgttt |
| | | tttctcttatttgttgtgtgttgaatttgaaattataagagatatgcaaacattttgttttgagtaa |
| | | caaatgtgtcaaatcgtggcctctaatgaccgaagttaatatgaggagtaaaacacttgtagttgta |
| | | cccattatgcttattactaggcaacaaatatattttcagacctagaaaagctgcaaatgttactgaa |
| | | tacaagtatgtcctcttgtgttttagaatttatgaactttcctttatgtaattttccagaatccttg |
| | | tcagattctaatcattgctttataattatagttatactcatggatttgtagttgagtatgaaaatat |
| | | tttttaatgcattttatgacttgccaattgattgacaacgaattcgtaatcatggtcatagctgttt |
| | | cctgtgtgaaa |
| 1502 | lac operator | ttgttatccgctcacaa |
| | (position 8068- | |
| | 8084) | |
| 1503 | space | ttccaca |
| | (position 8085- | |
| | 8091) | |
| 1504 | lac promoter | caacatacgagccggaagcataaagtgtaaa |
| | (position 8092- | |
| | 8122) | |
| 1505 | space | gcctggggtgccta |
| | (position 8123- | |
| | 8136) | |
| 1506 | CAP binding | atgagtgagctaactcacatta |
| | site (position | |
| | 8137- 8158) | |
| 1507 | space | attgcgttgcgctcactgcccgctttccagtcgggaaacctgtcgtgccagctgcattaatgaatcg |
| | (position 8159- | gccaacgcgcggggagaggcggtttgcgtattggctagagcagcttgccaacatggtggagcacgac |
| | 8348) | actctcgtctactccaagaatatcaaagatacagtctcagaagaccaaagggctat |
| 1508 | CaMV 35S | tgagacttttcaacaaagggtaatatcgggaaacctcctcggattccattgcccagctatctgtcac |
| | promoter | ttcatcaaaaggacagtagaaaaggaaggtggcacctacaaatgccatcattgcgataaaggaaagg |
| | (enhanced) | ctatcgttcaagatgcctctgccgacagtggtcccaaagatggacccccacccacgaggagcatcgt |
| | (position 8349- | tggaaaaagaagacgttccaaccacgtctcaaagcaagtggattgatgtgataacatggtggagcac |
| | 9026) | gacactctcgtctactccaagaatatcaaagatacagtctcagaagaccaaagggctattgagactt |
| | | ttcaacaaagggtaatatcgggaaacctcctcggattccattgcccagctatctgtcacttcatcaa |
| | | aaggacagtagaaaaggaaggtggcacctacaaatgccatcattgcgataaaggaaaggctatcgtt |
| | | caagatgcctctgccgacagtggtcccaaagatggacccccacccacgaggagcatcgtggaaaaag |
| | | aagacgttccaaccacgtcttcaaagcaagtggattgatgtgatatctccactgacgtaagggatga |
| | | cgcacaatcccactatccttcgcaagaccttcctctatataaggaagttcatttcatttggagagga |
| | | cacgctga |
| 1509 | space | aatcaccagtctctctctacaaatctatctctctcgagtctacc |
| | (position 9027- | |
| | 9070) | |
| 1510 | BlpR | atgagcccagaacgacgcccggccgacatccgccgtgccaccgaggcggacatgccggcggtctgca |
| | (9071-9622) | ccatcgtcaaccactacatcgagacaagcacggtcaacttccgtaccgagccgcaggaaccgcagga |
| | | gtggacggacgacctcgtccgtctgcgggagcgctatccctggctcgtcgccgaggtggacggcgag |
| | | gtcgccggcatcgcctacgcgggcccctggaaggcacgcaacgcctacgactggacggccgagtcga |
| | | ccgtgtacgtctccccccgccaccagcggacgggactgggctccacgctctacacccacctgctgaa |
| | | gtccctggaggcacagggcttcaagagcgtggtcgctgtcatcgggctgcccaacgacccgagcgtg |
| | | cgcatgcacgaggcgctcggatatgccccccgcggcatgctgcgggcggccggcttcaagcacggga |
| | | actggcatgacgtgggtttctggcagctggacttcagcctgccggtaccgccccgtccggtcctgcc |
| | | cgtcaccgagatttga |
| 1511 | space | ctcgag |
| | (position 9623- | |
| | 9628) | |
| 1512 | CaMV | tttctccataataatgtgtgagtagttcccagataagggaattagggttcctatagggtttcgctca |
| | poly(A)signal | tgtgttgagcatataagaaacccttagtatgtatttgtatttgtaaaatacttctatcaataaaatt |
| | (position 9629- | tctaattcctaaaaccaaaatccagtactaaaatccagatc |
| | 9803 | |
| 1513 | space | ccccgaattaattcggcgttaattcagtacattaaaaacgtccgcaatgtgttattaagttgtctaa |
| | (position 9804- | gcgtcaattt |
| | 9880) | |
| 1514 | LB T-DNA | gtttacaccacaatatatcctgcca |
| | repeat (position | |
| | 9881-9905) | |
| 1515 | space | ccagccagccaacagctccccgaccggcagctcggcacaaaatcaccactcgatacaggcagcccat |
| | (position 9906- | cagtccgggacggcgtcagcgggagagccgttgtaaggcggcagactttgctcatgttaccgatgct |
| | 10329) | attcggaagaacggcaactaagctgccgggtttgaaacacggatgatctcgcggagggtagcatgtt |
| | | gattgtaacgatgacagagcgttgctgcctgtgatcaccgcggtttcaaaatcggctccgtcgatac |
| | | tatgttatacgccaactttgaaaacaactttgaaaaagctgttttctggtatttaaggttttagaat |
| | | gcaaggaacagtgaattggagttcgtcttgttataattagcttcttggggtatctttaaatactgta |
| | | gaaaagaggaaggaaataataa |
| 1516 | KanR | atggctaaaatgagaatatcaccggaattgaaaaaactgatcgaaaaataccgctgcgtaaaagata |
| | (position 10330- | cggaaggaatgtctcctgctaaggtatataagctggtgggagaaaatgaaaacctatatttaaaaat |
| | 11124) | gacggacagccggtataaagggaccacctatgatgtggaacgggaaaaggacatgatgctatggctg |
| | | gaaggaaagctgcctgttccaaaggtcctgcactttgaacggcatgatggctggagcaatctgctca |
| | | tgagtgaggccgatggcgtcctttgctcggaagagtatgaagatgaacaaagccctgaaaagattat |
| | | cgagctgtatgcggagtgcatcaggctctttcactccatcgacatatcggattgtccctatacgaat |
| | | agcttagacagccgcttagccgaattggattacttactgaataacgatctggccgatgtggattgcg |
| | | aaaactgggaagaagacactccatttaaagatccgcgcgagctgtatgattttttaaagacggaaaa |
| | | gcccgaagaggaacttgtcttttcccacggcgacctgggagacagcaacatctttgtgaaagatggc |
| | | aaagtaagtggctttattgatcttgggagaagcggcagggcggacaagtggtatgacattgccttct |
| | | gcgtccggtcgatcagggaggatatcggggaagaacagtatgtcgagctattttttgacttactggg |
| | | gatcaagcctgattgggagaaaataaaatattatattttactggatgaattgttttag |
| 1517 | space | tacctagaatgcatgaccaaaatcccttaacgtgagttttcgttccactgagcgtcagaccccgtag |
| | (position 11125- | aaaagatcaaaggatcttc |
| | 11210 | |
| 1518 | ori | ttgagatcctttttttctgcgcgtaatctgctgcttgcaaacaaaaaaaccaccgctaccagcggtg |
| | (position 11211- | gtttgtttgccggatcaagagctaccaactctttttccgaaggtaactggcttcagcagagcgcaga |
| | 11799) | taccaaatactgtccttctagtgtagccgtagttaggccaccacttcaagaactctgtagcaccgcc |
| | | tacatacctcgctctgctaatcctgttaccagtggctgctgccagtggcgataagtcgtgtcttacc |
| | | gggttggactcaagacgatagttaccggataaggcgcagcggtcgggctgaacggggggttcgtgca |
| | | cacagcccagcttggagcgaacgacctacaccgaactgagatacctacagcgtgagctatgagaaag |
| | | cgccacgcttcccgaagggagaaaggcggacaggtatccggtaagcggcagggtcggaacaggagag |
| | | cgcacgagggagcttccagggggaaacgcctggtatctttatagtcctgtcgggtttcgccacctct |
| | | gacttgagcgtcgatttttgtgatgctcgtcaggggggcggagcctatggaaa |
| 1519 | space | aacgccagcaacgcggcctttttacggttcctggccttttgctggccttttgctcacatgttctttc |
| | (position 11800- | ctgcgttatcccctgattctgtggataaccgtattaccgcctttgagtgagctgataccgctcgccg |
| | 11984) | cagccgaacgaccgagcgcagcgagtcagtgagcgaggaagcggaagagcg |
| 1520 | bom | cctgatgcggtattttctccttacgcatctgtgcggtatttcacaccgcatatggtgcactctcagt |
| | (position 11985- | acaatctgctctgatgccgcatagttaagccagtatacactccgctatcgctacgtgactgggtcat |
| | 12125) | ggctgcg |
| 1521 | space | ccccgacacccgccaacacccgctgacgcgccctgacgggcttgtctgctcccggcatccgcttaca |
| | (position 12126- | gacaagctgtgaccgtctccgggagctgcatgtgtcagaggttttcaccgtcatcaccgaaacgcgc |
| | 12468) | gaggcagggtgccttgatgtgggcgccggcggtcgagtggcgacggcgcggcttgtccgcgccctgg |
| | | tagattgcctggccgtaggccagccatttttgagcggccagcggccgcgataggccgacgcgaagcg |
| | | gcggggcgtagggagcgcagcgaccgaagggtaggcgctttttgcagctcttcggctgtgcgctggc |
| | | cagacagt |
| 1522 | pVS1 oriV | tatgcacaggccagggggttttaagagttttaataagttttaaagagttttaggcggaaaaatcgcc |
| | (position 12469- | ttttttctcttttatatcagtcacttacatgtgtgaccggttcccaatgtacggctttgggttccca |
| | 12663) | atgtacgggttccggttcccaatgtacggctttgggttcccaatgtacgtgctatccaca |
| 1523 | space | ggaaacagaccttttcgacctttttcccctgctagggcaatttgccctagcatctgctccgtaca |
| | (position 12664- | |
| | 12728) | |
| 1524 | pVS1 RepA | ttaggaaccggcggatgcttcgccctcgatcaggttgcggtagcgcatgactaggatcgggccagcc |
| | (position 12729- | tgccccgcctcctccttcaaatcgtactccggcaggtcatttgacccgatcagcttgcgcacggtga |
| | 13802) | aacagaacttcttgaactctccggcgctgccactgcgttcgtagatcgtcttgaacaaccatctggc |
| | | gttctgccttgcctgcggcgcggcgtgccagcggtagagaaaacggccgatgccgggatcgatcaaa |
| | | aagtaatcggggtgaaccgtcagcacgtccgggttcttgccttctgtgatctcgcggtacatccaat |
| | | cagctagctcgatctcgatgtactccggccgcccggtttcgctctttacgatcttgtagcggctaat |
| | | caaggcttcaccctcggataccgtcaccaggcggccgttcttggccttcttcgtacgctgcatggca |
| | | acgtgcgtggtgtttaaccgaatgcaggtttctaccaggtcgtctttctgctttccgccatcggctc |
| | | cgccggcagaacttgagtacgtccgcaacgtgtggacggaacacgcggccgggcttgtctcccttcc |
| | | cttcccggtatcggttatggattcggttagatgggaaaccgccatcagtaccaggtcgtaatcccac |
| | | acactggccatgccggccggccctgcggaaacctctacgtgcccgtctggaagctcgtagcggatca |
| | | cctcgccagctcgtcggtcacgcttcgacagacggaaaacggccacgtccatgatgctgcgactatc |
| | | gcgggtgcccacgtcatagagcatcggaacgaaaaaatctggttgctcgtcgcccttgggcggcttc |
| | | ctaatcgacggcgcaccggctgccggcggttgccgggattctttgcggattcgatcagcggccgctt |
| | | gccacgattcaccggggcgtgcttctgcctcgatgcgttgccgctgggcggcctgcgcggccttcaa |
| | | cttctccaccaggtcatcacccagcgccgcgccgatttgtaccgggccggatggtttgcgaccgctc |
| | | ac |
| 1525 | space | gccgattcctcgggcttgggggttccagtgccattgcagggccggcagacaacccagccgcttacgc |
| | (position 13803- | ctggccaaccgcccgttcctccacacatggggcattccacggcgtcggtgcctggttgttcttgatt |
| | 14230) | ttccatgccgcctcctttagccgctaaaattcatctactcatttattcatttgctcatttactctgg |
| | | tagctgcgcgatgtattcagatagcagctcggtaatggtcttgccttggcgtaccgcgtacatcttc |
| | | agcttggtgtgatcctccgccggcaactgaaagttgacccgcttcatggctggcgtgtctgccaggc |
| | | tggccaacgttgcagccttgctgctgcgtgcgctcggacggccggcacttagcgtgtttgtgctttt |
| | | gctcattttctctttacctcattaac |
| 1526 | pVS1 StaA | tcaaatgagttttgatttaatttcagcggccagcgcctggacctcgcgggcagcgtcgccctcgggt |
| | (position 14231- | tctgattcaagaacggttgtgccggcggcggcagtgcctgggtagctcacgcgctgcgtgatacggg |
| | 14860) | actcaagaatgggcagctcgtacccggccagcgcctcggcaacctcaccgccgatgcgcgtgccttt |
| | | gatcgcccgcgacacgacaaaggccgcttgtagccttccatccgtgacctcaatgcgctgcttaacc |
| | | agctccaccaggtcggcggtggcccatatgtcgtaagggcttggctgcaccggaatcagcacgaagt |
| | | cggctgccttgatcgcggacacagccaagtccgccgcctggggcgctccgtcgatcactacgaagtc |
| | | gcgccggccgatggccttcacgtcgcggtcaatcgtcgggcggtcgatgccgacaacggttagcggt |
| | | tgatcttcccgcacggccgcccaatcgcgggcactgccctggggatcggaatcgactaacagaacat |
| | | cggccccggcgagttgcagggcgcgggctagatgggttgcgatggtcgtcttgcctgacccgccttt |
| | | ctggttaagtacagcgataaccttcat |
| 1527 | space | gcgttccccttgcgtatttgtttatttactcatcgcatcatatacgcagcgaccgcatgacgcaagc |
| | (position 14861- | tgttttactcaaatacacatcacctttttagacggcggcgctcggtttcttcagcggccaagctggc |
| | 16139) | cggccaggccgccagcttggcatcagacaaaccggccaggatttcatgcagccgcacggttgagacg |
| | | tgcgcgggcggctcgaacacgtacccggccgcgatcatctccgcctcgatctcttcggtaatgaaaa |
| | | acggttcgtcctggccgtcctggtgcggtttcatgcttgttcctcttggcgttcattctcggcggcc |
| | | gccagggcgtcggcctcggtcaatgcgtcctcacggaaggcaccgcgccgcctggcctcggtgggcg |
| | | gtcacttcctcgctgcgctcaagtgcgcggtacagggtcgagcgatgcacgccaagcatgcagccgc |
| | | ctctttcacggtgcggccttcctggtcgatcagctcgcgggcgtgcgcgatctgtgccggggtgagg |
| | | gtagggcgggggccaaacttcacgcctcgggccttggcggcctcgcgcccgctccgggtgcggtcga |
| | | tgattagggaacgctcgaactcggcaatgccggcgaacacggtcaacaccatgcggccggccggcgt |
| | | ggtggtgtcggcccacggctctgccaggctacgcaggcccgcgccggcctcctggatgcgctcggca |
| | | atgtccagtaggtcgcgggtgctgcgggccaggcggtctagcctggtcactgtcacaacgtcgccag |
| | | ggcgtaggtggtcaagcatcctggccagctccgggcggtcgcgcctggtgccggtgatcttctcgga |
| | | aaacagcttggtgcagccggccgcgtgcagttcggcccgttggttggtcaagtcctggtcgtcggtg |
| | | ctgacgcgggcatagcccagcaggccagcggcggcgctcttgttcatggcgtaatgtctccggttct |
| | | agtcgcaagtattctactttatgcgactaaaacacgcgacaagaaaacgccaggaaaagggcagggc |
| | | ggcagcctgtcgcgtaacttaggacttgtgcgacatgtcgttttcagaagacggctgcactgaacgt |
| | | cagaagccgactgcactatagcagcggaggggttggatcaaagtactttgatcccgaggggaaccct |
| | | gtggttggcatgcacatacaaatggacgaacggataaaccttttcacgcccttttaaatatccgtta |
| | | ttctaa |

### Example 2: Methods of Preparing Genetically Edited Cells

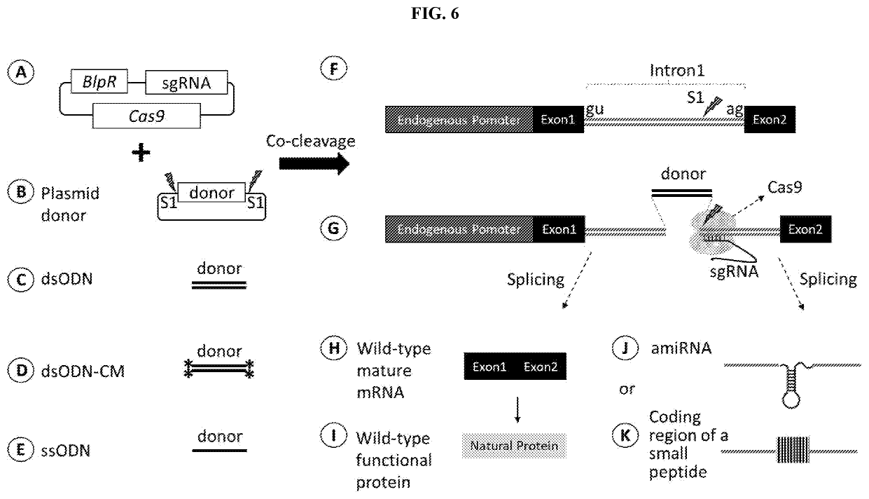

This example further illustrates a non-limiting example of methods of preparing a genetically edited cell as described in FIG. 6A, which depicts a scheme of the plasmid comprising a sequence encoding DNA nuclease (CRISPR associated nuclease-Cas9) and one single guide RNA (sgRNA) which direct the nuclease activity to specific sites of the DNA. The donor DNA comprising the endogenous or exogenous acid nucleic to be inserted into the intron can be delivered by B) a plasmid donor containing two specific sites (S1) of cleavage by Cas9. The two sites S1 are the same present in the intron, thus the co-cleavage occurs in the plasmid donor to lead the donor DNA fragment. The details of both plasmid in A) and B) are described in Example 1.

In another approach, the donor DNA is delivered as C) a blunt linear double-stranded oligodeoxynucleotide (dsODN), or D) a chemically modified dsODN (dsODN-CM) which is flanked by two additional nucleotides with phosphorothioate linkages at the 5′- and 3′-ends of both DNA strands and contain a phosphorylation at the 5′ end of both strand of the exogenous nucleic acid. In another approach, the donor DNA is delivered as a E) a blunt single-stranded oligodeoxynucleotide (ssODN).

Further, F) illustrates schematics of targeted integration of donor DNA containing exogenous nucleic acid into an intron of a gene. The genomic region shows the endogenous promoter (grey box), Exon 1, and Exon 2 (black box) separated by the intron 1 (double lines in grey representing double-strand DNA). The specific site for sgRNA-Cas9 recognition is shown as S1. The 5′splice site GU and 3′splice site AG are shown bearing the intron 1 region. G) The CRISPR-Cas9 system delivered by plasmid as described in Example 1 recognizes the specific site S1 and cleave the double strand of DNA into the intronic region. The donor DNA is inserted into the intron via non-homologous end-joining by the natural DNA repair system present in the cell. After splicing, the natural function of the H) gene and I) protein is preserved.

The other product of splicing is the intronic region containing the endogenous or exogenous nucleic acid, which can be the amiRNA or the coding region of a small peptide. J) The precursor of amiRNA is processed to a mature miRNA and delivered to target the desired trait. K) The intron that comprises a coding region of a small peptide is a template for ribosome machinery binding and is translated into a small peptide with regulatory functions.

### Example 3: Endogenous or Exogenous Nucleic Acids Encoding miRNAs

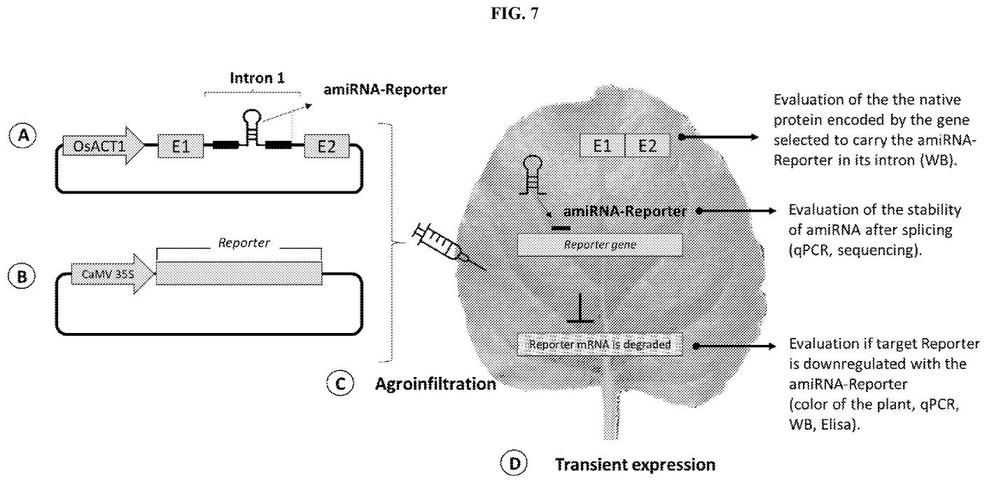

In FIG. 7, A) depicts a scheme of the construct comprising the components to express transiently the cassette containing the amiRNA specific for a reporter gene (amiRNA-Reporter). The cassette comprises the first exon (E1), first intron containing the amiRNA-Reporter, and the second exon (E2) of a gene highly and constitutively expressed selected from Table 1 and Table 2. In this example, ACTIN 1 (SEQ ID NO: 1) from rice of Table 1 and Table 2 is used. The amiRNA-Reporter is inserted at the position described in Table 3 and Table 4 (within SEQ ID NO: 1291). B) depicts a scheme of the plasmid comprising the cassette of the reporter gene overexpression. The reporter gene is driven by a strong promoter commonly used for dicotyledons transient overexpression. The reporter gene is targeted by the amiRNA-Reporter in a specific region. Further, C) both first and second plasmids are used to transform Nicotiana benthamiana leaf via Agroinfiltration. D) the transient co-expression of both the gene highly expressed selected to receive the insertion of the amiRNA-Reporter, the further processed amiRNA-Reporter, and the Reporter gene, were followed and quantified to evaluate: 1) the native protein encoded by the gene selected to receive the insertion of the amiRNA-Reporter in its intron; 2) the presence/stability of amiRNA-Reporter after splicing event; 3) the silencing of the reporter gene target by the amiRNA-Reporter. Techniques for evaluations include Real-time RT-qPCR (qPCR), nucleic acid sequencing, western blotting (WB), ELISA, and phenotype of the leaf (e.g. color or fluorescence).

### Example 4: Exemplary Experiment of Nicotiana benthamiana Leaves Agroinfected with an Agrobacterium Strain Harboring Plasmids

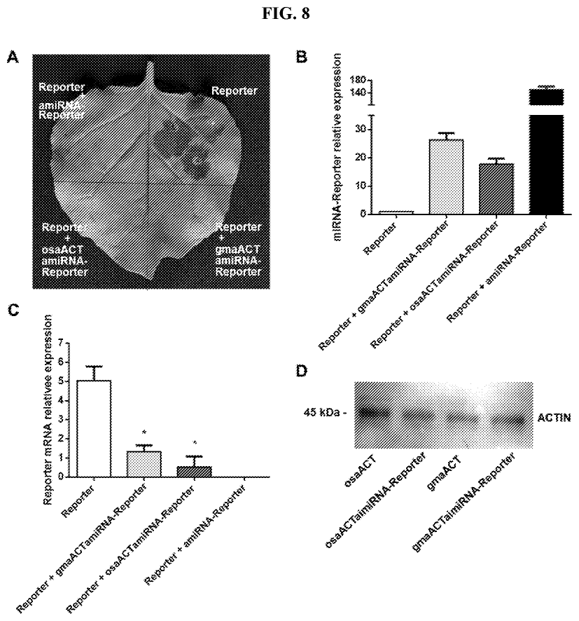

This example further illustrates a non-limiting example of methods of preparing a genetically edited cell as schematically described in FIG. 8. A) The top right leaf quadrant shows the Agroinfection with a control reporter construct. The expression of the reporter gene was visually observed. The top left leaf quadrant shows the co-Agroinfection with both the control reporter construct and a construct comprising an amiRNA (SEQ ID NO: 1532) designed to silence the reporter gene (positive control). The expression of the reporter gene was, visually, completely abolished. The bottom left leaf quadrant shows the co-Agroinfection with both the control reporter construct and a construct comprising an amiRNA (SEQ ID NO:1532) designed to silence the reporter gene inserted into the intron 2 of the rice ACTIN gene (SEQ ID NO: 278). The expression of the reporter gene was, visually, completely abolished. The bottom right leaf quadrant shows the co-Agroinfection with the control reporter construct and a construct comprising an amiRNA (SEQ ID NO: 1532) designed to silence the reporter gene inserted into the intron 2 of the soybean ACTIN gene (SEQ ID NO: 533). The expression of the reporter gene was, visually, completely abolished. B) The amiRNA designed to silence the reporter gene accumulated in the bottom left and bottom right leaf quadrants indicating that the amiRNA inserted into the intron 2 of the actin genes from rice (SEQ ID NO: 1) and soybean (SEQ ID NO: 21) was correctly processed, as determined by qPCR. C) The mRNA transcribed from the reporter gene were targeted and degraded by the amiRNA inserted into the intron 2 of the actin genes from rice and soybean, as determined by qPCR. D) After transcription, splicing, amiRNA processing a mature ACTIN mRNA was produced. After translation, the correct, native ACTIN protein encoded by the rice and the soybean actin genes (SEQ ID NO: 1 and SEQ ID NO: 21, respectively) was produced, as shown by SDS-PAGE.

### Example 5: Endogenous or Exogenous Nucleic Acids Encoding Small Peptide

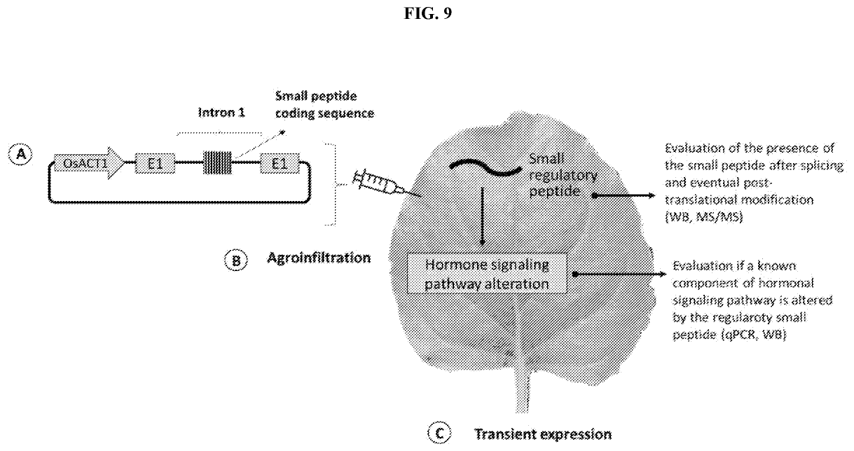

FIG. 9A) depicts a scheme of the plasmid comprising the elements to express transiently the cassette containing small peptide coding sequence. The cassette comprises the first exon (E1), first intron containing the small peptide coding sequence, and the second exon (E2) of a gene highly and constitutively expressed selected from Table 1 and Table 2. ACTIN 1 from rice of Table 1 and Table 2 is used. The small peptide coding sequence is inserted at the position described in Table 3 and Table 4. B) the plasmid is used to transform Nicotiana benthamiana leaf, via Agroinfiltration. Further, C) the transient expression of the small peptide and its effect in the cell is followed and quantified to evaluate: 1) the presence/stability of the small peptide after splicing event and eventual post-translational modification; 2) the effect of the overexpression of the small peptide in its related pathway (e.g. quantification of some target downstream of the hormone signaling pathway). Techniques for evaluations include Real-time RT-qPCR (qPCR), mass spectrometry (MS/MS), western blotting (WB), ELISA, and phenotype of the leaf (e.g. color or fluorescence).

### Example 6: Genetically Edited Plants with Desirable Traits

In FIG. 10, A) depicts a scheme of the genomic region of an endogenous gene from the model plant Arabidopsis. The exons are represented by grey boxes, the intronic regions are along the line bearing each grey box. The amiRNA specific for the target exemplified by a reporter gene (amiRNA-Reporter) is inserted in the intron 6, an intronic region between exon 6 and exon 7. The insertion is by CRISPR-Cas9 and non-homologous end join system (NHEJ), at the specific position exemplified in Table 3 and Table 4. Primers forward (P1) and reverse (P2) are designed to amplify the region of insertion followed by sequencing, to verify the insertion. B) The natural function of the gene comprising the insertion of the amiRNA-Reporter is evaluated by quantifying the mature mRNA and its protein. The presence of mature amiRNA-Reporter is also quantified. C) A previously obtained transgenic Arabidopsis thaliana overexpressing the Reporter (CaMV 35S: Reporter) is the host of the gene editing described in A) and B).

As shown in FIG. 10, the transgenic CaMV 35S: Reporter plant presents red color and when engineered with amiRNA-Reporter is expected to rescue the natural green color. The transgenic CaMV 35S: Reporter plant not engineered with amiRNA-Reporter do not contain the desirable trait of rescuing natural green color). Techniques for evaluations include Real-time RT-qPCR (qPCR), sequencing, western blotting (WB), Elisa, and phenotype of the plant (e.g. color). Some examples of reporter genes are GFP, RFP, anthocyanin, β-glucoronidase (GUS).

This example illustrates that the engineered plant exhibits a desirable trait as compared to a non-engineered plant.

The preceding merely illustrates the principles of this disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of this disclosure and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

1. A system comprising a first nucleic acid sequence comprising a nucleic acid encoding a ribonucleic acid or a peptide, a second nucleic acid sequence comprising a sequence encoding a DNA nuclease, and a third nucleic acid sequence comprising a sequence encoding a guide RNA, wherein the guide RNA is complementary to a non-coding region of the genome of a cell.

2. The system of claim 1, wherein the nucleic acid encodes the ribonucleic acid, and the ribonucleic acid specifically binds to (i) a target nucleic acid of Table 6, (ii) a target nucleic acid present in a pest of Table 6, (iii) a target nucleic acid of an organism of Table 6, (iv) a target nucleic exogenous or endogenous to the cell, (v) a target nucleic acid responsible for water acquisition, nutrient acquisition, disease control, or pest control, or any combination of two or more thereof, in the cell, (vi) a target nucleic acid comprises a regulatory element involved in: plant growth and development, yield, biotic stress, abiotic stress, or herbicide resistance, or any combination of two or more thereof, (vii) a target nucleic acid of an insect, bacteria, fungi, or worm, or a combination of two or more thereof, that is harmful to the cell, (viii) a target nucleic acid of an organism that causes a disease to the cell, or (ix) a combination of two or more of (i) to (viii).

3. (canceled)

4. The system of claim 1, wherein the nucleic acid encodes the peptide, and the peptide is (i) a peptide selected from Table 7, (ii) a peptide encoded by an mRNA sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8, (iii) a peptide that affects hormonal regulation, protection against a pathogen, protection against an insect, nitrogen fixation, nutrient acquisition, immunity induction, biotic stress, or abiotic stress, or a combination of two or more thereof, in the cell, or (iv) a combination of two or more of (i) to (iii).

5. (canceled)

6. The system of claim 1, wherein the non-coding region is positioned within, or adjacent to, a gene of the cell selected from actin, ubiquitin, ribosomal gene, gene encoding a heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, and a gene selected from Table 1.

7.-16. (canceled)

17. A method of inserting the nucleic acid encoding the ribonucleic acid or the peptide into the non-coding region of the cell, the method comprising introducing the system of claim 1 into the cell.

18.-19. (canceled)

20. A cell comprising a recombinant nucleic acid comprising a coding region and a non-coding region, wherein the non-coding region comprises a nucleic acid exogenous to the non-coding region, and wherein the coding region is the coding region of a gene, and the gene (i) is actin, ubiquitin, ribosomal gene, gene encoding a heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, or a gene selected from Table 1; (ii) accounts for about 1% to about 20% of gene expression in the cell; (iii) is transcribed from a constitutive promoter, optionally wherein the promoter is specific or a plant organ or tissue, further optionally wherein the organ or tissue comprises a root, stem, fruit, seed, leaf, ground tissue, vascular tissue, or dermal tissue, or a combination of two or more thereof; or (iv) a combination of two or more of (i) to (iii).

21. The cell of claim 20, wherein the non-coding region comprises (i) an intron positioned between a first exon region of the coding region and a second exon region of the coding region, (ii) a 5′ non-coding region positioned adjacent to the coding region, or (iii) a 3′ non-coding region positioned adjacent to the coding region.

22. The cell of claim 20, wherein the gene encodes mRNA endogenous to the cell, and after transcription of the gene and mRNA splicing, the mRNA is translated into a protein endogenous to the cell.

23. (canceled)

24. The cell of claim 20, wherein the nucleic acid exogenous to the non-coding region encodes a ribonucleic acid or a peptide, and (a) wherein the nucleic acid encodes the ribonucleic acid, and the ribonucleic acid specifically binds to (i) a target nucleic acid of Table 6, (ii) a target nucleic acid present in a pest of Table 6, (iii) a target nucleic acid of an organism of Table 6, (iv) a target nucleic exogenous or endogenous to the cell, (v) a target nucleic acid responsible for water acquisition, nutrient acquisition, disease control, or pest control, or any combination of two or more thereof, in the cell, (vi) a target nucleic acid comprises a regulatory element involved in: plant growth and development, yield, biotic stress, abiotic stress, or herbicide resistance, or any combination of two or more thereof, (vii) a target nucleic acid of an insect, bacteria, fungi, or worm, or a combination of two or more thereof, that is harmful to the cell, (viii) a target nucleic acid of an organism that causes a disease to the cell, or (ix) a combination of two or more of (i) to (viii); or (b) wherein the nucleic acid encodes the peptide, and the peptide is (i) a peptide selected from Table 7, (ii) a peptide encoded by an mRNA sequence at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8, (iii) a peptide that affects hormonal regulation, protection against a pathogen, protection against an insect, nitrogen fixation, nutrient acquisition, immunity induction, biotic stress, or abiotic stress, or a combination of two or more thereof, in the cell, or (iv) a combination of two or more of (i) to (iii).

25.-31. (canceled)

32. The cell of claim 20, wherein the nucleic acid exogenous to the non-coding region is about 10 to about 700 bases in length, or about less than 200 bases in length.

33. A cell comprising a recombinant nucleic acid comprising a coding region and a non-coding region, wherein the non-coding region comprises a nucleic acid exogenous to the non-coding region, and wherein the nucleic acid exogenous to the non-coding region encodes a ribonucleic acid that specifically binds to (i) a target nucleic acid of Table 6, (ii) a target nucleic acid present in pest of Table 6, (iii) a target nucleic acid of an organism of Table 6, (iv) a target nucleic exogenous or endogenous to the cell, (v) a target nucleic acid responsible for water acquisition, nutrient acquisition, disease control, or pest control, or any combination of two or more thereof, in the cell, (v) a target nucleic acid comprises a regulatory element involved in: plant growth and development, yield, biotic stress, abiotic stress, or herbicide resistance, or any combination of two or more thereof, (vi) a target nucleic acid of an insect, bacteria, fungi, or worm (e.g., larva of the insect, and nematode), or a combination of two or more thereof, that is harmful to the cell, (vii) a target nucleic acid of an organism that causes a disease to the cell, or (viii) a combination of two or more of (i) to (vii).

34.-36. (canceled)

37. The cell of claim 33, wherein the non-coding region is positioned within, or adjacent to, a gene of the cell, wherein the gene is actin, ubiquitin, ribosomal gene, gene encoding a heat shock protein, rubisco, tubulin, TMM, FAMA, rbc-S, CAB2, Rac, GLP, PDX1, BiGSSP, Lhca3, SMB, GATA23, ARF, SIREO, Prx, TIP2, ET304, TobRB7, or a gene selected from Table 1.

38.-39. (canceled)

40. The cell of claim 33, wherein the recombinant nucleic acid is positioned within the genome of the cell.

41. The cell of claim 20, wherein the cell is a plant cell, and optionally the plant is a plant of Table 9, and further optionally the plant cell is a ground tissue cell, a vascular tissue cell, or a dermal tissue cell.

42.-43. (canceled)

44. The plant of claim 41, wherein the plant is resistant or more resistant to a pest, disease, or chemical, or a combination of two or more thereof, as compared to a plant that does comprise the cell with the recombinant nucleic acid.

45. The plant of claim 41, wherein the plant has an improved nutritional quality, increased crop yield, more efficient nutrient acquisition, or more efficient photosynthetic efficiency, or a combination of two or more thereof, as compared to a plant that does not comprise the cell with the recombinant nucleic acid.

46. A seed of the plant of claim 41.

47. A method of reducing or eliminating expression of a target gene in the cell of claim 20, the method comprising introducing into the non-coding region of the cell the nucleic acid exogenous to the non-coding region, wherein nucleic acid exogenous to the non-coding region encodes for a sequence that binds to mRNA of the target gene, thereby reducing or eliminating expression of the target gene.

48. A method of regulating a target gene or peptide in the cell of any claim 20, the method comprising introducing into the non-coding region of the cell the nucleic acid exogenous to the non-coding region, wherein the nucleic acid exogenous to the non-coding region encodes for an amino acid sequence that is capable of regulating the target gene or peptide in the cell, thereby regulating the target gene or peptide in the cell.

49. A method of introducing, increasing, or reducing a trait in the plant of claim 43, the method comprising introducing into the non-coding region of the cell of the plant the nucleic acid exogenous to the non-coding region, wherein: the nucleic acid exogenous to the non-coding region encodes for a sequence that binds to mRNA of a target gene, thereby introducing, increasing, or reducing the trait in the plant, or the nucleic acid exogenous to the non-coding region encodes an amino acid sequence that regulates a target gene or peptide in the cell, thereby introducing, increasing or reducing the trait in the plant.

50. (canceled)
